SEQUENCE LISTING (26 sequences)

SEQ ID NO: 1 - DNA, 5238 bp, Artificial Sequence
  Synthetic
ttctctgtcacagaatgaaaatttttctgtcatctcttcgttattaatgtttgtaattgactgaatatcaacgcttatttgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggtagacctcggccgtcgcggcgcttgccggtggtgctgaccccggatgaagtggttcgcatcctcggttttctggaaggcgagcatcgtttgttcgcccaggactctagctatagttctagtggttggcctacgtacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcagctgctagcaccatggctcgagatcccgggtgatcaagtcttcgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcaaaggcctacgtcgacgagctcactagtcgcggccgctttcgaatctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctacttt

SEQ ID NO: 2 - DNA, 1336 bp, Artificial Sequence
  Synthetic
gttaaacgttttgttttctagcaatggcaaacaagcttttcctcgtctgcgcaactttcgccctctgcttcctcctcaccaacgccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcaccaccatcaccatcactccgcggacgaagttgattctgaggcattcgcctgcaagccctgcttcagcacccacctctcagatattgagaccaacacgaccgccgctgccggtttcatggtccttcaggacatcaactgcttccgacctcacggagtctcagcagctcaagagaagatttccttcggaaagagctcccagtgccgtgaagccgtcggtactccacagtacatcaccatcaccgctaacgttaccgacgaatcatacttgtacaacgctgacctcttgatgctttccgcttgccttttctacgcctcagaaatgagcgagaagggcttcaaagtcatcttcggaaacgtctctggcgttgtttccgcttgcgtcaacttcacagattacgtggcccacgttacccaacacacccagcagcaccatctcgttattgatcacatcagattgctccacttccttacaccaagcaaagacgagctttgaactctctctttctctctctccaagtctccttcactctttcg

SEQ ID NO: 3 - DNA, 1175 bp, Artificial Sequence
  Synthetic
aaaggatattcgatattctatcatctttaacatcaagatggctagaggtaaacaaaattctaatcagattcaaaacaatagtaacgcaaacggcaagcgccgtaagcgtaatcgaaggaatcgcaatccgcagacggttcccaactttaaccccattgtcgcaaagccgacggttgccccacttcaaactaacattagaagtgctaggagtgacgttaatgccatcactgttttaaatggcagtgattttcttacaactgtcaaagtccgaggttctaacaatttaactgattccaagtctagaatcttggttaagcaaccaatttctgcgagttcttttcttggtaccagaatttctggtctatcgcaattttgggagcgttatagatggcacaaggctgcagttagatatgttcctgcagtacccaatactttagcttgccaacttattggttacatcgatacagatccactagatgaccccaacgttatcctcgatgtcgatcagttacttaggcaggctacgtcacaagtgggtgcgcggcagtggaatttctctgatacaacaactattccattgattgtcaggcgtgatgatcaattgtactatactggtcaagataaagagaacgttcgtttctctcaacagggtgtattttacctcttgcaagtgactacactacttaatattagtggtgaagccattacaaatgatttaatttcaggttcactatatttagattgggtctgtggattttccatgccacaaattaatcctacaccagtggaagtttcacagctaacttacaatgcggatactattggcaattgggttccaccaacagaactcaagcaaacttatactcaagatattactggtttgaagccaaattctaaatttattatcgtaccctatatggatagagtaagttctgaagtactgcagaagtgcacaatcacttgtaatgaggttgacgcagttggttcaatctcatatttcgatactagcgctatcaaatgtgatgggtacatatcatttcaggccaatagcattggtgaagcaaccttcaccttagtgaccgattatcagggtgcggttgaccctaaaccctatcagtataggattatcagagctatcgtcggcaataattaggttgtcatatctagcacatgat

SEQ ID NO: 4 - DNA, 3202 bp, Artificial Sequence
  Synthetic
gttaaacgttttgttttctagcaatgaagtgcaataatcttctctacggtgtggaactccccatgtgcttgcaatcgagcatgggcatggtggttgttaccggactcgccatagtgtatttggtaatttacatcctcatttactcatttacacatggaccacgactcagccgcagattagcttcctatgtggtaatcggaccttatgaagctattgctaaaagccgacccgcgatggccctacaaagggccatggttgatttgacacgacgggacgtgcgtactgactggtacccattgaataatttgttgatgaacaaacctcaccgcttatctgaaaatggacacaaaacttctggagcggttcgagatgcagctagaaatttaattactagcgctataacatcactgggtatggataagtatgaaatatcaccaggtggccacactgtcgacgagcaattggcatctcatcgacattatgctgttaatgacttacatcgagcatcagcagatgacgcagttaaagaaaacgctgtcattgtggctattgatacggactactatctacgtgatccatcaatctacttttcaaataataatccatttattctgcatacttttcaaccaataactgttgcaggtaaagatggagatgtgaggttcactattagtgacaaccaagtggactatagggtagatggtggtggtaggtggcaacataaagtttggaattggtgtgattatggtgaattcctatatttaaagaacatcctaggtattcttagcattaattggtggctaagtttctttggtatcagaaaggttatataccaaaagatccaacacgcacgtccctgggtggattgtccgaaccgggccttagtttggggattacctcaattcactaggtatatgatwacttggttgccaattgagatgaacgcacgtgaattgagtagagtcaactatcagtgtgctagcagacctggctggaattgtttagttgaccacactagcaccaaacttgtagccagcataggtcgagagggtaatgactgtcatgttgaacttgctaaggaggacttagatgttgttctaggcttaagtaccatgcagtcagttacttcaagacttttacaaatgggttacaagcaaccacaaactttggctacggtttgtcagttctacaacagtgccgcttatgatatactaagctgtagcatagttgctagaccttcacaaccacctgtccactggccattggcgtctgaaattgatcagcctacaacttcttttcgtaattactccaacaatattgtgacctgtggtaatctttgcccccaacttaaacgttgggaggttttaagtaattctttagaacatcgtgttaccatggtggccaataataaggttccaacacctcgcatagccaggtttgctgaagaatacgtccgcctggtagttcccgaagcgaatgtaggtgtaccttatagtttggaagatgcgcgtaaagaacttgataaacccacgcaagtcaatgcggtaaaccaaatttgggaaacagtcgacatggaagtccgccgattaattgaagcatttgtgaagaatgaaccgaccaataaatctggtcgcataatatcatcctttgcggactcaagattcttgttgaaattttccacatatacgcttgcctttcgagatgaagtattacatgctgaacataatcgacattggttttgtcctggattgacacctaatgagatagcagataaagtctgcgactatgttcgtggtgttgcgacacctgcagaaggtgattttagcaactttgacggaagagtatctgcttggtgtcaagagaacgtgatgaatgcggtttaccatagatggtttaaccgtaagttttccaaggaattgcagaagtatacatcaatgttggttagttgcccagctcgagctaagcgttttggtttccaatatgaaccgggagtgggggttaagagtggtagtccaaccacctgtgaccttaattcagttctaaataacttcactcaatacgcagcagttaggctgactaaaccagacctctcaccacaagaagcctttgaacaaactggcttaagtttcggcgacgattcactatttgacaagcaatatcaactcagatggaattacgtcgtcgaacaacttggtatggaactcaaggttgaaccctttgaccccagtaatggtgtgacttttcttgctcgtgtttttcctgatccttatagtacaaacactagttttcaggatccactaagaacgtggagaaagttgaacatgacttcacgcacgtgtgtacctgtcgagtcagcagctcttgatcgtgtcagtggatatttagtaactgacaagtactctccagtaacaagtgagtactgtcacatgattgagaggtgttatatgaacactgccgaaagtgtaactcgaaggagacaacgcaaggattgtgaccgtgagaaaccatactggttagtgagtggtggcgcctggccccagagggagggggactatgaattgatgatgagggttaccgcagcccgtactggatttgaggagtcaaaacttattaatttgattagccagttccaatcagttcatgatccttgggacatcaaaccgctagactatcaggaaccatcaccttataaagacacactagacatagatggccagccggtagatgcagtggacgaacgtcaatatcaaaatgagcgcaacacagtcaacttacgagctggtgcagcaatttcccagatgcctgtcccaagtgtgccaggcggtgaagactgcaatcgacagtctgccgacatgccaggacccaaaagtagcgaaggatctgagcagctacaaggcttgcctgagcaagatggaagcaaccgccttcaatgccaccgacaacctactttcaaagccaagggtggtagcaacactaaagggagaagccgtaaatcccggaacggaggacgttctatcagcagcaaaacagcaaatccaacaactcacgaggttggtggaggcaatggaaagaccagagttaccattgctaagcgaagcggacctaagcgacctcataacgtggtaagcaatgcctgggaaactctgaatgacaaccgataaactctctctttctctctctccaagtctccttcactctttcg

SEQ ID NO: 5 - DNA, 1175 bp, Artificial Sequence; identical to SEQ ID NO: 3
  Synthetic

SEQ ID NO: 6 - DNA, 1336 bp, Artificial Sequence; identical to SEQ ID NO: 2
  Synthetic

SEQ ID NO: 7 - DNA, 939 bp, Artificial Sequence
  Synthetic
cctctcagatattgagaccaacacgaccgccgctgccggtttcatggtccttcaggacatcaactgcttccgacctcacggagtctcagcagctcaagagaagatttccttcggaaagagctcccagtgccgtgaagccgtcggtactccacagtacatcaccatcaccgctaacgttaccgacgaatcatacttgtacaacgctgacctcttgatgctttccgcttgccttttctacgcctcagaaatgagcgagaagggcttcaaagtcatcttcggaaacgtctctggcgttgtttccgcttgcgtcaacttcacagattacgtggcccacgttacccaacacacccagcagcaccatctcgttattgatcacatcagattgctccacttccttacaccaagcaaagacgagctttgaactctctctttctctctctccaagtctccttcactctttcgttttattctatcattcgaaagagtgaaggagacttggagagagagaaagagagagttcaaagctcgtctttgcttggtgtaaggaagtggagcaatctgatgtgatcaataacgagatggtgctgctgggtgtgttgggtaacgtgggccacgtaatctgtgaagttgacgcaagcggaaacaacgccagagacgtttccgaagatgactttgaagcccttctcgctcatttctgaggcgtagaaaaggcaagcggaaagcatcaagaggtcagcgttgtacaagtatgattcgtcggtaacgttagcggtgatggtgatgtactgtggagtaccgacggcttcacggcactgggagctctttccgaaggaaatcttctcttgagctgctgagactccgtgaggtcggaagcagttgatgtcctgaaggaccatgaaaccggcagcggcggtcgtgttggtctcaatatctgagagg

SEQ ID NO: 8 - DNA, 1239 bp, Artificial Sequence
  Synthetic
atggatctttctttcactctttcggtcgtgtcggccatcctcgccatcactgctgtgattgctgtatttattgtgatttttaggtatcacaacactgtgaccaagaccatcgaaacccacacaggcaatatcgagacaaacatggatgaaaacctccgcattcctgtgactgctgaggttggatcaggctacttcaagatgactgatgtgtcctttgacagcgacaccttgggcaaaatcaagatccgcaatggaaagtctgatgcacagatgaaggaagaagatgcggatcttgtcatcactcccgtggagggccgagcactcgaagtgactgtggggcagaatctcacctttgagggaacattcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgagttttattctatcattctcggtctcagtgccagagtaggtgacgtgcacgtacatgtcgaaaagatttccaccggcggtagctgcaattggtgcgccaaaggtggtaccacacacaaaggtgccaacttcatcctcatcaatagagacgggggtgaaggaggaggtgttggagctaccgacaaaggcctttgatgggttaatctttggcaccatctgcataccagtgatgttgatctttcttgatgtgttgttccacaccttgaatgttccctcaaaggtgagattctgccccacagtcacttcgagtgctcggccctccacgggagtgatgacaagatccgcatcttcttccttcatctgtgcatcagactttccattgcggatcttgattttgcccaaggtgtcgctgtcaaaggacacatcagtcatcttgaagtagcctgatccaacctcagcagtcacaggaatgcggaggttttcatccatgtttgtctcgatattgcctgtgtgggtttcgatggtcttggtcacagtgttgtgatacctaaaaatcacaataaatacagcaatcacagcagtgatggcgaggatggccgacacgaccgaaagagtgaaagaaagatccat

SEQ ID NO: 9 - DNA, 423 bp, Artificial Sequence
  Synthetic
gctggtgggggatgatactagtagatatgaagaagtgatgaagacttttgatactgttgaggcagtcaggaagagtgatctagatgaccgtgtttacatggtgtgcctaaagcagggatctacttttgtcctcaatggaggcatcgaagaattgcgtcttttgactggagattcaacgctggagattcaacccatgattgtgccttttattctatcattggcacaatcatgggttgaatctccagcgttgaatctccagtcaaaagacgcaattcttcgatgcctccattgaggacaaaagtagatccctgctttaggcacaccatgtaaacacggtcatctagatcactcttcctgactgcctcaacagtatcaaaagtcttcatcacttcttcatatctactagtatcatcccccaccagc

SEQ ID NO: 10 - DNA, 741 bp, Artificial Sequence
  Synthetic
atggccaccacgactaacactcttcctttcggcaggaccggagcccaggccgctggcccttcttacaccatggaagatcttgaaggctccatgtctatggctcgcatgggtctctttttgatcgttgctatctcaattggtatcctcgtcctggccgtcatgaatgtatggatgggaccaaagaaggacagcgattctgacactgataaggtcaccgatgatgatgacgacactgccaacgataacgatgatgaggacaaatataagaacaggaccagggatatgatgcttctggctgggtccgctcttctgttcctcgtttccgccgccaccgtttttatgtcttaccccaagaggaggcagttttattctatcattctgcctcctcttggggtaagacataaaaacggtggcggcggaaacgaggaacagaagagcggacccagccagaagcatcatatccctggtcctgttcttatatttgtcctcatcatcgttatcgttggcagtgtcgtcatcatcatcggtgaccttatcagtgtcagaatcgctgtccttctttggtcccatccatacattcatgacggccaggacgaggataccaattgagatagcaacgatcaaaaagagacccatgcgagccatagacatggagccttcaagatcttccatggtgtaagaagggccagcggcctgggctccggtcctgccgaaaggaagagtgttagtcgtggtggccat

SEQ ID NO: 11 - protein, 948 aa, Artificial Sequence
  Synthetic
MKCNNLLYGVELPMCLQSSMGMVVVTGLAIVYLVIYILIYSFTHGPRLSRRLASYVVIGPYEAIAKSRPAMALQRAMVDLTRRDVRTDWPLNNLLMNKPHRLSENGHKTSGAVRDAARNLITSAITSLGMDKYEISPGGHTVDEQLASHRHYAVNDLHRASADDAVKENAVIVAIDTDYYLRDPSIYFSNNNPFILHTFQPITVAGKDGDVRFTISDNQVDYRVDGGGRWQHKVWNWCDYGEFLYLKNILGILSINWWLSFFGIRKVIYQKIQHARPWVDCPNRALVWGLPQFTRYMITWLPIEMNARELSRVNYQCASRPGWNCLVDHTSTKLVASIGREGNDCHVELAKEDLDVVLGLSTMQSVTSRLLQMGYKQPQTLATVCQFYNSAAYDILSCSIVARPSQPPVHWPLASEIDQPTTSFRNYSNNIVTCGNLCPQLKRWEVLSNSLEHRVTMVANNKVPTPRIARFAEEYVRLVVPEANVGPYSLEDARKELDKPTQVNAVNQIWETVDMEVRRLIEAFVKNEPTNKSGRIISSFADSRFLLKFSTYTLAFRDEVLHAEHNRHWFCPGLTPNEIADKVCDYVRGVATPAEGDFSNFDGRVSAWCQENVMNAVYHRWFNRKFSKELQKYTSMLVSCPARAKRFGFQYEPGVGVKSGSPTTCDLNSVLNNFTQYAAVRLTKPDLSPQEAFEQTGLSFGDDSLFDKQYQLRWNYVVEQLGMELKVEPFDPSNGVTFLARVFPDPYSTNTSFQDPLRTWRKLNMTSRTCVPVESAALDRVSGYLVTDKYSPVTSEYCHMIERCYMNTAESVTRRRQRKDCDREKPYWLVSGGAWPQREGDYELMMRVTAARTGFEESKLINLISQFQSVHDPWDIKPLDYQEPSPYKDTLDIDGQPVDAVDERQYQNERNTVNLRAGAAISQMPVPSVPGGEDCNRQSADMPGPKSS

SEQ ID NO: 12 - protein, 372 aa, Artificial Sequence
  Synthetic
MARGKQNSNQIQNNSNANGKRRKRNRRNRNPQTVPNFNPIVAKPTVAPLQTNIRSARADVNAITVLNGSDDFLTTVKVRGSNNLTDSKSRILVKQPISASSFLGTRISGLSQFWERYRWHKAAVRYVPAVPNTLACQLIGYIDTDPLDDPNVILDVDQLLRQATSQVGARQWNFSDTTTIPLIVRRDDQLYYTGQDKENVRFSQQGVFYLLQVTTLLNISGEAITNDLISGSLYLDWVCGFSMPQINPTPVEVSQLTYNADTIGNWVPPTELKQTYTQDITGLKPNSKFIIVPYMDRVSSEVLQKCTITCNEVDAVGSISYFDTSAIKCDGYISFQANSIGEATFTLVTDYQGAVDPKPYQYRIIRAIVGNN

SEQ ID NO: 13 - protein, 423 aa, Artificial Sequence
  Synthetic
MANKLFLVCATFALCFLLTNAVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKHHHHHHSADEVDSEAFACKPCFSTHLSDIETNTTAAAGFMVLQDINCFRPHGVSAAQEKISFGKSSQCREAVGTPQYITITANVTDESYLYNADLLMLSACLFYASEMSEKGFKVIFGNVSGVVSACVNFTDYVAHVTQHTQQHHLVIDHIRLLHFLTPSKDEL

SEQ ID NO: 14 - DNA, 21 bp, Artificial Sequence
  Synthetic
ccgtaagcgtaatcgaaggaa

SEQ ID NO: 15 - DNA, 17 bp, Artificial Sequence
  Synthetic
cggctttgcgacaatgg

SEQ ID NO: 16 - DNA, 21 bp, Artificial Sequence
  Synthetic
cgcaatccgcagacggttccc

SEQ ID NO: 17 - DNA, 20 bp, Artificial Sequence
  Synthetic
gcgttatagatggcacaagg

SEQ ID NO: 18 - DNA, 20 bp, Artificial Sequence
  Synthetic
agctgtgaaacttcaactgg

SEQ ID NO: 19 - DNA, 22 bp, Artificial Sequence
  Synthetic
cggcgtgagtatgattctcaaa

SEQ ID NO: 20 - DNA, 21 bp, Artificial Sequence
  Synthetic
atgagcagacacgcagctttt

SEQ ID NO: 21 - DNA, 27 bp, Artificial Sequence
  Synthetic
aaaagtctacgttcaccacgcgccaaa

SEQ ID NO: 22 - DNA, 11 bp, Artificial Sequence
  Synthetic
aaaaaaaaaaa

SEQ ID NO: 23 - DNA, 11 bp, Artificial Sequence
  Synthetic
ttattctatca

SEQ ID NO: 24 - RNA, 33 nt, Artificial Sequence
  Synthetic
cugagaccgaguuauucuaucacucggucucag

SEQ ID NO: 25 - RNA, 43 nt, Artificial Sequence
  Synthetic
uucucacuuucuuucuagguauaccuagaaagaaaguggagaa

SEQ ID NO: 26 - RNA, 44 nt, Artificial Sequence
  Synthetic
accgugacucuggcucacuauccuuauugagccagagucacggu

(19) United States
(12) Patent Application Publication
(10) Pub. No.: US 2024/0318202 A1
(43) Pub. Date: Sep. 26, 2024

(54) ENGINEERED NODAVIRAL CARGO DELIVERY SYSTEMS

(71) Applicant: ARIZONA BOARD OF REGENTS ON BEHALF OF THE UNIVERSITY OF ARIZONA, Tucson, AZ (US)

(72) Inventors: Arun Dhar, Tucson, AZ (US); Arjoneel Dhar, Tucson, AZ (US); Rod Russel R. Alenton, Tucson, AZ (US)

(21) Appl. No.: 18/574,777

(22) PCT Filed: Jun. 30, 2022
(86) PCT No.: PCT/US2022/035858

§ 371 Date: Dec. 28, 2023

Related U.S. Application Data
(60) Provisional application No. 63/217,177, filed on Jun. 30, 2021.

(51) Int. Cl. C12N 15/86; C12N 9/22; C12N 15/113

(52) U.S. Cl. CPC C12N 15/86; C12N 9/22; C12N 15/113; C12N 2310/20; C12N 2310/531; C12N 2770/30043

(57) ABSTRACT

Described herein are engineered nodaviral vectors, systems, and uses thereof. In some embodiments, the engineered nodaviral vectors and/or systems can be used to deliver a cargo, such as a nucleic acid cargo, to a cell or cell population, or a subject. In some embodiments, the cargo is a therapeutic or preventative.

### CROSS-REFERENCE TO RELATED APPLICATIONS

This application claims the benefit of and priority to co-pending U.S. Provisional Patent Application No. 63/217,177, filed on Jun. 30, 2021, entitled ENGINEERED NODAVIRAL CARGO DELIVERY SYSTEMS,” the contents of which is incorporated by reference herein in its entirety.

### STATEMENT REGARDING FEDERALLY SPONSORED RESEARCH

This invention was made with government support under Grant No. 2022-67015-36332 awarded by USDA/NIFA. The government has certain rights in the invention.

### SEQUENCE LISTING

This application contains a sequence listing filed in electronic form as an ASCII.txt file entitled UAZ-ST25.txt, created on Jun. 29, 2022 and having a size of 41,152 bytes (45 KB on disk). The content of the sequence listing is incorporated herein in its entirety.

### TECHNICAL FIELD

The subject matter disclosed herein is generally directed to viral delivery vectors and systems.

### BACKGROUND

Viral diseases such as White Spot Disease (WSD), caused by the White Spot Syndrome Virus (WSSV) is a threat to crustacean (e.g., shrimp & crawfish) farming in the US and globally. WSD has caused cumulative losses of over $15 billion worldwide and it continues to cause large-scale mortalities periodically. The current approach to disease control is to stock ponds with genetically superior and SPF PLs (Specific Pathogen Free, Post Larvae), while implementing biosecurity measures on the farm are the cornerstones for preventing WSD. Most shrimp farmers practice intensive culture and stock their ponds with PLs at a high density (>150 PL/m²). As a result, these farms are highly susceptible to disease outbreak, in the case biosecurity is breached and WSD is introduced with no therapeutic or other prevention. Currently, there is no therapeutic to control a WSD outbreak and an emergency premature harvest remains as the only choice to prevent 100% crop loss for shrimp farmers worldwide when WSD induced mortality starts in a farm. While implementing biosecurity is beneficial in reducing the risk of WSD outbreak in a shrimp farm, it is difficult to control the disease once it gets introduced in a farm considering the virus has greater than 100 known hosts and there is no commercially available therapeutics and/or WSSV resistant line. Since crustaceans do not have an antibody-based adaptive immunity, conventional vaccine approaches, that have been widely successful in finfish aquaculture, are also not applicable. As such, there exists an urgent need for therapies and preventions for viral diseases in shrimp and other crustaceans.

Citation or identification of any document in this application is not an admission that such a document is available as prior art to the present invention.

### SUMMARY

Described in certain example embodiments herein are engineered cargo delivery system polynucleotides comprising an engineered gamma or beta nodavirus polynucleotide comprising a nodavirus capsid polypeptide construct comprising a gamma or beta nodavirus RNA2 encoding polynucleotide or a capsid forming domain thereof operatively coupled to one or more regulatory elements; and a cargo delivery construct comprising a 5′ untranslated region (UTR) of RNA1 of a native gamma or beta nodavirus, a 3′ UTR of RNA1 of a native gamma or beta nodavirus, or both; and a cargo polynucleotide, wherein the cargo polynucleotide is operatively coupled to the 5′ UTR of RNA1 of a native gamma or beta nodavirus, the 3′ UTR of RNA1 of a native gamma or beta nodavirus, or both, and wherein the cargo polynucleotide is operatively coupled to one or more regulatory elements.

In certain example embodiments, the engineered cargo delivery system polynucleotide does not contain a nodavirus RNA dependent polymerase encoding polynucleotide. In certain example embodiments, the engineered cargo delivery system polynucleotide does not contain any portion of the translated region of beta or gamma nodavirus RNA1.

In certain example embodiments, the nodavirus capsid polypeptide construct comprises a 5′ untranslated region (UTR) of a native gamma or beta nodavirus RNA 2, and a 3′ UTR of a native gamma or beta nodavirus RNA 2, or both, wherein the gamma or beta nodavirus RNA2 encoding polynucleotide or a capsid forming domain thereof is operatively coupled the 5′ UTR of a native gamma or beta nodavirus RNA 2, the 3′ UTR of a native gamma or beta nodavirus RNA 2, or both.

In certain example embodiments, the native gamma or beta nodavirus is selected from Macrobrachium rosenbergii nodavirus (MrNV), Penaeus vannamei nodavirus (PvNV), covert mortality nodavirus (CMNV), Farfantepenaeus duorarum nodavirus (FdNV), tiger puffer nervous necrosis virus (TPNNV), striped jack nervous necrosis virus (SJNNV), barfin flounder nervous necrosis virus (BFNNV), red-spotted grouper nervous necrosis virus (RGNNV), or an Egyptian Nile tilapia nervous necrosis virus (Egyptian NT NNV). In certain example embodiments, the native gamma nodavirus is MrNV.

In certain example embodiments, the engineered cargo delivery system polynucleotide comprises 1-10, 1-20, 1-30, 1-40, 1-50, 1-100, 1-150, 1-200, 1-250, 1-300, 1-350, 1-400, 1-500, 1-750, 1-1000, 1-1500, 1-2000, 1-2500, 1-3000, 1-3500 or more of the translated and/or untranslated region of the native gamma or beta nodavirus RNA2.

In certain example embodiments, the cargo polynucleotide encodes a protein, an RNA, or both. In certain example embodiments, the cargo encodes an RNA, wherein the RNA is not translated. In certain example embodiments, the cargo polynucleotide encodes an RNA, wherein the RNA is an interfering RNA, a gene silencing oligonucleotide, or a guide RNA for a CRISPR-Cas system.

In certain example embodiments, the cargo is a hairpin RNA, optionally a long hairpin RNA. In certain example embodiments, the one or more regulatory elements operatively coupled to the cargo polynucleotide comprises a Pol III promoter and optionally a Poll III termination sequence.

In certain example embodiments, the cargo polynucleotide encodes an RNA, wherein the RNA is an interfering RNA, a gene silencing oligonucleotide, or a guide RNA for a CRISPR-Cas system, and wherein the gene silencing oligonucleotide, the interfering RNA, and the guide RNA are capable of specifically hybridizing with or targeting a pathogenic DNA or RNA. In certain example embodiments, the pathogenic DNA or RNA is a pathogenic DNA or RNA from a shrimp, a prawn, and/or a fish pathogen. In certain example embodiments, the shrimp, prawn, or fish pathogen is a virus, a bacteria, a fungus, a worm, or a protozoan. In certain example embodiments, the shrimp prawn, or fish pathogen is white spot syndrome virus (WSSV), Yellow head virus (YHV), Gill associated virus (GAV), Infectious hypodermal and hematopoietic necrosis virus (IHHNV), Taura syndrome virus (TSV), Monodon baculovirus (MBV), Hepatopancreatic parvovirus (HPV), Related Australian lymphoid organ virus (LOV), Mourilyan virus (MOV), Laem Singh virus (LSNV), Baculovirus midgut gland necrosis virus (BMNV), Monodon slow growth syndrome (MSGS), Macrobrachium rosenbergii nodavirus (MrNV), Extra small virus (XSV), Spawner-isolated mortality virus (SMV), Lymphoidal parvo-like virus (LPV), Baculovirus penaei (BP), Shrimp iridovirus (SIV), Shrimp hemocyte iridescent virus (SHIV), Reo-like virus (REO) type II and IV, Lymphoid organ vacuolization virus (LOVV), Rhabdovirus of penaeid shrimp (RPS), Infectious myonecrosis virus (IMNV), a nodavirus, a bacteria of the genus Vibrio, a Rickettsiae, a fungs of the genus Langenidium, a fungus of the genus Sirolopidium, a fungus of the genus Fusarium, a protozoan of the genus Micropsoran, a protozoan of the genus Haplospora, a protozoan of the genus Gregarina, a trematode (e.g., of the family Opecoelidae, Microphalliadae, or Echinostomatidae), a cestode (e.g., of the genus Prochristianella, Parachristianella, or Renibulbus), a nematode (e.g., Spirocamallanus pereirai, Leptolaimus sp. Ascaropsis sp, and Hysterothylacium reliquens), Enterocytozoon hepatopenaei (EHP), Infectious myonecrosis virus, or infection with HPR-deleted or HPRO infectious salmon anemia virus (ISAV), Epizootic Haematopoietic Necrosis (EHN), Infectious Haematopoietic Necrosis (IHN), Infection with koi herpesvirus, Infection with red sea bream iridovirus, Infectious Spleen & Kidney Necrosis Virus (ISKNV), Infection with salmonid alphavirus, Oncorhynchus masou Virus (OMV), Infectious Pancreatic Necrosis (IPN), Viral Encephalopathy and Retinopathy (VER), Spring Viraemia of Carp (SVC), Channel Catfish Virus (CCV) Viral Haemorrhagic Septicaemia (VHS), Lymphocystis, Bacterial Kidney Disease (BKD), Epizootic Ulcerative Syndrome (EUS), Nervous Necrosis Virus (NNV), Tilapia Lake Virus (TiLV), or any combination thereof.

In certain example embodiments, the cargo delivery polynucleotide is a gene of interest or portion thereof. In certain example embodiments, the cargo delivery polynucleotide encodes a Cas protein or portion thereof.

Described in certain example embodiments herein are engineered vectors comprising an engineered cargo delivery system polynucleotide of the present disclosure as described herein. In certain example embodiments, the vector comprises one or more components of a baculoviral vector.

Described in certain example embodiments herein are engineered cargo delivery vector system comprising an engineered cargo delivery system polynucleotide of the present disclosure described herein or an engineered vector including the engineered cargo delivery system polynucleotide. In certain example embodiments, the cargo delivery construct and the engineered polynucleotide nodavirus capsid polypeptide construct are on the same vector. In certain example embodiments, the cargo delivery construct and the engineered polynucleotide nodavirus capsid polypeptide construct are on different vectors.

In certain example embodiments, the engineered cargo delivery vector or vector system thereof is capable of producing engineered nodavirus or nodaviral like particles. In certain example embodiments, the engineered cargo delivery vector or vector system thereof is capable of packaging the cargo polynucleotide into the engineered nodavirus or nodaviral like particles. In certain example embodiments, the engineered cargo delivery vector or vector system thereof and/or engineered nodavirus or nodaviral like particles produced therefrom is/are replication incompetent. In certain example embodiments, the cargo delivery construct and/or the nodavirus capsid polypeptide construct is/are included in a baculoviral vector.

Described in certain example embodiments herein are engineered nodavirus or nodavirus like particles produced from and/or containing an engineered cargo delivery system polynucleotide of the present disclosure described herein and/or engineered cargo delivery vector of or vector system thereof.

Described in certain example embodiments herein is a cell or population thereof comprising an engineered cargo delivery system polynucleotide of the present disclosure described herein, an engineered cargo delivery vector or vector system comprising the engineered cargo delivery system polynucleotide, an engineered nodavirus or nodavirus like particle or population thereof produced from the engineered cargo delivery system polynucleotide, and/or a formulation thereof.

In certain example embodiments, the cell or population thereof is a bacterial cell or population thereof or an insect cell or population thereof. In certain example embodiments, the cell or population thereof is non-pathogenic to shrimp, prawns, or other crustaceans, or fish, optionally a fin fish. In certain example embodiments, the cell or population thereof is capable of producing nodavirus or nodavirus like particles comprising the cargo polynucleotide.

Described in certain example embodiments herein are formulations comprising an engineered cargo delivery system polynucleotide of claim of the present disclosure described herein, an engineered cargo delivery vector or vector system thereof comprising the engineered cargo delivery system polynucleotide, a cell or population thereof comprising the engineered cargo delivery system polynucleotide, an engineered nodavirus or virus like particle or population produced by or comprising the engineered delivery cargo delivery system polynucleotide, or any combination thereof.

In certain example embodiments, the formulation is a feed formulation and is optionally adapted for shrimp, prawns, or another crustacean, or fish, optionally a fin fish.

Described in certain example embodiments are methods of producing engineered gamma or beta nodavirus or nodavirus like particles carrying a cargo expressing an engineered cargo delivery system polynucleotide of the present disclosure described herein, an engineered cargo delivery vector or vector system comprising the engineered cargo delivery system polynucleotide in a cell capable of expressing the engineered cargo delivery system polynucleotide, vector, vector system, or any combination thereof.

Described in certain example embodiments is an engineered nodavirus or nodavirus like particle or population thereof produced by the method of the present disclosure described herein.

Described in certain example embodiments are methods of delivering a cargo to a recipient cell or cell population, the method comprising delivering an engineered cargo delivery system polynucleotide of the present disclosure described herein to the recipient cell or cell population, a vector or vector system comprising the engineered cargo delivery system polynucleotide, and/or an engineered nodavirus or virus like particle produced by or containing the engineered cargo delivery system polynucleotide to a subject comprising the recipient cell or cell population and/or to the recipient cell of cell population.

In certain example embodiments, the engineered cargo delivery system polynucleotide of the present disclosure described herein to the recipient cell or cell population, a vector or vector system comprising the engineered cargo delivery system polynucleotide, and/or an engineered nodavirus or virus like particle produced by or containing the engineered cargo delivery system polynucleotide is contained in a formulation. In certain example embodiments, the formulation is a feed formulation, optionally formulated for shrimp, prawn or other crustacean or a fish, optionally a fin fish. In certain example embodiments, the subject is a shrimp, prawn, other crustacean, or fish, optionally a fin fish.

Described in certain example embodiments herein are methods of treating and/or preventing a disease in a crustacean or a fish, optionally a fin fish, comprising delivering to the crustacean or fish or a cell thereof an engineered cargo delivery system polynucleotide of the present disclosure described herein to the recipient cell or cell population, a vector or vector system comprising the engineered cargo delivery system polynucleotide, and/or an engineered nodavirus or virus like particle produced by or containing the engineered cargo delivery system polynucleotide.

In certain example embodiments, the engineered cargo delivery system polynucleotide of the present disclosure described herein to the recipient cell or cell population, a vector or vector system comprising the engineered cargo delivery system polynucleotide, and/or an engineered nodavirus or virus like particle produced by or containing the engineered cargo delivery system polynucleotide is contained in a formulation. In certain example embodiments, the formulation is a feed formulation, optionally formulated for shrimp, prawn or other crustacean or a fish, optionally a fin fish.

In certain example embodiments, the disease is caused by a pathogen, wherein the pathogen is white spot syndrome virus (WSSV), Yellow head virus (YHV), Gill associated virus (GAV), Infectious hypodermal and hematopoietic necrosis virus (IHHNV), Taura syndrome virus (TSV), Monodon baculovirus (MBV), Hepatopancreatic parvovirus (HPV), Related Australian lymphoid organ virus (LOV), Mourilyan virus (MOV), Laem Singh virus (LSNV), Baculovirus midgut gland necrosis virus (BMNV), Monodon slow growth syndrome (MSGS), Macrobrachium rosenbergii nodavirus (MrNV), Extra small virus (XSV), Spawner-isolated mortality virus (SMV), Lymphoidal parvo-like virus (LPV), Baculovirus penaei (BP), Shrimp iridovirus (SIV), Shrimp hemocyte iridescent virus (SHIV), Reo-like virus (REO) type II and IV, Lymphoid organ vacuolization virus (LOVV), Rhabdovirus of penaeid shrimp (RPS), Infectious myonecrosis virus (IMNV), a nodavirus, a bacteria of the genus Vibrio, a Rickettsiae, a fungs of the genus Langenidium, a fungus of the genus Sirolopidium, a fungus of the genus Fusarium, a protozoan of the genus Micropsoran, a protozoan of the genus Haplospora, a protozoan of the genus Gregarina, a trematode (e.g., of the family Opecoelidae, Microphalliadae, or Echinostomatidae), a cestode (e.g., of the genus Prochristianella, Parachristianella, or Renibulbus), a nematode (e.g., Spirocamallanus pereirai, Leptolaimus sp. Ascaropsis sp, and Hysterothylacium reliquens), Enterocytozoon hepatopenaei (EHP), Infectious myonecrosis virus, or infection with HPR-deleted or HPRO infectious salmon anemia virus (ISAV), Epizootic Haematopoietic Necrosis (EHN), Infectious Haematopoietic Necrosis (IHN), Infection with koi herpesvirus, Infection with red sea bream iridovirus, Infectious Spleen & Kidney Necrosis Virus (ISKNV), Infection with salmonid alphavirus, Oncorhynchus masou Virus (OMV), Infectious Pancreatic Necrosis (IPN), Viral Encephalopathy and Retinopathy (VER), Spring Viraemia of Carp (SVC), Channel Catfish Virus (CCV) Viral Haemorrhagic Septicaemia (VHS), Lymphocystis, Bacterial Kidney Disease (BKD), Epizootic Ulcerative Syndrome (EUS), Nervous Necrosis Virus (NNV), Tilapia Lake Virus (TiLV), or any combination thereof.

Described in certain example embodiments herein are kits comprising an engineered cargo delivery polynucleotide of the present disclosure described herein, an engineered cargo delivery vector or vector system of the present disclosure described herein, a cell or population thereof of the present disclosure described herein, an engineered nodavirus or virus like particle of the present disclosure described herein, a formulation of the present disclosure described herein, or any combination thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—A schematic overview of developing oral vaccines/therapeutics against diseases in aquatic animals using a Nodavirus-based viral vector system.

FIG. 2—A schematic representation of the genome organization of Macrobrachium rosenbergii nodavirus (MrNV) and extra small virus (XSV) infecting freshwater prawn and penaeid shrimp. The numbers above the lines represent nucleotide numbers of the viral RNA. The box indicates an open reading frame encoding RNA-dependent RNA polymerase (RdRp) in RNA1 (nucleotides 24 to 3161) and capsid protein (CP) gene (nucleotides 38 to 1153) in RNA2 segment of the viral genome. The genome of XSV encodes a capsid protein only (nucleotides 89 to 613).

FIG. 3—A genomic map of infectious cDNA clones of MrNV and XSV. The full-length RNA1 and RNA 2 of MrNV (Left Panel) and full-length genome of XSV were clones in a baculovirus pFastBacDUAL vector (Gangnonngiw et al., Virology 2020, 540, 30-37).

FIG. 4—Transmission electron micrographs of negatively stained viral preparations of MrNV (left), XSV (middle) and recombinant baculovirus (right) purified from Sf9 cells (Gangnonngiw et al., 2020).

FIG. 5—A genomic map of infectious recombinant MrNV where the open reading frame (ORF) representing the RNA-dependent RNA polymerase (RdRp) gene in RNA1 was replaced with a marker gene, Green Fluorescent Protein (GFP) gene. In the bottom of the panel, a schematic representation of the GFP gene construct is shown. The numbers on the schematic represent nucleotide number. For example, in MrNV RNA 1, nucleotides 1 to 23 represent the 5′-end non-coding region, nucleotides 24 to 1295 represent GFP ORF and nucleotides 1295 through 1336 indicate 3′-end non-coding region of MrNV RNA1. In RNA 2, nucleotides 1 to 37 represent the 5′-end non-coding region, nucleotides 38 to 1153 represent GFP ORF and nucleotides 1154 to 1175 indicate the 3′-end non-coding region of the RNA.

FIG. 6—GFP expression in SF9 cells 5 days post-inoculation with rBV_MrNV-GFP. Upper panel showing bright field images (BF) and on the lower, GFP-positive cells. Scale bars represent 200 μm.

FIG. 7—A genomic map of infectious cDNA clones of sevenband grouper nervous necrosis virus (SGNNV). The full-length RNA1 and RNA 2 of SGNNV were cloned under the control of polyhedrin and p10 promoters, respectively in a baculovirus pFastBacDUAL vector. Recombinant baculovirus carrying SGNNV genome is used to infect Sf9 cells to produce infectious cDNA clones of SGNNV.

FIG. 8—A map of a recombinant MrNV as a viral vector to deliver a foreign gene in shrimp cells. MrNV RNA1 ORF was replaced by green fluorescent protein (GFP) gene. In the genomic map of infectious MrNV clone containing full-length RNA1 and RNA 2 in a baculovirus pFastBacDUAL vector (Left Panel) and a recombinant MrNV, the RdRp ORF in RNA1 is replaced by GFP gene (Right Panel).

FIG. 9—Flow cytometry analysis demonstrating efficiency of rBV-MrNV-GFP in delivering GFP gene to SF9 cells. Only single cell events were analyzed as multiplicity results to autofluorescence. Non-inoculated cells were run as the baseline control (left) and two days post-inoculated cells were analyzed for GFP expression (right) where positive cells were found to be 98% of the total number of cells population.

FIG. 10—Delivery of vector and expression of an exemplary gene payload. Bright field (BF) and fluorescent (GFP) microscope images of P. vannamei hemocytes drawn after 5 days of consecutive feeding with feeds soaked in SF9 cells infected with MrNV-GFP_BV (oral); and 5 days post-intramuscular inoculation of MrNV-GFP_BV (injection). Naïve/specific pathogen free (SPF) shrimp were used as negative control. Scale bar indicated is at 10 μm.

FIG. 11—Exemplary strategy for evaluating the efficacy of engineered nodaviral vectors delivering a therapeutic/preventative RNA or other cargo delivered by an oral route in preventing disease (e.g., WSD in P. vannamei shrimp).

FIG. 12—Screenshot demonstrating open reading frame and non-coding region in silico analysis of MrNV-RNA-1 inserted downstream of a Pph promoter (SEQ ID NO: 11).

FIG. 13—Screenshot demonstrating open reading frame and non-coding region in silico analysis of MrNV-RNA-2 inserted downstream of a P10 promoter (SEQ ID NO: 12).

FIG. 14—Screenshot demonstrating open reading frame and non-coding region in silico analysis of MrNV RNA-1 containing GFP sequence-downstream of Pph promoter (SEQ ID NO: 13).

FIG. 15—Exemplary MrNVcp-GFP Viral Vector Construct. Machrobrachium rosenbergii nodavirus (MrNV) was reengineered into a replication-deficient viral vector “MrNVcp” by replacing RNA-1 ORF encoding RNA-dependent RNA polymerase (RdRp) with the green fluorescent protein (GFP), while retaining RNA-1 UTR region and RNA-2 encoding for the capsid protein (CP). These two fragments are co-expressed in a dual expression system using pFastBac Dual vector using baculovirus in insect Spodoptera frugiperda Sf9 cells line. The resulting inoculum, comprising of baculovirus (BV) expressing MrNVcp vector carrying GFP, shall be referred to as BV_MrNVcp-GFP.

FIG. 16—Flow cytometry analysis of Sf9 cells infected with BV_MrNVcp-GFP. Population of GFP-expressing SF9 cells in both uninfected (upper panel) and infected (lower panel) 92 hpi with 1:1 MOI of BV_MrNV-GFP. Number of events (#Events) and percentage of GFP-expressing cells (% Parent) are graphed below the graph plots.

FIG. 17A-17C—Production of MrNV-GFP in SF9 cells by a baculovirus expression system. Transmission Electron Microscopy (TEM) images of ultra-thin sections of SF9 cells infected with baculovirus expressing MrNV-GFP. Micrographs of lysed SF9 cells showing occlusion bodies (black arrow); scale bar=2 μm (FIG. 17A). Higher resolution focusing on baculovirus (white arrow) and MrNV-containing vesicles (arrowheads); scale bar=500 nm (FIG. 17B). MrNV viral particles measured about 30-40 nm; scale bar=200 nm (FIG. 17C).

FIG. 18A-18C—Separation of Baculovirus and MrNVcp-GFP. Negative stained TEM images of the soluble fraction of the inoculum containing a mixture of the recombinant baculovirus (white arrows) and the expressed MrNVcp-GFP (A) viral vector; scale bar (a). The two particles were separated by density-gradient ultracentrifugation show rod-shaped baculovirus nucleocapsids (b) and MrNVcp-GFP particles with average size of 40 nm. Scale bars are set to 200 nm.

FIG. 19A-19B—Delivery route and MrNVcp Vector Detection. (FIG. 19A) Shows experimental strategy for evaluating delivery route efficacy. (FIG. 19B) RT-PCR detection of MrNV capsid protein (CP) gene after injecting or feeding specific pathogen free (SPF) Penaeus vannamei shrimp with BV_MrNV-GFP. Delivery by feeding was conducted by feeding with feeds soaked with inoculum containing Sf9 cells infected with BV_MrNV-GFP. 5 days post-injection (injection) and after 5 days of consecutive feeding with feeds soaked in SF9 cells infected with BV_MrNV-GFP (feeding). Naïve/Specific Pathogen Free (SPF) shrimp were used as negative control.

FIG. 20A-20B—Quantity and Lifespan of Orally-Delivered MrNVcp. Absolute quantification of MrNVcp vector measured using real-time RT-PCR of the capsid protein RNA. SPF P. vannamei shrimp were fed with diet containing MrNVcp-GFP expressed in Sf9 cells for 5 consecutive days and animals (n=3) were sacrificed at 0 (5th feeding day), 1, 3, 5 and 8 days post-delivery of vector. Copy number of MrNVcp RNA was measured and quantified by real-time RT-PCR in hepatopancreas (FIG. 20A) and pleopods (FIG. 20B) using MrNV RNA dilution standards. RNA isolated from respective tissues of shrimp injected with BV_MrNVcp-GFP 5 days post-injection are shown on the right side as injection control.

FIG. 21A-21D—MrNVcp Tropism in Shrimp Hemocyte Cells. Transmission electron microscopy (TEM) images of ultra-thin sections of hemocytes drawn from Penaeus vannamei fed with treatment diet containing commercial feeds soaked in the inoculum solution (Sf9 cells infected with BV_MrNVcp-GFP). The treatment diet were fed for 5 consecutive days, once per day at 5% biomass. Micrographs showing MrNV particles in areas of hemocytes cells marked by a white box; scale bar=2 μm (FIG. 21A and FIG. 21C). Higher resolution focusing on MrNVcp particles measuring ˜30-40 nm are indicated by white triangle (A); scale bar=1 μm (FIG. 21B) and 200 nm (FIG. 21D).

FIG. 22—Expression of green fluorescent protein in Sf9 cells. Bright field and fluorescent images of Sf9 cells infected with BV_MrNVcp-GFP at 1:1, 1:10, and 1:100 multiplicity of infection (MOI) observed at 24, 48, and 92 hours post-inoculation (hpi). (Scale bar=200 μm).

FIG. 23—Primers used in this study for RT-PCR and real-time qRT-PCR. Primers and probes were designed using the Primer Express® Software 3.0 (Applied Biosystems, Warrington, UK) (SEQ ID NOs: 14-21).

FIG. 24—Shows a production, validation, and delivery scheme for oral delivery of a nodavirus vector capable of producing interfering RNA or other therapeutic nucleic acid to an animal, such as a crustacean.

FIG. 25A-28B—Shows an exemplary strategy for Pol II transcription of a reporter gene (FIG. 25A (SEQ ID NO: 22)) or a RNAi molecule (FIG. 25B (SEQ ID NOs: 22-23)) in in an embodiment of the nodaviral vector of the present description.

FIG. 26—Shows secondary structure(s) produced from Pol II transcription of a long hairpin molecule in an embodiment of the nodaviral vector of the present description (SEQ ID NO: 24).

FIG. 27—Shows an exemplary strategy for Pol III transcription of a therapeutic nucleic acid, e.g., a RNAi molecule such as a long hairpin RNA in an embodiment of the nodaviral vector of the present description (SEQ ID NO: 23).

FIG. 28—Shows a scheme for an hRNA (e.g., lhRNA) treatment and/or vaccine for a shrimp pathogenic virus, WSSV (SEQ ID NO: 23).

FIG. 29—Shows exemplary lhRNA target design against a WSSV VP28 structural protein for an hRNA targeting WSSV to be delivered via the strategy shown in FIG. 28 (SEQ ID NOs: 25-26).

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, ‘less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracomeal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or poly deoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct) or other interfering RNA molecule or a gene silencing oligonucleotide, siRNA (short interfering RNA), microRNA (miRNA), short hairpin RNA, long non-coding RNA, or ribozymes, aptamers, guide RNA (gRNA) or coding mRNA (e.g., messenger RNA).

As used herein, the terms “disease” or “disorder” are used interchangeably throughout this specification and refer to any alternation in state of the body or of some of the organs, interrupting or disturbing the performance of the functions and/or causing symptoms such as discomfort, dysfunction, distress, or even death to the subject afflicted or those in contact with a person. A disease or disorder can also be related to a distemper, ailing, ailment, malady, disorder, sickness, illness, complaint, indisposition, or affliction.

As used herein, “dose,” “unit dose,” or “dosage” can refer to physically discrete units suitable for use in a subject, each unit containing a predetermined quantity of the e.g., virus like particles, cells, vectors, vector systems, or other composition described herein and/or a pharmaceutical formulation thereof calculated to produce the desired response or responses in association with its administration.

As used herein, “fragment” or “portion” as used throughout this specification with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300, ≥400, ≥500 or ≥600 consecutive amino acids of the corresponding full-length peptide, polypeptide, or protein. The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a non-translated catalytic RNA molecule, including but not limited to, tRNA, siRNA, piRNA, miRNA, long-non-coding RNA and shRNA.

As used herein, the terms “guide polynucleotide,” “guide sequence,” or “guide RNA” as can refer to any polynucleotide sequence having sufficient complementarity with a target polynucleotide sequence to hybridize with the target sequence and direct sequence-specific binding of a CRISPR complex to the target sequence. The degree of complementarity between a guide polynucleotide and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif), SOAP (available at soap.genomics.org.cn), and Maq (available at maq.sourceforge.net). A guide polynucleotide can be about or more than about 5, 10, 11, 12, 13, 14, 15, 16, 17, 18, 19, 20, 21, 22, 23, 24, 25, 26, 27, 28, 29, 30, 35, 40, 45, 50, 75, 90, 100, 110, 112, 115, 120, 130, 140, or more nucleotides in length. The guide polynucleotide can include a nucleotide sequence that is complementary to a target DNA or RNA sequence.

A guide polynucleotide can be less than about 150, 125, 75, 50, 45, 40, 35, 30, 25, 20, 15, 12, or fewer nucleotides in length. The ability of a guide polynucleotide to direct sequence-specific binding of a CRISPR complex to a target sequence may be assessed by any suitable assay. For example, the components of a CRISPR system sufficient to form a CRISPR complex, including the guide polynucleotide to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

A complementary region of the gRNA can be configured to target any DNA region of interest. The complementary region of the gRNA and the gRNA can be designed using a suitable gRNA design tool. Suitable tools are known in the art and are available to the skilled artisan. As such, the constructs described herein are enabled for any desired target DNA so long as it is CRISPR compatible according to the known requirements for CRISPR activation.

A guide polynucleotide can be selected to reduce the degree of secondary structure within the guide polynucleotide. Secondary structure may be determined by any suitable polynucleotide folding algorithm. Some programs are based on calculating the minimal Gibbs free energy. An example of one such algorithm is mFold, as described by Zuker & Stiegler ((1981) Nucleic Acids Res. 9, 133-148). Another example folding algorithm is the online webserver RNAfold, developed at Institute for Theoretical Chemistry at the University of Vienna, using the centroid structure prediction algorithm (see e.g. Gruber et al., (2008) Cell 106: 23-24; and Carr & Church (2009) Nature Biotechnol. 27: 1151-1162).

As used herein, “gene silencing oligonucleotide” refers to any oligonucleotide that can alone or with other gene silencing oligonucleotides utilize a cell's endogenous mechanisms, molecules, proteins, enzymes, and/or other cell machinery or exogenous molecule, agent, protein, enzyme, and/or polynucleotide to cause a global or specific reduction or elimination in gene expression, RNA level(s), RNA translation, RNA transcription, that can lead to a reduction or effective loss of a protein expression and/or function of a non-coding RNA as compared to wild-type or a suitable control. This is synonymous with the phrase “gene knockdown” Reduction in gene expression, RNA level(s), RNA translation, RNA transcription, and/or protein expression can range from about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, to 1% or less reduction. “Gene silencing oligonucleotides” include, but are not limited to, any antisense oligonucleotide, ribozyme, any oligonucleotide (single or double stranded) used to stimulate the RNA interference (RNAi) pathway in a cell (collectively RNAi oligonucleotides), small interfering RNA (siRNA), microRNA, short-hairpin RNA (shRNA), and gRNAs for CRISPR. Commercially available programs and tools are available to design the nucleotide sequence of gene silencing oligonucleotides for a desired gene, based on the gene sequence and other information available to one of ordinary skill in the art.

As used herein, “infection” as used herein refers to presence of an infective agent, such as a pathogen, e.g., a microorganism, in or on a subject, which, if its presence or growth were inhibited, would result in a benefit to the subject. Hence, the term refers to the state produced by the establishment, more particularly invasion and multiplication, of an infective agent, such as a pathogen, e.g., a microorganism, in or on a suitable host. An infection may produce tissue injury and progress to overt disease through a variety of cellular and toxic mechanisms.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used interchangeably herein, “operatively linked” and “operably linked” in the context of recombinant or engineered polynucleotide molecules (e.g. DNA and RNA) vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g. promoters, enhancers, and termination elements), and/or selectable markers in an expression vector. “Operatively linked” can also refer to an indirect attachment (i.e. not a direct fusion) of two or more polynucleotide sequences or polypeptides to each other via a linking molecule (also referred to herein as a linker).

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, crustaceans (e.g., shrimp and prawns), fish, mammals, e.g., cats, dogs, horses, pigs, cows, sheep, rodents, rabbits, squirrels, bears, primates (e.g., chimpanzees, gorillas, and humans).

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “plasmid” refers to a non-chromosomal double-stranded DNA sequence including an intact “replicon” such that the plasmid is replicated in a host cell.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

As used herein, “promoter” includes all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used herein, the terms “treating”, and “treatment” refer generally to obtaining a desired pharmacological and/or physiological effect. The effect can be, but does not necessarily have to be, prophylactic in terms of preventing or partially preventing a disease, symptom or condition thereof, such as an infection by a pathogen or disease therefrom. The effect can be therapeutic in terms of a partial or complete cure of a disease, condition, symptom or adverse effect attributed to the disease, disorder, or condition. The term “treatment” as used herein covers any treatment of infection by a pathogen or disease therefrom, in a subject, particularly a crustacean or a fish, particularly a fin fish, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the term “vector” or is used in reference to a vehicle used to introduce an exogenous nucleic acid sequence into a cell. A vector may include a DNA molecule, linear or circular (e.g., plasmids), which includes a segment encoding an RNA and/or polypeptide of interest operatively linked to additional segments that provide for its transcription and optional translation upon introduction into a host cell or host cell organelles. Such additional segments can include promoter and/or terminator sequences, and can also include one or more origins of replication, one or more selectable markers, an enhancer, a polyadenylation signal, etc. Expression vectors are generally derived from yeast or bacterial genomic or plasmid DNA, or viral DNA, or may contain elements of both. Expression vectors can be adapted for expression in prokaryotic or eukaryotic cells. Expression vectors can be adapted for expression in mammalian, fungal, yeast, or plant cells. Expression vectors can be adapted for expression in a specific cell type via the specific regulator or other additional segments that can provide for replication and expression of the vector within a particular cell type.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

As used herein, “native” refers to the natural form of an organism.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

As used herein, “Cas protein” or “Cas polypeptide” refers to a Cas protein or polypeptide, such as a Class 1 or Class 2 Cas protein that is capable of use within a CRISPR-Cas system, such as Cas 9, or other Cas protein generally known in the art.

All publications, published patent documents, and patent applications cited herein are hereby incorporated by reference to the same extent as though each individual publication, published patent document, or patent application was specifically and individually indicated as being incorporated by reference.

### Overview

Viral diseases such as White Spot Disease (WSD), caused by the White Spot Syndrome Virus (WSSV) is a threat to crustacean (e.g., shrimp & crawfish) farming in the US and globally. WSD has caused cumulative losses of over $15 billion worldwide and it continues to cause large-scale mortalities periodically. Currently, there is no measure to control the disease except prevention. While implementing biosecurity is beneficial in reducing the risk of WSD outbreak in a shrimp farm, it is difficult to control the disease once it gets introduced in a farm considering the virus has >100 known hosts and there is no commercially available therapeutics and/or WSSV resistant line. Since crustaceans do not have an antibody-based adaptive immunity, conventional vaccine approaches, that have been widely successful in finfish aquaculture, are also not applicable. RNA interference (RNAi)-based therapeutics delivered through injection to live shrimp to control WSD has shown promise in the laboratory. However, due to the lack of an oral delivery method, RNAi approach has not been translated to control WSD or other viral pathogens for a commercial application since injection of shrimp to deliver therapeutic molecule is not feasible.

With that said, embodiments disclosed herein can provide viral vectors and systems for delivery of nucleic acids to shrimp and other crustaceans, as well as fish, particularly fin fish. In some embodiments, the viral vectors and systems are recombinant Nodaviral vectors. In some embodiments, the vectors and/or systems thereof can provide an oral viral preventive and/or therapy. In some embodiments, the vectors and/or systems thereof can be effective to prevent and/or treat a viral infection after oral administration. In some embodiments, the Nodaviral vectors and/or systems include and can deliver an RNAi molecule capable of treating and/or preventing e.g., a viral infection a shrimp and/or other crustaceans. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Engineered Nodaviral Vectors and Systems

Described herein are engineered nodaviral vectors and systems that are based in part on crustacean or fish nodavirus that can be capable of delivering cargo polynucleotides via replication incompetent nodavirus to a recipient cell or cell population.

Nodavirus belongs to the family of Nodaviridae. In general, nodaviruses are non-enveloped zoonotic viruses with icosahedral structures. Their genomes comprise of two linear, positive-sense, single-stranded RNA. RNA 1 is approximately 3.1-3.2 kilobases (kb) in length, whereas RNA2 is approximately 1.2-1.4 kb. Both of which lack a poly-A tail at their 3′ ends (Comps et al., Aquaculture. 1994; 123:1-10. doi: 10.1016/0044-8486(94)90114-7 and Mori et al., Virology. 1992 March; 187(1):368-71). RNA 1 encodes for the RNA-dependent RNA polymerase (RdRP), which functions in replicating the viral RNA genome without involving an intermediate DNA. RNA 3, a subgenomic transcript of RNA 1, it encodes for a non-structural B2-like protein (Cai et al., Virus Res. 2010 August; 151(2):153-61, Hayakijkosol and Owens. Aquaculture. 2012; 326-329:40-45. doi: 10.1016/j.aquaculture.2011.11.023, and Lingel et al., EMBO Rep. 2005 December; 6(12):1149-55. B2 functions as a suppressor for the post-transcriptional gene silencing of host defense mechanisms through non-specific binding to double-stranded RNA generated during the virus replication (Fenner et al., J Virol. 2006 January; 80(1):85-94). RNA 2 encodes for the viral capsid protein, which forms the core of nodavirus. The nodavirus capsid protein assembles into virus particles with icosahedral structures, approximately 30 nm in diameter, with a triangulation number of 3 (T=3) containing 180 capsid subunits. The virus particles package only the RNA 1 and RNA 2, forming simple but infectious virions.

Nodaviruses are generally classified into alpha-nodaviruse and beta-nodaviruse based on their hosts. Alpha-nodaviruses generally infect insects (Schuster et al. J Virol. 2014 Nov; 88(22):13447-59), while beta-nodaviruses generally infect fishes, particularly fin fishes. A third type, gamma nodavriuses, of nodavirus primarily infects prawns and other crustaceans (Naveen Kumar et al., Virus Research. 2013; 173:377-385. doi: 10.1016/j.virusres.2013.01.003). Fish nodavirus is also known as Nervous Necrosis Virus (NNV). Exemplary fish infecting nodaviruses include tiger puffer nervous necrosis virus (TPNNV), striped jack nervous necrosis virus (SJNNV), barfin flounder nervous necrosis virus (BFNNV), red-spotted grouper nervous necrosis virus (RGNNV) (see e.g., Costa J Z, Thompson K D, Fish Shellfish Immunol. 2016 June; 53( ):35-49), and an Egyptian Nile tilapia nervous necrosis virus (Egyptian NT NNV) (see e.g., Taha et al. Aquaculture International (2020) 28:1811-1823). To date, nodavirus is known to affect over 120 fish species, particularly groupers and seabass such as the Asian seabass Lates calcarifer and European seabass Dicentrarchus labrax (see e.g., Breuil et al. (1991) 97:109-116; Costa J Z, Thompson K D, Fish Shellfish Immunol. 2016 June; 53( ):35-49; Frerichs G N, Rodger H D, Peric Z. J Gen Virol. 1996 Sep; 77 (Pt 9)( ):2067-71; Munday, Kwang & Moody (2002) Munday B L, Kwang J, Moody N. Betanodavirus infections of teleost fish: a review. Journal of Fish Diseases. 2002; 25:127-142; Parameswaran et al. (2008) Parameswaran V, Kumar S R, Ahmed V PI, Sahul Hameed A S. A fish nodavirus associated with mass mortality in hatchery-reared Asian Seabass, Lates calcarifer. Aquaculture. 2008; 275:366-369; and Parameswaran et al. (2008) Parameswaran V, Kumar S R, Ahmed V PI, Sahul Hameed A S. A fish nodavirus associated with mass mortality in hatchery-reared Asian Seabass, Lates calcarifer. Aquaculture. 2008; 275:366-369). Recently, large-scale mortalities in Egyptian hatchery-reared Nile tilapia (Oreochromis niloticus) due to a nervous necrosis virus has been reported (Taha et al., 2020). Phylogenetic analyses based on capsid protein gene sequence revealed that viral nervous necrosis causing mortalities in Nile tilapia is related to red-spotted grouper NNV (RGNNV) (Taha et al., 2020).

Exemplary prawn/crustacean infecting nodaviruses include Macrobrachium rosenbergii nodavirus (MrNV), Penaeus vannamei nodavirus (PvNV), covert mortality nodavirus (CMNV) (see e.g., Zhang Q, et al. J Gen Virol. 2014 December; 95(Pt 12):2700-2709), and Farfantepenaeus duorarum nodavirus (FdNV) (see e.g., Ng et al. Dis Aquat Organ. 2013 Sep. 3; 105(3):237-42). Although nodaviruses are traditionally named after their native hosts, they often are capable of infecting multiple species. For example, MrNV has also been reported to infect Penaeus indicus, Penaeus monodon, and P. vannamei (see e.g., Ravi et al., Aquaculture. 2009; 292:117-120. doi: 10.1016/j.aquaculture.2009.03.051 and Senapin et al., Aquaculture. 2012; 338-341:41-46. doi: 10.1016/j.aquaculture.2012.01.019).

MrNV was first isolated and reported in 1999 (see e.g., Arcier et al., 1999; 38:177-181. doi: 10.1186/s40064-016-3127-z) from M. rosenbergii. Infection by MrNV causes white tail disease (WTD) or white muscle disease (WMD), where infected cells undergo necrosis and turn whitish. The rate of mortality is extremely high (up to 100%) in larvae and post-larvae of M. rosenbergii (see e.g., Qian et al., J Fish Dis. 2003 September; 26(9):521-7 and Ravi et al. Aquaculture. 2009; 292:117-120. doi: 10.1016/j.aquaculture.2009.03.051) causing great economic losses to M rosenbergii hatchery and nursery farm industries. Despite the high mortality rate in larvae and post-larvae prawns, MrNV does not cause death in adult prawns. However, the adult prawns still serve as the virus carriers, transmitting the virus vertically to their offspring (see e.g., Sudhakaran et al., J Fish Dis. 2007 January; 30(1):27-35) and horizontally to other prawns during cannibalization (Sahul Hameed et al., Dis Aquat Organ. 2004 Dec. 13; 62(3):191-6).

Another prawn virus, PvNV was first isolated in 2005 from a P. vannamei farm in Belize (see e.g., Tang et al. Dis Aquat Organ. 2007 May 9; 75(3):183-90 and Tang et al., Dis Aquat Organ. 2011 May 9; 94(3):179-87). Being a prawn nodavirus, PvNV shares 83% similarities with MrNV in its viral genome (see e.g., Tang et al., Dis Aquat Organ. 2011 May 9; 94(3):179-87). It causes muscle necrosis, resulting in white, opaque lesions in the tail, similar to the symptoms of MrNV infection. However, the virulence of PvNV is not as high as MrNV, in which the former normally resulted in approximately 50% production loss in an infected farm (Tang et al. Dis Aquat Organ. 2007 May 9; 75(3):183-90). Apart from its native host, PvNV has also been demonstrated to be able to infect Penaeus monodon in an experimental infection (Tang et al. Dis Aquat Organ. 2007 May 9; 75(3):183-90).

Described in certain example embodiments herein are engineered vectors comprising an engineered cargo delivery system polynucleotide of the present disclosure as described herein. In certain example embodiments, the vector comprises one or more components of a baculoviral vector.

Described in certain example embodiments herein are engineered cargo delivery vector system comprising an engineered cargo delivery system polynucleotide of the present disclosure described herein or an engineered vector including the engineered cargo delivery system polynucleotide. In certain example embodiments, the cargo delivery construct and the engineered polynucleotide nodavirus capsid polypeptide construct are on the same vector. In certain example embodiments, the cargo delivery construct and the engineered polynucleotide nodavirus capsid polypeptide construct are on different vectors.

In certain example embodiments, the engineered cargo delivery vector or vector system thereof is capable of producing engineered nodavirus or nodaviral like particles. In certain example embodiments, the engineered cargo delivery vector or vector system thereof is capable of packaging the cargo polynucleotide into the engineered nodavirus or nodaviral like particles. In certain example embodiments, the engineered cargo delivery vector or vector system thereof and/or engineered nodavirus or nodaviral like particles produced therefrom is/are replication incompetent. In certain example embodiments, the cargo delivery construct and/or the nodavirus capsid polypeptide construct is/are included in a baculoviral vector.

Described in certain example embodiments herein are engineered cargo delivery system polynucleotides comprising an engineered gamma or beta nodavirus polynucleotide comprising a nodavirus capsid polypeptide construct comprising a gamma or beta nodavirus RNA2 encoding polynucleotide or a capsid forming domain thereof operatively coupled to one or more regulatory elements; and a cargo delivery construct comprising a 5′ untranslated region (UTR) of RNA1 of a native gamma or beta nodavirus, a 3′ UTR of RNA1 of a native gamma or beta nodavirus, or both; and a cargo polynucleotide, wherein the cargo polynucleotide is operatively coupled to the 5′ UTR of RNA1 of a native gamma or beta nodavirus, the 3′ UTR of RNA1 of a native gamma or beta nodavirus, or both, and wherein the cargo polynucleotide is operatively coupled to one or more regulatory elements.

In certain example embodiments, the engineered cargo delivery system polynucleotide does not contain a nodavirus RNA dependent polymerase encoding polynucleotide. In certain example embodiments, the engineered cargo delivery system polynucleotide does not contain any portion of the translated region of beta or gamma nodavirus RNA1.

In certain example embodiments, the nodavirus capsid polypeptide construct comprises a 5′ untranslated region (UTR) of a native gamma or beta nodavirus RNA 2, and a 3′ UTR of a native gamma or beta nodavirus RNA 2, or both, wherein the gamma or beta nodavirus RNA2 encoding polynucleotide or a capsid forming domain thereof is operatively coupled the 5′ UTR of a native gamma or beta nodavirus RNA 2, the 3′ UTR of a native gamma or beta nodavirus RNA 2, or both.

In certain example embodiments, the native gamma or beta nodavirus is selected from Macrobrachium rosenbergii nodavirus (MrNV), Penaeus vannamei nodavirus (PvNV), covert mortality nodavirus (CMNV), Farfantepenaeus duorarum nodavirus (FdNV), tiger puffer nervous necrosis virus (TPNNV), striped jack nervous necrosis virus (SJNNV), barfin flounder nervous necrosis virus (BFNNV), red-spotted grouper nervous necrosis virus (RGNNV), or an Egyptian Nile tilapia nervous necrosis virus (Egyptian NT NNV). In certain example embodiments, the native gamma nodavirus is MrNV.

In certain example embodiments, the engineered cargo delivery system polynucleotide comprises 1-10, 1-20, 1-30, 1-40, 1-50, 1-100, 1-150, 1-200, 1-250, 1-300, 1-350, 1-400, 1-500, 1-750, 1-1000, 1-1500, 1-2000, 1-2500, 1-3000, 1-3500 or more of the translated and/or untranslated region of the native gamma or beta nodavirus RNA2.

In certain example embodiments, the cargo polynucleotide encodes a protein, an RNA, or both. In certain example embodiments, the cargo encodes an RNA, wherein the RNA is not translated. In certain example embodiments, the cargo polynucleotide encodes an RNA, wherein the RNA is an interfering RNA, a gene silencing oligonucleotide, or a guide RNA for a CRISPR-Cas system.

In certain example embodiments, the cargo is a hairpin RNA, optionally a long hairpin RNA. In certain example embodiments, the one or more regulatory elements operatively coupled to the cargo polynucleotide comprises a Pol III promoter and optionally a Poll III termination sequence.

In certain example embodiments, the cargo polynucleotide encodes an RNA, wherein the RNA is an interfering RNA, a gene silencing oligonucleotide, or a guide RNA for a CRISPR-Cas system, and wherein the gene silencing oligonucleotide, the interfering RNA, and the guide RNA are capable of specifically hybridizing with or targeting a pathogenic DNA or RNA. In certain example embodiments, the pathogenic DNA or RNA is a pathogenic DNA or RNA from a shrimp, a prawn, and/or a fish pathogen. In certain example embodiments, the shrimp, prawn, or fish pathogen is a virus, a bacteria, a fungus, a worm, or a protozoan. In certain example embodiments, the shrimp or prawn pathogen is white spot syndrome virus (WSSV), Yellow head virus (YHV), Gill associated virus (GAV), Infectious hypodermal and hematopoietic necrosis virus (IHHNV), Taura syndrome virus (TSV), Monodon baculovirus (MBV), Hepatopancreatic parvovirus (HPV), Related Australian lymphoid organ virus (LOV), Mourilyan virus (MOV), Laem Singh virus (LSNV), Baculovirus midgut gland necrosis virus (BMNV), Monodon slow growth syndrome (MSGS), Macrobrachium rosenbergii nodavirus (MrNV), Extra small virus (XSV), Spawner-isolated mortality virus (SMV), Lymphoidal parvo-like virus (LPV), Baculovirus penaei (BP), Shrimp iridovirus (SIV), Shrimp hemocyte iridescent virus (SHIV), Reo-like virus (REO) type II and IV, Lymphoid organ vacuolization virus (LOVV), Rhabdovirus of penaeid shrimp (RPS), Infectious myonecrosis virus (IMNV), a nodavirus, a bacteria of the genus Vibrio, a Rickettsiae, a fungs of the genus Langenidium, a fungus of the genus Sirolopidium, a fungus of the genus Fusarium, a protozoan of the genus Micropsoran, a protozoan of the genus Haplospora, a protozoan of the genus Gregarina, a trematode (e.g., of the family Opecoelidae, Microphalliadae, or Echinostomatidae), a cestode (e.g., of the genus Prochristianella, Parachristianella, or Renibulbus), a nematode (e.g., Spirocamallanus pereirai, Leptolaimus sp. Ascaropsis sp, and Hysterothylacium reliquens), Enterocytozoon hepatopenaei (EHP), Infectious myonecrosis virus, or infection with HPR-deleted or HPRO infectious salmon anemia virus, Epizootic Haematopoietic Necrosis (EHN), Infectious Haematopoietic Necrosis (IHN), Infection with koi herpesvirus, Infection with red sea bream iridovirus, Infection with salmonid alphavirus, Oncorhynchus masou Virus (OMV), Infectious Pancreatic Necrosis (IPN), Viral Encephalopathy and Retinopathy (VER), Spring Viraemia of Carp (SVC), Channel Catfish Virus (CCV) Viral Haemorrhagic Septicaemia (VHS), Lymphocystis, Bacterial Kidney Disease (BKD), Epizootic Ulcerative Syndrome (EUS), or Nervous Necrosis Virus (NNV).

In certain example embodiments, the cargo polynucleotide is a gene of interest or portion thereof. In certain example embodiments, the cargo polynucleotide encodes a Cas protein or portion thereof. In some embodiments, the cargo polynucleotide encodes one or more CRISPR-Cas system polynucleotide. Other exemplary cargo molecules are described elsewhere herein.

Described in several exemplary embodiments herein are engineered cargo delivery polynucleotides containing an engineered gamma or beta nodavirus RNA 1 containing a 5′ untranslated region of a native gamma or beta nodavirus; a 3′ untranslated region of a native gamma or beta nodavirus; a cargo polynucleotide; and optionally a complete translated region of a native gamma or beta nodavirus RNA 1 or a portion thereof, wherein the cargo delivery polynucleotide is located between the 5′ untranslated region and the 3′ untranslated region, and wherein the optional complete translated region of a native gamma or beta nodavius RNA 1 or portion thereof is located between the 5′ untranslated region and the 3′ untranslated region of the engineered gamma or beta nodavirus RNA 1.

Exemplary engineered cargo delivery polynucleotides are described and demonstrated elsewhere herein, see e.g., the Working Examples herein.

In some embodiments, the native gamma or beta nodavirus is selected from Macrobrachium rosenbergii nodavirus (MrNV), Penaeus vannamei nodavirus (PvNV), covert mortality nodavirus (CMNV), Farfantepenaeus duorarum nodavirus (FdNV), tiger puffer nervous necrosis virus (TPNNV), striped jack nervous necrosis virus (SJNNV), barfin flounder nervous necrosis virus (BFNNV), red-spotted grouper nervous necrosis virus (RGNNV), or an Egyptian Nile tilapia nervous necrosis virus (Egyptian NT NNV) (see e.g., Taha et al. Aquaculture International (2020) 28:1811-182).

In some embodiments, the engineered polynucleotide does not contain any portion of the translated region of the native gamma nodavirus. In some embodiments, the engineered cargo delivery polynucleotide does not contain any portion of a translated RNA dependent polymerase of a native nodavirus such as a gamma or beta nodavirus.

In some embodiments, at least one or more polynucleotides of the translated region (e.g., open reading frame) of the native gamma or beta nodavirus is removed. In some embodiments, the number of nucleotides removed is the minimal number to render a resulting viral particle replication incompetent. In some embodiments, the engineered polynucleotide includes 1-10, 1-20, 1-30, 1-40, 1-50, 1-100, 1-150, 1-200, 1-250, 1-300, 1-350, 1-400, 1-500, 1-750, 1-1000, 1-1500, 1-2000, 1-2500, 1-3000, 1-3500 consecutive or nonconsecutive polynucleotides or more of the translated region (e.g., open reading frame) and/or untranslated region of the native gamma nodavirus RNA2, or capsid forming domain thereof.

### Cargo Polynucleotides

The cargo delivery polynucleotide (also referred to herein as a cargo “polynucleotide”) can be any desired polynucleotide to be delivered. In some embodiments, the cargo polynucleotide encodes a protein and/or an RNA. The engineered cargo delivery polynucleotide of any one of the preceding claims, wherein the cargo encodes an RNA, wherein the RNA is not translated (i.e., is a non-coding RNA). In some embodiments the non-translated RNA is a hairpin RNA (e.g., a short hairpin RNA (shRNA), long hairpin RNA (lhRNA), and/or the like), a microRNA, long non-coding RNA or other non-translated RNA. In some embodiments, the cargo polynucleotide encodes an RNA, wherein the RNA is an interfering RNA or a guide RNA for a CRISPR-Cas system. In some embodiments, the RNA is a translated RNA (e.g., an mRNA). In some embodiments, the mRNA encodes a Cas polypeptide. In some embodiments, the cargo polynucleotide encodes a translated and an untranslated RNA. In some of these embodiments, the translated polynucleotide is a Cas polypeptide, and the untranslated RNA is one or more guide polynucleotides for a CRISPR-Cas system.

In some embodiments, the cargo polynucleotide encodes an RNA, wherein the RNA is an interfering RNA, a gene silencing oligonucleotide, or a guide RNA for a CRISPR-Cas system, and wherein the gene silencing oligonucleotide, the interfering RNA, and the guide RNA are capable of specifically hybridizing with or targeting a pathogenic DNA or RNA. In some embodiments, the pathogenic DNA or RNA is a pathogenic DNA or RNA from a shrimp, prawn, and/or fish pathogen. In some embodiments, the shrimp, prawn, or fish pathogen is a virus, a bacteria, a fungus, a worm, or a protozoan.

In some embodiments, the shrimp, prawn, or fish pathogen is white spot syndrome virus (WSSV), Yellow head virus (YHV), Gill associated virus (GAV), Infectious hypodermal and hematopoietic necrosis virus (IHHNV), Taura syndrome virus (TSV), Monodon baculovirus (MBV), Hepatopancreatic parvovirus (HPV), Related Australian lymphoid organ virus (LOV), Mourilyan virus (MOV), Laem Singh virus (LSNV), Baculovirus midgut gland necrosis virus (BMNV), Monodon slow growth syndrome (MSGS), Macrobrachium rosenbergii nodavirus (MrNV), Extra small virus (XSV), Spawner-isolated mortality virus (SMV), Lymphoidal parvo-like virus (LPV), Baculovirus penaei (BP), Shrimp iridovirus (SIV), Shrimp hemocyte iridescent virus (SHIV), Reo-like virus (REO) type II and IV, Lymphoid organ vacuolization virus (LOVV), Rhabdovirus of penaeid shrimp (RPS), Infectious myonecrosis virus (IMNV), a nodavirus, a bacteria of the genus Vibrio, a Rickettsiae, a fungs of the genus Langenidium, a fungus of the genus Sirolopidium, a fungus of the genus Fusarium, a protozoan of the genus Micropsoran, a protozoan of the genus Haplospora, a protozoan of the genus Gregarina, a trematode (e.g., of the family Opecoelidae, Microphalliadae, or Echinostomatidae), a cestode (e.g., of the genus Prochristianella, Parachristianella, or Renibulbus), a nematode (e.g., Spirocamallanus pereirai, Leptolaimus sp. Ascaropsis sp, and Hysterothylacium reliquens), Enterocytozoon hepatopenaei (EHP), Infectious myonecrosis virus, or infection with HPR-deleted or HPRO infectious salmon anemia virus (ISAV), Epizootic Haematopoietic Necrosis (EHN), Infectious Haematopoietic Necrosis (IHN), Infection with koi herpesvirus, Infection with red sea bream iridovirus, Infectious Spleen & Kidney Necrosis Virus (ISKNV), Infection with salmonid alphavirus, Oncorhynchus masou Virus (OMV), Infectious Pancreatic Necrosis (IPN), Viral Encephalopathy and Retinopathy (VER), Spring Viraemia of Carp (SVC), Channel Catfish Virus (CCV) Viral Haemorrhagic Septicaemia (VHS), Lymphocystis, Bacterial Kidney Disease (BKD), Epizootic Ulcerative Syndrome (EUS), Nervous Necrosis Virus (NNV), Tilapia Lake Virus (TiLV), or any combination thereof.

In some embodiments, the cargo polynucleotide is a gene of interest or portion thereof. In some embodiments, the cargo polynucleotide encodes a polypeptide of interest. In some embodiments, the cargo polynucleotide encodes a Cas protein or portion thereof and/or one or more guide RNAs for a CRISPR Cas system.

### Vectors and Vector Systems

Also described herein are engineered delivery vectors and vector systems that can include a cargo polynucleotide as described elsewhere herein. In some embodiments, a vector can include a cargo polynucleotide as described elsewhere herein, wherein the cargo polynucleotide is operatively coupled to one or more regulatory elements.

In some embodiments, the vector includes one or more components of a baculoviral vector. Exemplary baculoviral vectors are described elsewhere herein. Exemplary baculoviral are also vectors and/or components thereof are demonstrated in the Working Examples herein. Other suitable vectors will be appreciated by one of ordinary skill in the art.

Also described herein are engineered delivery systems for delivering a cargo nucleic acid including a cargo polynucleotide as described elsewhere herein, wherein the cargo polynucleotide is operatively coupled to one or more regulatory elements; and an engineered gamma or beta nodavirus capsid polynucleotide encoding a native gamma or beta nodavirus capsid polypeptide, wherein the engineered gamma or beta nodavirus capsid polynucleotide is operatively coupled to one or more regulatory elements. In some embodiments, the regulatory element is a promoter. In some embodiments the regulatory element operatively coupled to the cargo is a Pol II promoter. In some embodiments, the regulatory element operatively coupled to the cargo is a Pol III promoter. In some embodiments, the regulatory element operatively coupled to the gamma or beta nodavirus capsid polynucleotide is a Pol II promoter or a Pol III promoter. In some embodiments, where a nodavirus envelope protein encoding polynucleotide is included in the vector the nodavirus envelope protein encoding polynucleotide is operatively coupled to a Pol II or a PolII promoter. In some embodiments, the Pol II promoter is selected from P10 or PpH. In some embodiments, the Pol III promoter is a U6, a 7SK, or an H1 promoter. Other suitable promoters can be used and are described elsewhere herein and will be appreciated by one of ordinary skill in the art.

In some embodiments, the engineered gamma or beta nodavirus capsid polynucleotide comprises a 5′ untranslated region of a native gamma or beta nodavirus RNA 2, a translated region of native a gamma or beta nodavirus RNA 2, and a 3′ untranslated region of a native gamma or beta nodavirus RNA 2. Exemplary gamma and/or beta nodavirus RNA 2 polynucleotides are demonstrated in the Working Examples herein.

In certain example embodiments, the native gamma or beta nodavirus is selected from Macrobrachium rosenbergii nodavirus (MrNV), Penaeus vannamei nodavirus (PvNV), covert mortality nodavirus (CMNV), Farfantepenaeus duorarum nodavirus (FdNV), tiger puffer nervous necrosis virus (TPNNV), striped jack nervous necrosis virus (SJNNV), barfin flounder nervous necrosis virus (BFNNV), red-spotted grouper nervous necrosis virus (RGNNV), or an Egyptian Nile tilapia nervous necrosis virus (Egyptian NT NNV).

In certain example embodiments, the engineered cargo delivery polynucleotide and the engineered polynucleotide encoding a native gamma or beta nodavirus capsid are on the same vector.

In some embodiments, the engineered cargo delivery polynucleotide and the engineered polynucleotide encoding a native gamma or beta nodavirus capsid are on different vectors.

In some embodiments, the vector system is capable of producing virus or viral like particles. In some embodiments, the virus or viral like particles contain the cargo polynucleotide.

In some embodiments, the regulatory element operatively coupled to the cargo is P10, PpH, U6, 7SK, or H1.

In some embodiments, the vector system is capable of packaging the cargo delivery polynucleotide into the virus or viral like particles.

In some embodiments, the vector system and/or virus or viral like particles produced therefrom is/are replication incompetent.

In some embodiments, the engineered an engineered cargo delivery polynucleotide and/or the engineered gamma or beta nodavirus capsid polynucleotide is included in a baculoviral vector.

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6, 7SK, and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the R-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit 0-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, O-actin, RSV, and PGK, optionally a shrimp or fish beta-actin or shrimp or fish EF-1α, or a white spot syndrome virus promoter. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Ferl14), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Other regulatory elements can be included in the vectors or constructs therein. In some embodiments, the vector or one or more of the constructs therein include a Pol II or Pol III transcription termination sequence. Such termination sequences can be incorporated at the end of e.g., a cargo polynucleotide to be expressed and/or after a nodavirus structural protein (e.g., a capsid and/or envelope protein) encoding polynucleotide. Exemplary Pol II termination sequences include, but are not limited to, poly A signal (e.g., SV40 polyA signal). Exemplary Pol III termination sequences include T, TT, TTT, TTTT, TTTTT, TTTTTT, TTTTTTT, TTTTTTTT, and others. See e.g., Gao et al 2018. Mol. Ther. 10:36-44.

### Selectable Markers and Tags

One or more of the engineered polynucleotides can be operably/operatively linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered polynucleotide such that the selectable marker polypeptide, when translated, is inserted between the C and/or N terminus of a cargo polypeptide or inserted at either end of a cargo polypeptide. In some embodiments, the selectable marker or tag is a polynucleotide barcode or unique molecular identifier (UMI).

It will be appreciated that the polynucleotide encoding such selectable markers or tags can be incorporated into an engineered polynucleotide encoding one or more components of the nodavirus delivery system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

### Codon Optimization of Vector Polynucleotides

In some embodiments, the engineered polynucleotides and/or vectors can be codon optimized for expression in a particular host cell, such as bacterial, insect, or crustacean. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gown, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59. Such codon optimized sequences are within the ambit of the ordinary skilled artisan in view of the description herein.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

### Cell-Based Vector Amplification and Expression

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for expression of one or more elements of the engineered nodaviral delivery system described herein (e.g. nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells.

In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U20S, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2p plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

### Cells and Virus Like Particles (VLPS)

Described in several embodiments herein are cells that include an engineered cargo delivery polynucleotide, an engineered delivery vector, an engineered delivery vector system, and/or formulation as described in greater detail elsewhere herein. In some embodiments, the cell is a bacterial cell, a mammalian cell, a crustacean cell, a fish cell, a yeast cell, or an insect cell. In some embodiments, the cell is a bacterial or an insect cell. In some embodiments, the cell is non-pathogenic to shrimp, prawns, or other crustaceans. In some embodiments, the cell is capable of producing virus like particles comprising the cargo polynucleotide. In some embodiments, the cell is capable of expressing the vector or vector system described elsewhere herein.

Described in several exemplary embodiments herein engineered gamma or beta nodavirus or nodavirus like particles that can be produced by a cell described herein and that can contain a cargo polynucleotide. The engineered gamma or beta nodavirus or nodavirus like particles can be generated by a method that includes expressing a vector or vector system as described elsewhere herein in a cell capable of expressing the vector or vector system.

Described in certain example embodiments herein are engineered gamma or beta nodavirus or nodavirus like particles produced from and/or containing an engineered cargo delivery system polynucleotide of the present disclosure described herein and/or engineered cargo delivery vector of or vector system thereof.

Described in certain example embodiments herein is a cell or population thereof comprising an engineered cargo delivery system polynucleotide of the present disclosure described herein, an engineered cargo delivery vector or vector system comprising the engineered cargo delivery system polynucleotide, an engineered nodavirus or nodavirus like particle or population thereof produced from the engineered cargo delivery system polynucleotide, and/or a formulation thereof.

In certain example embodiments, the cell or population thereof is a bacterial cell or population thereof or an insect cell or population thereof. In certain example embodiments, the cell or population thereof is non-pathogenic to shrimp, prawns, or other crustaceans, or fish, optionally a fin fish. In certain example embodiments, the cell or population thereof is capable of producing the engineered nodavirus or nodavirus like particles comprising the cargo polynucleotide.

### Virus Particle and Virus Like Particle (VLP) Production

Described in certain example embodiments are methods of producing engineered gamma or beta nodavirus or nodavirus like particles carrying a cargo expressing an engineered cargo delivery system polynucleotide of the present disclosure described herein, an engineered cargo delivery vector or vector system comprising the engineered cargo delivery system polynucleotide in a cell capable of expressing the engineered cargo delivery system polynucleotide, vector, vector system, or any combination thereof.

Described in certain example embodiments is an engineered gamma or beta nodavirus or nodavirus like particle or population thereof produced by the method of the present disclosure described herein.

In some embodiments, one or more engineered nodaviral vectors and/or system thereof can be delivered to a suitable cell line for production of virus particles or virus like particles containing the cargo polynucleotide to be delivered to a host cell. In some embodiments, the virus particles or virus like particles are replication incompetent. Suitable cells include bacterial and insect cells (e.g., Sf9 cells). In some embodiments, after delivery of one or more viral vectors to the suitable host cells for or virus production from viral vectors and systems thereof, the cells are incubated for an appropriate length of time to allow for viral gene expression from the vectors, packaging of the cargo polynucleotide, virus particle assembly, and secretion of mature virus particles into the culture media. Various other methods and techniques are generally known to those of ordinary skill in the art.

In some embodiments mature virus particles and/or VLPs can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ particles/mL.

### Formulations

Described herein are formulations containing an engineered cargo delivery polynucleotide, an engineered delivery vector, and/or an engineered delivery vector system as described in greater detail elsewhere herein. In some embodiments, the formulation is a pharmaceutical formulation, where the formulation further includes a pharmaceutically acceptable carrier. In some embodiments, as described elsewhere herein, the formulation can be administered to a subject, such as prawn, shrimp, or other crustacean, or fish, optionally a fin fish. In some embodiments, delivery is to the subject via injection or oral dosing or via ingestion or absorption from the environment external to the subject such as via a or a feed formulation or water source. Other suitable administration routes are described elsewhere herein and will be appreciated by those of ordinary skill in the art in view of the description herein.

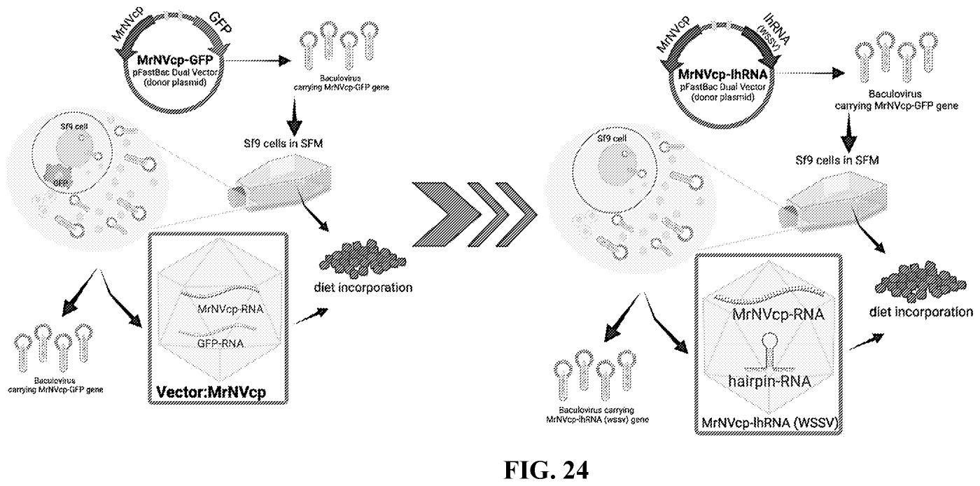

Described herein are feed formulations that can contain cells that contain and/or capable of producing the engineered nodavirus (e.g., gamma or beta nodavirus) or virus like particles described herein. In some embodiments the feed formulations contain the engineered the engineered nodavirus (e.g., gamma or beta nodavirus) or virus like particles described herein. In some embodiments, the engineered nodavirus (e.g., gamma or beta nodavirus) or virus like particles described herein are produced in a suitable in vitro or bioreactor system, harvested or otherwise purified from the system, and added to a feed formulation. In some embodiments, the feed formulation is formulated for consumption by a shrimp, prawn, or other crustacean, or fish, particularly a fin fish. When consumed by a subject, such as prawn, shrimp, or other crustacean, or fish, particularly a fin fish the virus particles or VLPs and/or cells containing and/or capable of producing the virus particles and/or virus like particles are ingested by the subject, thus delivering the cargo delivery polynucleotide to the subject. The engineered gamma or beta nodavirus particles or virus like particles can be capable of transducing and delivering their polynucleotide cargo to cells that are infected by the native gamma or beta nodavirus is from. See also e.g., FIG. 24.

In some in embodiments, the formulation containing the is a feed formulation adapted for feeding a non-human animal. In some embodiments, the animal is a prawn, shrimp, or other crustacean, or fish, particularly a fin fish. In some embodiments, the formulation is a feed formulation that is formulated as a complete diet. In some embodiments, the formulation is a feed supplement. In some embodiments, the formulation is a feed additive. As used herein, “complete diet” refers to a feed formulation that contains all the nutrients, calories, minerals, vitamins, and other components needed to meet the dietary requirements of an animal without any additional feed sources and intended to be fed as the entire food source fir the animal. As used herein “feed additive” is an extra nutrient or non-nutrient component that is provided or can be added to a feed or diet that is beyond or in addition to the basic nutritional components of a feed or diet. Feed additives generally fall or can include components that fall into five basic categories: technological additives (e.g., preservatives, antioxidants, emulsifiers, stabilizing agents, acidity regulators, binding agents, silage additives, and/or the like), sensory additives (e.g., flavors, colorants, and/or the like), nutritional additives (e.g., vitamins, minerals, amino acids, trace elements and other minerals, and/or the like), zootechnical additives (e.g., digestibility enhancers, enzymes, intestinal health enhancers, microbes, fiber, and/or the like), and pharmaceuticals. Technological additives are substances or compositions that serves a technological purpose in a feed formulation. Sensory additives are substances or compositions that improve or otherwise changes the organoleptic properties of the feed, or the visual characteristics of the food or other product derived from animals. Nutritional additives are any nutrient substance or component. Zootehcnical additives are substances and compositions that improve, favorably affect, or otherwise modify the health and/or performance of an animal and/or modify their impact on the environment. It will be appreciated that any one particular feed additive or component can fall into more than one category. Feed additives can be added to a feed formulation at any stage during feed formulation production (such as being provided in or as a pre-mix) or provided to an animal as a feed supplement that is separate from the feed formulation and added at the point of feeding. As used herein, “feed supplement” refers to a composition formulated for consumption by an animal and intended to be fed undiluted as a supplement to other feeds or offered free choice with other parts of the ration separately available or further diluted and mixed to produce a complete feed ration. A feed additive are intended to be fully incorporated into a feed formulation, while feed supplements indented to be stand-alone compositions that can be fed free choice or mixed in with a feed ration at point of feeding or can be mixed into a feed formulation to make a complete feed formulation. A feed supplement can include one or more feed additive(s).

In some embodiments, the feed formulation is formulated to meet the nutritional requirements of a specific age or life-stage or provide some other benefit that is specific to age or life stage. In some embodiments, the feed formulation is formulated to support or meet specific requirements of an animal in a diseased or otherwise non-healthy or normal state. In some embodiments, the feed formulation is formulated to enhance or support the performance of the animal to which it is fed.

### Kits

Any of the engineered polynucleotides, vectors, vector systems, compositions, formulations, particles (e.g., virus particles, VLPs), or cells described herein, or a combination thereof can be presented as a combination kit. As used herein, the terms “combination kit” or “kit of parts” refers to the engineered polynucleotides, vectors, vector systems, compositions, formulations, particles (e.g., virus particles, VLPs), or cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the engineered polynucleotides, vectors, vector systems, compositions, formulations, particles (e.g., virus particles, VLPs), or cells described herein or a combination thereof to a subject in need thereof. In some embodiments, the subject in need thereof is a crustacean such as a shrimp or prawn. In some embodiments, the subject is in need of treatment or prevention of infection by a pathogen, wherein the pathogen is white spot syndrome virus (WSSV), Yellow head virus (YHV), Gill associated virus (GAV), Infectious hypodermal and hematopoietic necrosis virus (IHHNV), Taura syndrome virus (TSV), Monodon baculovirus (MBV), Hepatopancreatic parvovirus (HPV), Related Australian lymphoid organ virus (LOV), Mourilyan virus (MOV), Laem Singh virus (LSNV), Baculovirus midgut gland necrosis virus (BMNV), Monodon slow growth syndrome (MSGS), Macrobrachium rosenbergii nodavirus (MrNV), Extra small virus (XSV), Spawner-isolated mortality virus (SMV), Lymphoidal parvo-like virus (LPV), Baculovirus penaei (BP), Shrimp iridovirus (SIV), Shrimp hemocyte iridescent virus (SHIV), Reo-like virus (REO) type II and IV, Lymphoid organ vacuolization virus (LOVV), Rhabdovirus of penaeid shrimp (RPS), Infectious myonecrosis virus (IMNV), a nodavirus, a bacteria of the genus Vibrio, a Rickettsiae, a fungs of the genus Langenidium, a fungus of the genus Sirolopidium, a fungus of the genus Fusarium, a protozoan of the genus Micropsoran, a protozoan of the genus Haplospora, a protozoan of the genus Gregarina, a trematode (e.g., of the family Opecoelidae, Microphalliadae, or Echinostomatidae), a cestode (e.g., of the genus Prochristianella, Parachristianella, or Renibulbus), a nematode (e.g., Spirocamallanus pereirai, Leptolaimus sp. Ascaropsis sp, and Hysterothylacium reliquens), Enterocytozoon hepatopenaei (EHP), Infectious myonecrosis virus, or infection with HPR-deleted or HPRO infectious salmon anemia virus (ISAV), Epizootic Haematopoietic Necrosis (EHN), Infectious Haematopoietic Necrosis (IHN), Infection with koi herpesvirus, Infection with red sea bream iridovirus, Infectious Spleen & Kidney Necrosis Virus (ISKNV), Infection with salmonid alphavirus, Oncorhynchus masou Virus (OMV), Infectious Pancreatic Necrosis (IPN), Viral Encephalopathy and Retinopathy (VER), Spring Viraemia of Carp (SVC), Channel Catfish Virus (CCV) Viral Haemorrhagic Septicaemia (VHS), Lymphocystis, Bacterial Kidney Disease (BKD), Epizootic Ulcerative Syndrome (EUS), Nervous Necrosis Virus (NNV), Tilapia Lake Virus (TiLV), or any combination thereof.

### Methods of Use

The engineered polynucleotides, vectors, and vector systems, cells, virus particles, and virus like particles described herein can be used to deliver a cargo delivery polynucleotide to a recipient cell.

Described in certain example embodiments are methods of delivering a cargo to a recipient cell or cell population, the method comprising delivering an engineered cargo delivery system polynucleotide of the present disclosure described herein to the recipient cell or cell population, a vector or vector system comprising the engineered cargo delivery system polynucleotide, and/or an engineered beta or gamma nodavirus or virus like particle produced by or containing the engineered cargo delivery system polynucleotide to a subject comprising the recipient cell or cell population and/or to the recipient cell of cell population.

In certain example embodiments, the engineered cargo delivery system polynucleotide of the present disclosure described herein to the recipient cell or cell population, a vector or vector system comprising the engineered cargo delivery system polynucleotide, and/or an engineered beta or gamma nodavirus or virus like particle produced by or containing the engineered cargo delivery system polynucleotide is contained in a formulation. In certain example embodiments, the formulation is a feed formulation, optionally formulated for shrimp, prawn or other crustacean or a fish, optionally a fin fish. In certain example embodiments, the subject is a shrimp, prawn, other crustacean, or fish, optionally a fin fish.

Described in certain example embodiments herein are methods of treating and/or preventing a disease in a crustacean or a fish, optionally a fin fish, comprising delivering to the crustacean or fish or a cell thereof an engineered cargo delivery system polynucleotide of the present disclosure described herein to the recipient cell or cell population, a vector or vector system comprising the engineered cargo delivery system polynucleotide, and/or an engineered beta or gamma nodavirus or virus like particle produced by or containing the engineered cargo delivery system polynucleotide.

In certain example embodiments, the engineered cargo delivery system polynucleotide of the present disclosure described herein to the recipient cell or cell population, a vector or vector system comprising the engineered cargo delivery system polynucleotide, and/or an engineered beta or gamma nodavirus or virus like particle produced by or containing the engineered cargo delivery system polynucleotide is contained in a formulation. In certain example embodiments, the formulation is a feed formulation, optionally formulated for shrimp, prawn or other crustacean or a fish, optionally a fin fish.

In certain example embodiments, the disease is caused by a pathogen, wherein the pathogen is white spot syndrome virus (WSSV), Yellow head virus (YHV), Gill associated virus (GAV), Infectious hypodermal and hematopoietic necrosis virus (IHHNV), Taura syndrome virus (TSV), Monodon baculovirus (MBV), Hepatopancreatic parvovirus (HPV), Related Australian lymphoid organ virus (LOV), Mourilyan virus (MOV), Laem Singh virus (LSNV), Baculovirus midgut gland necrosis virus (BMNV), Monodon slow growth syndrome (MSGS), Macrobrachium rosenbergii nodavirus (MrNV), Extra small virus (XSV), Spawner-isolated mortality virus (SMV), Lymphoidal parvo-like virus (LPV), Baculovirus penaei (BP), Shrimp iridovirus (SIV), Shrimp hemocyte iridescent virus (SHIV), Reo-like virus (REO) type II and IV, Lymphoid organ vacuolization virus (LOVV), Rhabdovirus of penaeid shrimp (RPS), Infectious myonecrosis virus (IMNV), a nodavirus, a bacteria of the genus Vibrio, a Rickettsiae, a fungs of the genus Langenidium, a fungus of the genus Sirolopidium, a fungus of the genus Fusarium, a protozoan of the genus Micropsoran, a protozoan of the genus Haplospora, a protozoan of the genus Gregarina, a trematode (e.g., of the family Opecoelidae, Microphalliadae, or Echinostomatidae), a cestode (e.g., of the genus Prochristianella, Parachristianella, or Renibulbus), a nematode (e.g., Spirocamallanus pereirai, Leptolaimus sp. Ascaropsis sp, and Hysterothylacium reliquens), Enterocytozoon hepatopenaei (EHP), Infectious myonecrosis virus, or infection with HPR-deleted or HPRO infectious salmon anemia virus (ISAV), Epizootic Haematopoietic Necrosis (EHN), Infectious Haematopoietic Necrosis (IHN), Infection with koi herpesvirus, Infection with red sea bream iridovirus, Infectious Spleen & Kidney Necrosis Virus (ISKNV), Infection with salmonid alphavirus, Oncorhynchus masou Virus (OMV), Infectious Pancreatic Necrosis (IPN), Viral Encephalopathy and Retinopathy (VER), Spring Viraemia of Carp (SVC), Channel Catfish Virus (CCV) Viral Haemorrhagic Septicaemia (VHS), Lymphocystis, Bacterial Kidney Disease (BKD), Epizootic Ulcerative Syndrome (EUS), Nervous Necrosis Virus (NNV), Tilapia Lake Virus (TiLV), or any combination thereof.

Described in certain example embodiments herein are kits comprising an engineered cargo delivery polynucleotide of the present disclosure described herein, an engineered cargo delivery vector or vector system of the present disclosure described herein, a cell or population thereof of the present disclosure described herein, an engineered beta or gamma nodavirus or virus like particle of the present disclosure described herein, a formulation of the present disclosure described herein, or any combination thereof.

Described in certain example embodiments are methods of delivering a cargo delivery polynucleotide to a recipient cell or cell population that include delivering a cell, a virus like particle, or a feed source as in any one of claims, to a subject that includes the recipient cell or cell population and/or to the recipient cell of cell population. In some embodiments, the subject is a shrimp, prawn, or other crustacean, or a fish, particularly a fin fish. In some embodiments, particularly those utilizing a feed source, delivery can be oral when the feed source is ingested.

Described in certain exemplary embodiments herein are methods of treating and/or preventing a disease in a crustacean or a fish, that includes delivering to the crustacean or fish or a cell thereof a cell, a virus or virus like particle, or a feed source to the crustacean, fish, or cell thereof. In some embodiments the disease is caused by a virus, a bacteria, a fungus, a worm, or a protozoan. In some embodiments, the disease is caused by a pathogen, wherein the pathogen is white spot syndrome virus (WSSV), Yellow head virus (YHV), Gill associated virus (GAV), Infectious hypodermal and hematopoietic necrosis virus (IHHNV), Taura syndrome virus (TSV), Monodon baculovirus (MBV), Hepatopancreatic parvovirus (HPV), Related Australian lymphoid organ virus (LOV), Mourilyan virus (MOV), Laem Singh virus (LSNV), Baculovirus midgut gland necrosis virus (BMNV), Monodon slow growth syndrome (MSGS), Macrobrachium rosenbergii nodavirus (MrNV), Extra small virus (XSV), Spawner-isolated mortality virus (SMV), Lymphoidal parvo-like virus (LPV), Baculovirus penaei (BP), Shrimp iridovirus (SIV), Shrimp hemocyte iridescent virus (SHIV), Reo-like virus (REO) type II and IV, Lymphoid organ vacuolization virus (LOVV), Rhabdovirus of penaeid shrimp (RPS), Infectious myonecrosis virus (IMNV), a nodavirus, a bacteria of the genus Vibrio, a Rickettsiae, a fungs of the genus Langenidium, a fungus of the genus Sirolopidium, a fungus of the genus Fusarium, a protozoan of the genus Micropsoran, a protozoan of the genus Haplospora, a protozoan of the genus Gregarina, a trematode (e.g., of the family Opecoelidae, Microphalliadae, or Echinostomatidae), a cestode (e.g., of the genus Prochristianella, Parachristianella, or Renibulbus), a nematode (e.g., Spirocamallanus pereirai, Leptolaimus sp. Ascaropsis sp, and Hysterothylacium reliquens), Enterocytozoon hepatopenaei (EHP), Infectious myonecrosis virus, or infection with HPR-deleted or HPRO infectious salmon anemia virus (ISAV), Epizootic Haematopoietic Necrosis (EHN), Infectious Haematopoietic Necrosis (IHN), Infection with koi herpesvirus, Infection with red sea bream iridovirus, Infectious Spleen & Kidney Necrosis Virus (ISKNV), Infection with salmonid alphavirus, Oncorhynchus masou Virus (OMV), Infectious Pancreatic Necrosis (IPN), Viral Encephalopathy and Retinopathy (VER), Spring Viraemia of Carp (SVC), Channel Catfish Virus (CCV) Viral Haemorrhagic Septicaemia (VHS), Lymphocystis, Bacterial Kidney Disease (BKD), Epizootic Ulcerative Syndrome (EUS), Nervous Necrosis Virus (NNV), Tilapia Lake Virus (TiLV), or any combination thereof.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

Viral diseases are a major threat to crustacean aquaculture industry in the US and worldwide. Periodic outbreaks of several major viral diseases have caused catastrophic losses to farmers around the globe and threaten the long-term sustainability of the industry. Among all the viral diseases reported in shrimp and crustacean aquaculture, White Spot Disease (WSD) remains economically the most important disease globally. White Spot Disease caused by the white spot syndrome virus (WSSV) is an OIE-reportable disease that has caused enormous economic losses to shrimp and crayfish aquaculture in the past and is a continuing threat. Therefore, availability of a therapeutic to control WSD and other pathogens, that can be delivered orally can potentially revolutionize crustacean aquaculture worldwide [1,2].

The US imports 1.2 billion pounds of shrimp but only produces 4 million pounds [3]. Most imports of commodity shrimp come primarily from two countries, India and Ecuador. In the US, most of the shrimp farms are in Texas with other farms located in Alabama, Florida, Hawaii, Nevada, Michigan, Indiana, North Carolina, Iowa, Massachusetts and Guam. The overall sustainability and environmental impact of these US farming operations is quite favorable [3] and, with better technologies, they could have a significantly greater positive impact on both coastal and inland communities.

The US farm-raised shrimp production is based on production of specific pathogen free (SPF) Pacific white shrimp (Penaeus vannamei) seed in a small number of hatcheries. Reliance on SPF shrimp does not guarantee that disease outbreak will not occur in the farm nor does it provide resistance to significant diseases including WSD. In the US, shrimp farms rely on biosecurity and containment to reduce disease. Such farms have a post-larval (PL) growing area where the density of shrimp cultured is about 1500 per m³ as the starting area from the hatchery. They have juveniles stocked at ˜400 shrimp per m³ and grow-out raceways stocked at ˜200 shrimp per m³. While these are open ponds, they are often under greenhouses to reduce the potential for contamination by aerosols or waterfowl. Nonetheless, pathogen contamination poses a significant risk factor as the systems are used over the long term. Additionally, the use of high stocking density and water recycling strategies makes the farms vulnerable to system wide epizootics that could be economically devastating. The trend toward high intensity (high stocking density) systems will demand better surveillance methods, more rapid response and effective methods to stop and cure a viral outbreak. This cannot be done through management and disease free broodstock and seed alone.

White spot disease caused by WSSV is the economically most important disease in shrimp and other crustacean aquaculture. The disease was first reported in East Asia during 1992 to 1993 [4] and rapidly spread throughout shrimp farming regions of Southeast Asia and North America in the mid-1990s [5,6]. During 1999, WSD severely impacted the shrimp industries of both Central and South America. WSD is now present in most shrimp farming countries in Asia, the Middle East, North, South and Central America and Australia [1,2,6,7, 11]. It has been estimated that the losses due to WSD since its emergence are $6 billion in Asia and $1-2 billion in the Americas [2]. WSD outbreaks in Mozambique in 2011, Madagascar in 2012, and Australia in 2016 had a devastating consequence on shrimp industry in these countries.

While WSD makes a lot of news in other countries where shrimp aquaculture is extensive, it also impacts US crustacean aquaculture. The most recent example is the outbreak in red crawfish in Louisiana in 2017 [8]. The disease has already been detected in ponds in LA in 2019 [9], so WSD is a current threat to the $350 million-dollar US crayfish aquaculture industry. WSD had been shown to be present in Louisiana crawfish farms in the past [10], but the recent wild swings in temperature may have made crawfish more susceptible to WSD in the past few years.

The crayfish industry in the US is mostly centered in Louisiana where 90-95% of the US harvest occurs. In Louisiana, there are 1,600 farmers that manage 111,000 acres of ponds and 800 commercial fishermen associated with the crawfish industry that generates 120-150 million pounds of crawfish annually generating $300 million in associated sales. Recent outbreaks of WSD in this industry are a powerful threat.

In 2012, there was an outbreak of WSSV in Hawaii that killed all the shrimp in the Sunrise Capital shrimp farm in Kekaha [12]. To show the importance attached to viral disease control, the State of Hawaii planned to invest over one million dollars to ensure that the shrimp hatchery can be made free of disease. Unless completely enclosed and under strong biosecurity, shrimp farms are threatened by introduction of viral diseases from seabirds as well as other crustaceans.

There are significant challenges in developing therapies against WSD and other viral diseases in crustaceans: This is because: 1) There is no immortal cell line in shrimp or for any other crustacean. As a result, primary cell cultures derived from hemocytes, hematopoietic tissues, and ovaries of shrimp used to study shrimp viruses [39]. However, conducting basic and applied research using a homogenous population of primary cells can be a difficult and arduous process, 2) Shrimp do not have antibody-based immunity, thus conventional vaccines are not applicable. 3) Due to the lack of antibody-based immunity, recombinant proteins need to be delivered continuously or their protection against disease is lost when they are cleared by the animal, 4) Oral delivery of the recombinant protein or therapeutic RNA molecules need to be accomplished for commercial viability and applicability to crustacean aquaculture. 5) The development of a successful oral delivery of an anti-viral molecule in crustaceans requires that the protein/nucleic acid molecules remain stable in an aqueous milieu and in the gut but be effectively absorbed by the intestinal epithelial layer in the gut while remaining active. 6) Stability of recombinant molecules (protein/nucleic acid) is critically important since these molecules need to be mixed with shrimp feed. In addition, since crustacean farming is practiced primarily in areas where the temperature and humidity are high, feed containing recombinant molecules (protein/RNA) needs to be stable under these harsh environmental conditions. Until the challenges are addressed adequately, the development of an anti-viral therapy in shrimp and crayfish for farm application will remain elusive.

White Spot Syndrome Virus (WSSV): WSSV is a double-stranded circular DNA (dsDNA) containing virus [13]. The WSSV virion consists of a nucleocapsid, tegument and envelope, and it includes at least 58 structural proteins [14-16]. The virions are enveloped, cylindrical to elliptical in shape with a tail like appendage at one end of the particle. The virions are 80-120 nm in width 250-380 nm in length. WSSV is extremely virulent, has a wide host range and targets various tissues [17-19]. The size of the WSSV genome is approximately 300 kbp (297-307 kb in size, and fourteen WSSV isolates have been sequenced:—China, WSSV-CN-1, CN-2, CN-3, CN-4, CN-5; Thailand, WSSV-TH; Taiwan WSSV-TW; Ecuador, WSSV-EC; India, WSSV-I; Brazil, WSSV-Br; Australia, WSSV-Au; Mexico, WSSV-Mex; Egypt, WSSV-EG; and South Korea, WSSV-SK). The viral genome contains 531 putative open reading frames (ORFs), and most of the predicted proteins (73%) show no significant similarity to proteins from other viruses. Based on its biological properties and unique genomic features, WSSV has been classified into anew family Nimaviridae, genus Whispovirus[20].

WSSV encodes a large number of structural proteins (total 40), and major structural proteins include VP664, VP28, VP26, VP24, VP19 and VP15. The viral proteins have been categorized into three groups—envelope proteins (e.g., VP28 and VP19), tegument proteins (e.g., VP26 and VP24) and nucleocapsid proteins (e.g., VP664 and VP15). Among the structural proteins, VP28 is the most abundant envelope protein and polyclonal antibodies against VP28 protein were shown to neutralize WSSV infectivity [21, 22]. A small shrimp GTP-binding membrane protein, Rab7, has been identified from hemocyte membranes that interacts with VP28 and serves as a receptor for WSSV binding to the cell [23]. The envelope protein VP28 and tegument protein VP26 have been the major targets for developing anti-WSSV therapies in shrimp.

Control of WSD using RNA interference (RNAi):_Since the emergence of WSD in the early 1990's and for the colossal losses it caused to shrimp farmers around the globe, an intense effort was made to develop management strategies to contain WSD. Major progress has been made in preventing its spread through biosecurity and development of conventional and molecular diagnostic tools. However, there is no approved and effective therapeutic for the treatment of WSD. Due to the nature of the crustacean immune system (i.e., lack of an adaptive immune system), effective treatments must rely on innate immunity or passive immunity (i.e., introduction of exogenous antibodies). Previous efforts focused mostly on stimulation of the innate immune response or attacking the virus directly through alternative strategies, such as immunostimulants, recombinant proteins, RNAi molecules. Among these approaches, RNAi-based therapy has shown promise although there is no commercially available therapeutic due to the lack of an effective delivery method of RNAi molecules via oral route.

RNA interference has been identified as a mechanism through which shrimp can achieve antiviral immunity. The silencing of a cognate gene by RNAi is based on a conserved nucleic-acid mechanism among fungi, plants and animals that involves in endogenous gene expression regulation and antiviral mechanism [24]. The RNAi mechanism is initiated by a dsRNA, either endogenous or exogenous via cell uptake. The dsRNA is digested by the Dicer molecule into small effector RNAs such as small interfering RNAs (siRNAs). Generally, siRNAs can be formed from repetitive sequences, foreign dsRNA (i.e., viral), or long hairpin-forming transcripts. The siRNA duplex is unwounded, where one strand forms complex with an Argonaute (Ago) protein and serves as the guide RNA of the formed RNA-induced silencing complex (RISC). The guide RNA directs the RISC to seek the mRNA target matching its sequence causing mRNA degradation or repressing protein translation. In shrimp, components of the RNAi machinery Dicer and Ago genes have been identified in Penaeus vannamei, P. monodon and Kuruma prawn Marsupenaeus japonicus [25-28]. Moreover, the genes Dicer2 and Ago2 in M. japonius were proven to be essential in the biogenesis of siRNA and anti-viral mechanism in shrimp [29]. RNAi has been used to target genes from viruses causing severe infections in shrimp such as yellow head virus (YHV), and Taura syndrome virus (TSV) and WSSV [30-33].

The sequence-specific dsRNA targeting viral genes proved to be successful in interfering with viral replication in a dose-dependent manner [32]. However, identifying the correct target genes essential for virus infectivity is crucial in the effectivity of RNAi. For WSSV, essential genes that have been targeted for RNAi include immediate-early genes (ie1-3); early transcribed DNA polymerase (dnapol) and ribonuclease reductase small subunit (rr1&2); and major late structural proteins VP26 and VP28 [34]. Comparative studies were performed to determine the gene target and target combinations that give better protection against WSSV. Among the essential genes, targeting VP28 and rr1 gave more protection (90% and 93.3% survival, respectively) as compared to dnapol and VP281 [35]. Targeting of non-structural gene such as VP9/ICP11 was also found to be effective in improving immunity among different shrimp species M. japonicus, P. monodon, and freshwater prawn Machrobrachium rosenbergii [36].

Delivery of dsRNA by intramuscular injection (IM) remains the most frequently used mode of dsRNA delivery in shrimp studies involving gene knock-down of host and viral genes. This mode, however effective, is only feasible in a small-scale setting performed in the laboratory with proper equipment and resources to produce enough quantity of dsRNA [37]. Application of RNAi to a farm scale is only possible with a cost-effective large-scale production of dsRNA and delivery via oral route. A formaldehyde-inactivated E. coli expressing WSSV dsRNA-VP28 top coated feeds showed promising results in controlling WSD although it provided lower protection compared to IM injection of the same construct [38]. The lower protection was possibly due to the loss of dsRNA during the course of oral delivery. To overcome this, there is a need to develop an oral delivery method that enables RNAi molecules to pass through the gut without getting degraded, get absorbed in the gut epithelial cells and hemocytes, and remain effective in protecting against the corresponding viral disease.

This Example demonstrates engineering of viral vector based on the virus, M. rosenbergii nodavirus (MrNV), which is known to infect shrimp through oral route. In addition, MrNV infects gill tissue, head muscle, heart, abdominal muscle, ovaries, pleopods and tail muscle, just like WSSV. Therefore, a MrNV-based vector can withstand the shrimp gut environment yet deliver the RNA payload in gut epithelial cells and systemically spread to other tissues/cells via hemolymph (hemocytes).

Engineering Infectious cDNA Clone of MrNV and XSV in Insect Cell

As previously discussed, lack of an immortal cell line in crustaceans is a bottleneck in molecular studies involving shrimp viral pathogens. To overcome this limitation, this Example describes the use of recombinant baculovirus to engineer an infectious cDNA clone of freshwater prawn, Macrobrachium rosenbergii nodavirus (MrNV). MrNV is a non-enveloped RNA virus measuring 27 nm in diameter that belongs to the family Nodaviridae. The viral genome consists of two positive sense, single-stranded RNA fragments, RNA-1 (3202 bp) that encodes an RNA-dependent RNA polymerase (RdRp) and a B2 protein, whereas RNA-2 (1175 bp) encodes the viral capsid protein [40]. The post-larvae (PL) of M. rosenbergii are often affected by white tail disease (WTD) caused by MrNV with mortalities reaching as high as 100% in some hatcheries. In WTD, MrNV is usually accompanied by another virus, extra small virus (XSV). XSV is also a non-enveloped, icosahedral virus that is 15 nm in diameter and contains a 796 bp positive-sense, single-stranded RNA genome that encodes a single capsid protein [40].

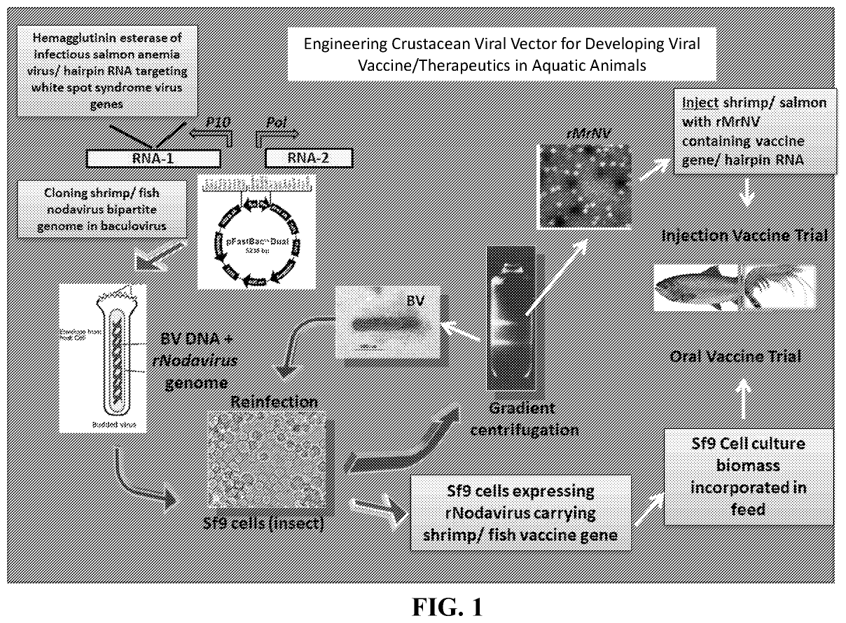

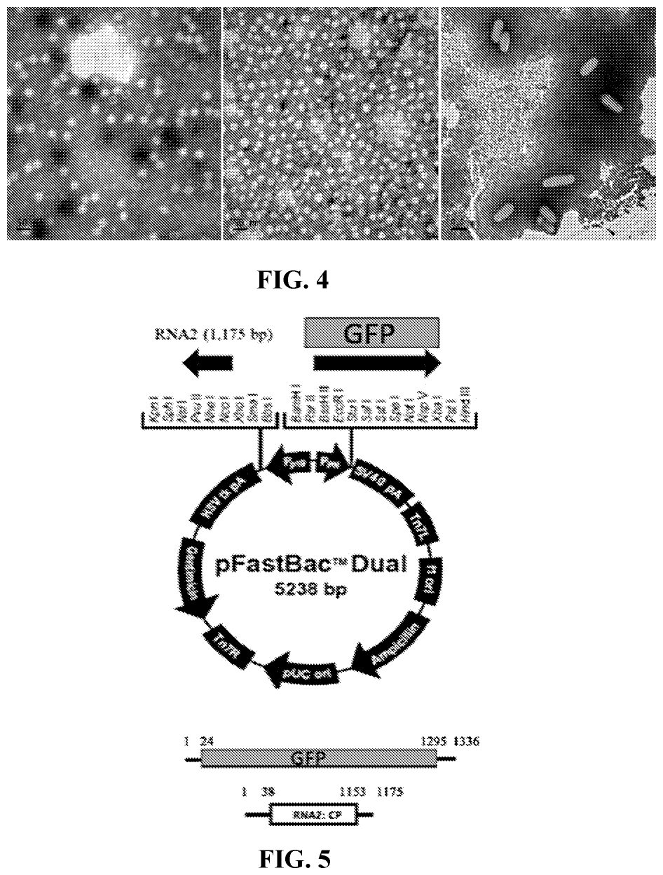

Full-length cDNAs representing MrNV RNA1 and RNA2 were cloned downstream of polyhedrin and P10 promoters in a baculovirus pFastBacDUAL vector. Upon infection of Sf9 cells using recombinant baculovirus (rBV) carrying MrNV RNA1 and RNA2, both rBV and MrNV were produced, and the two viruses were purified from Sf9 cell homogenate by density gradient centrifugation (FIG. 4; also see FIG. 1 for the overall strategy). Immersion-challenge of SPF M. rosenbergii post-larvae (PL) using Sf9 cell derived recombinant MrNV resulted clinical signs in the challenged animals similar to wild type MrNV. Histopathology and RT-PCR assay further confirmed the infectivity of Sf9 cells-derived engineered MrNV.

In freshwater prawn farm, MrNV infection is often associated with a satellite virus, extra small virus (XSV) that does not have a RdRp gene but contains a capsid protein gene. Like the engineered MrNV, XSV was independently cloned in pFastBacDUAL vector and expressed in Sf9 cells. When Sf9 cells-derived MrNV and XSV were used together to challenge SPF M. rosenbergii, the severity of infection and mortality was higher than MrNV alone [41].

The concept of using rBV to produce infectious clones of a shrimp RNA virus was first demonstrated using Taura syndrome virus (TSV) of shrimp by the PI [42]. The ability to engineer a RNA virus of shrimp in insect cells has opened a unique opportunity to manipulate viral genome and study the role of viral encoded proteins in host-pathogen interactions. In addition, this has opened a new paradigm to use shrimp viruses as vector to deliver foreign nucleic acid such as RNAi molecules.

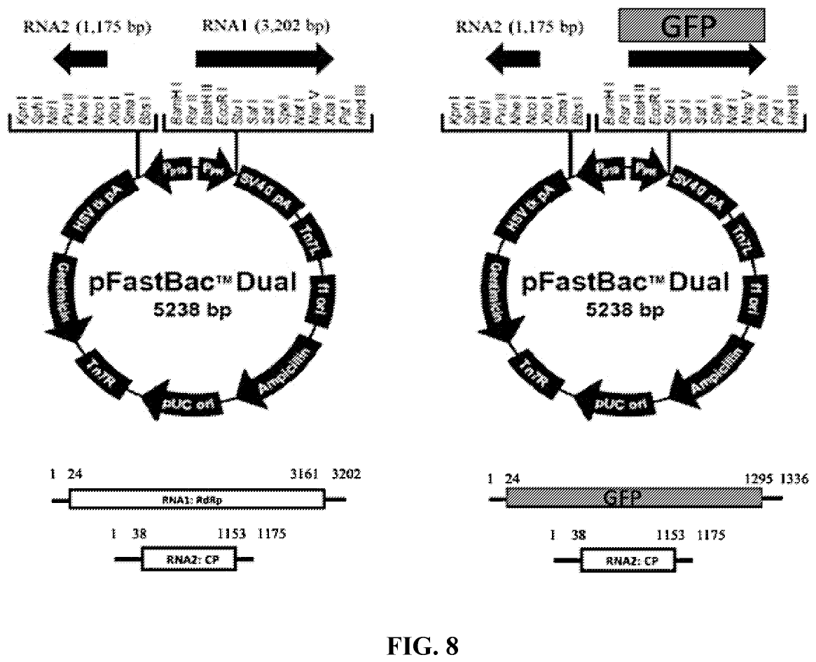

To investigate the potential of recombinant MrNV as a viral vector to deliver a foreign gene in shrimp cells, MrNV RNA1 ORF was replaced by green fluorescent protein (GFP) gene (FIG. 8). From here forth, the rBV carrying shrimp vector MrNV expressing GFP construct shall be designated as “rBV-MrNV-GFP.”

### Expression of a Reporter Gene Using a Shrimp Nodavirus Vector in Sf9 Cells.

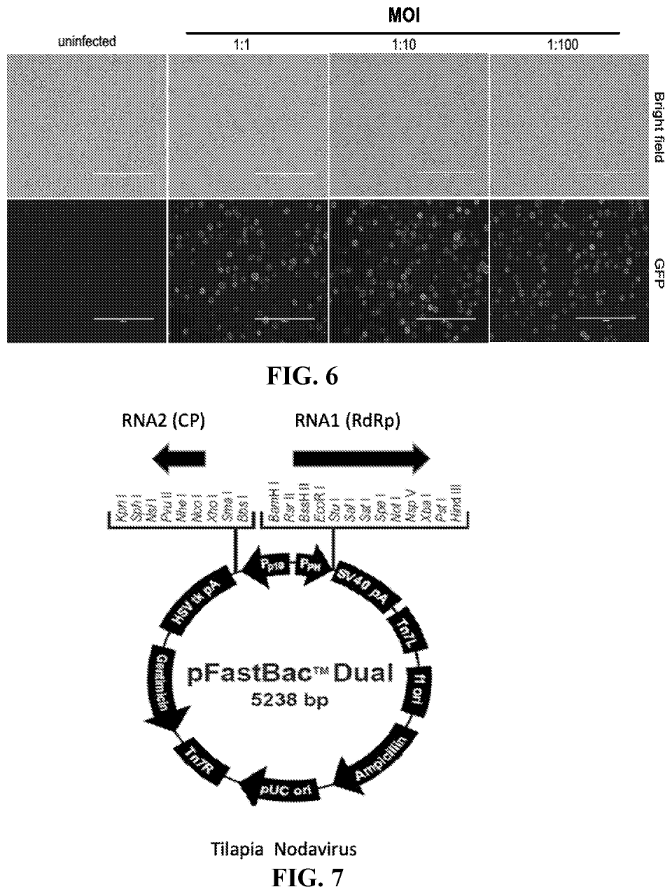

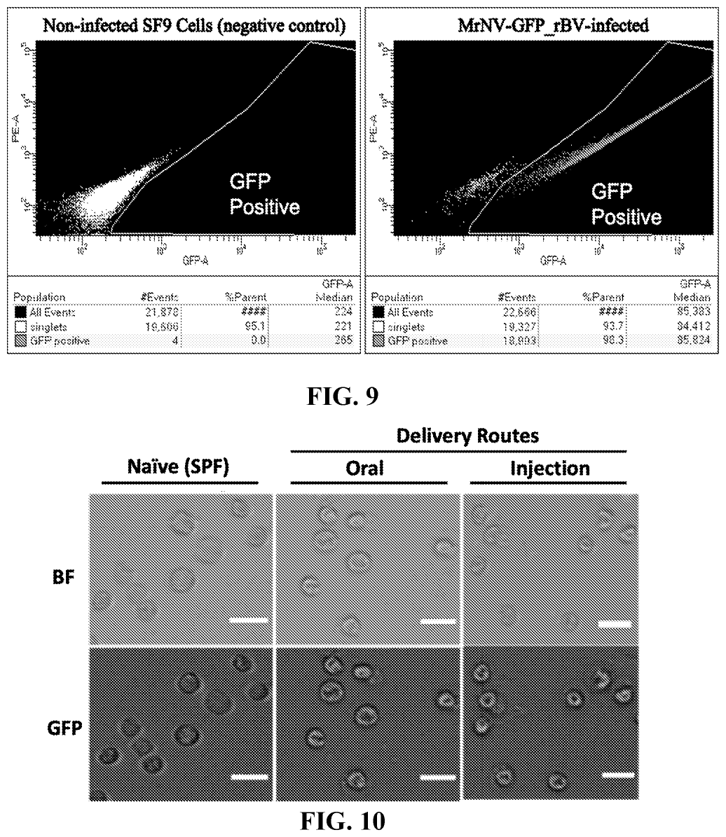

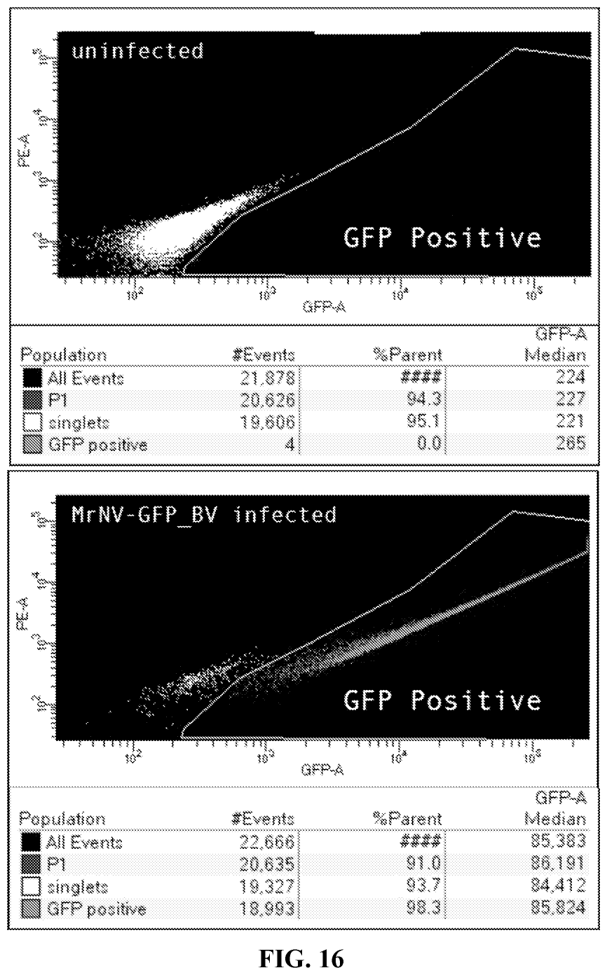

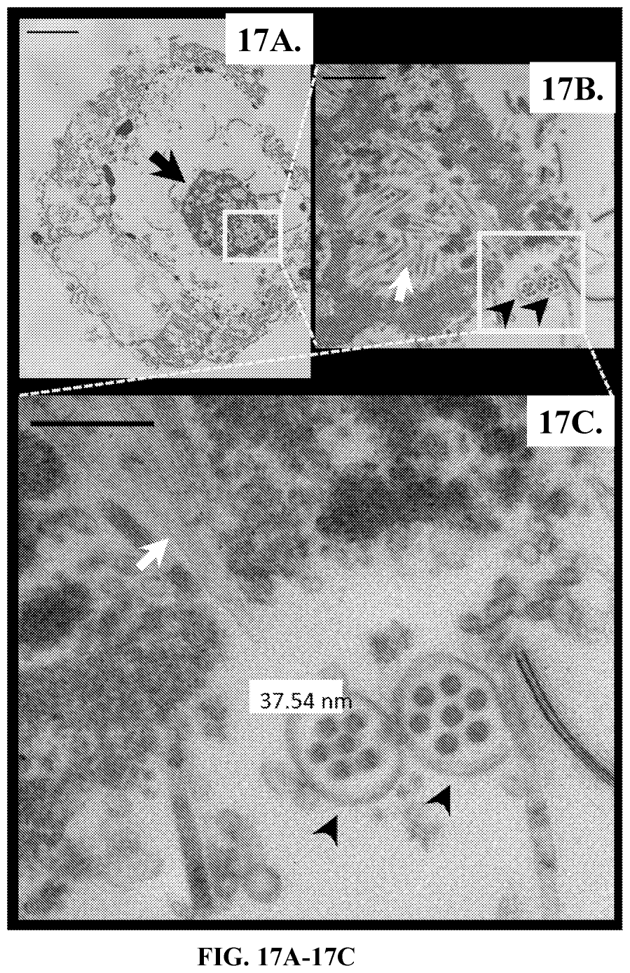

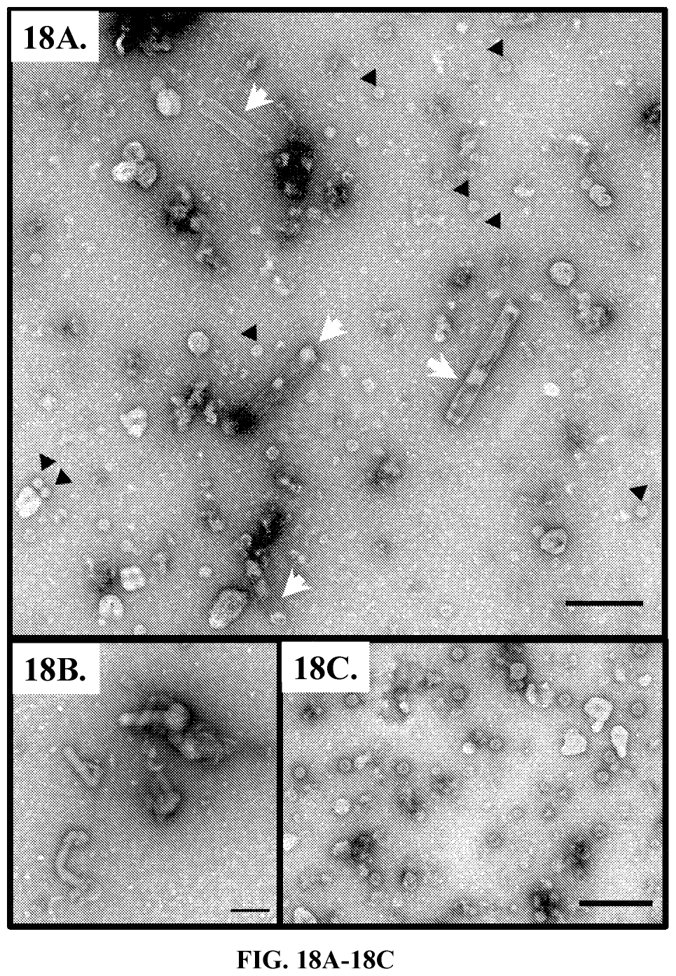

To demonstrate the efficiency of MrNV vector in delivering a foreign gene in shrimp and its successful expression, SF9 cells were inoculated with the rBV carrying GFP gene (rBV-MrNV-GFP) and analyzed for GFP expression by fluorescence microscopy. Results show GFP expression at multiplicity of infection (MOI) of 1, 10, and 100 at Day 1, Day 2 and day 5 post-inoculation (FIG. 6). The efficiency of the viral delivery is demonstrated by flow cytometry analysis. The data show 98% of the total cells are transfected and successfully expressing GFP gene at 2 days post-inoculation (FIGS. 9 and 16). FIGS. 17A-17C and 18A-18C demonstrate production of MrNV-GFP in SF9 cells by a baculovirus expression system and separation of Baculovirus and MrNVcp-GFP.

### Delivery of a GFP Reporter Gene in Shrimp Using a Nodaviral Vector by Injection and Feeding.

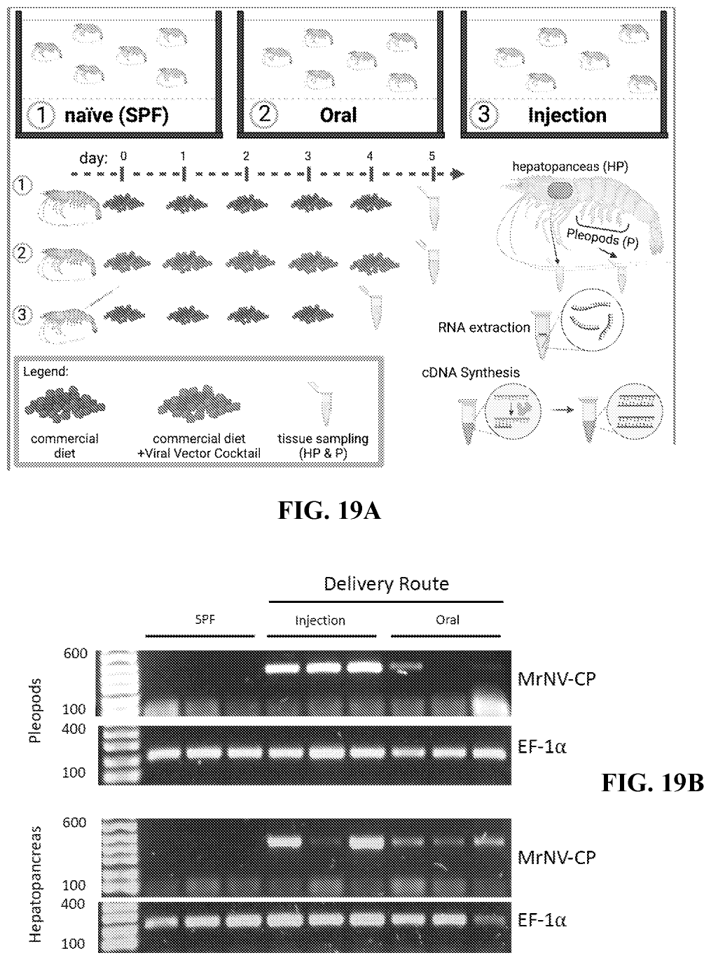

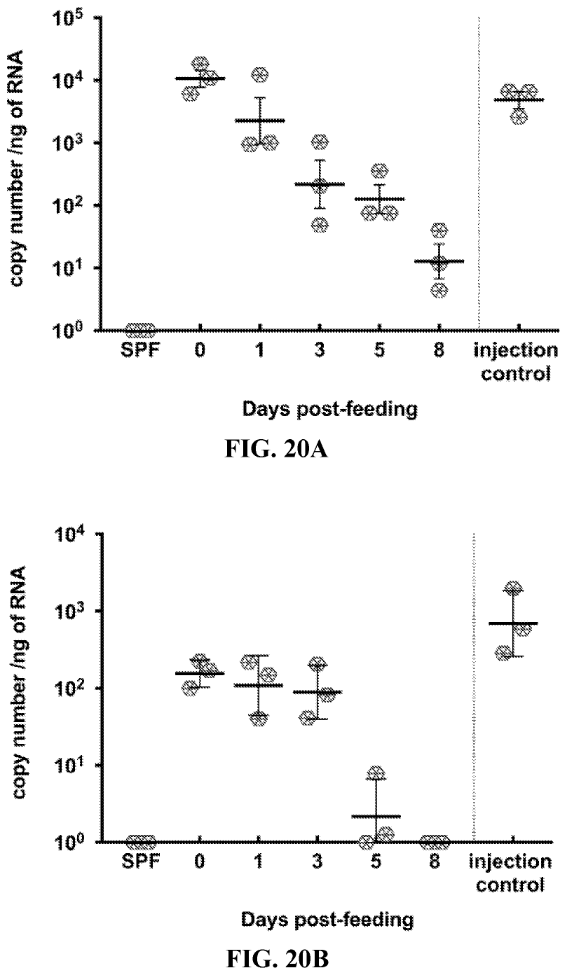

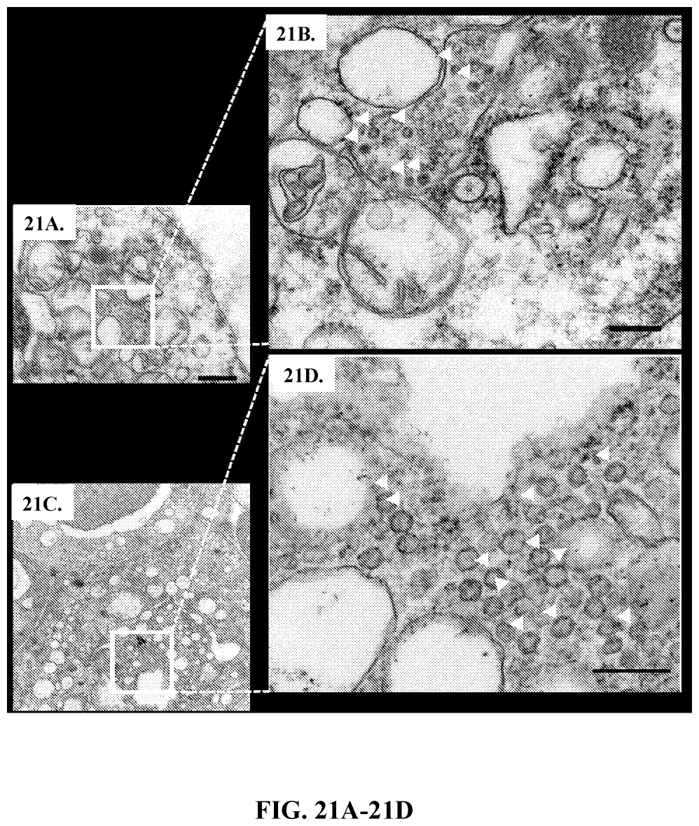

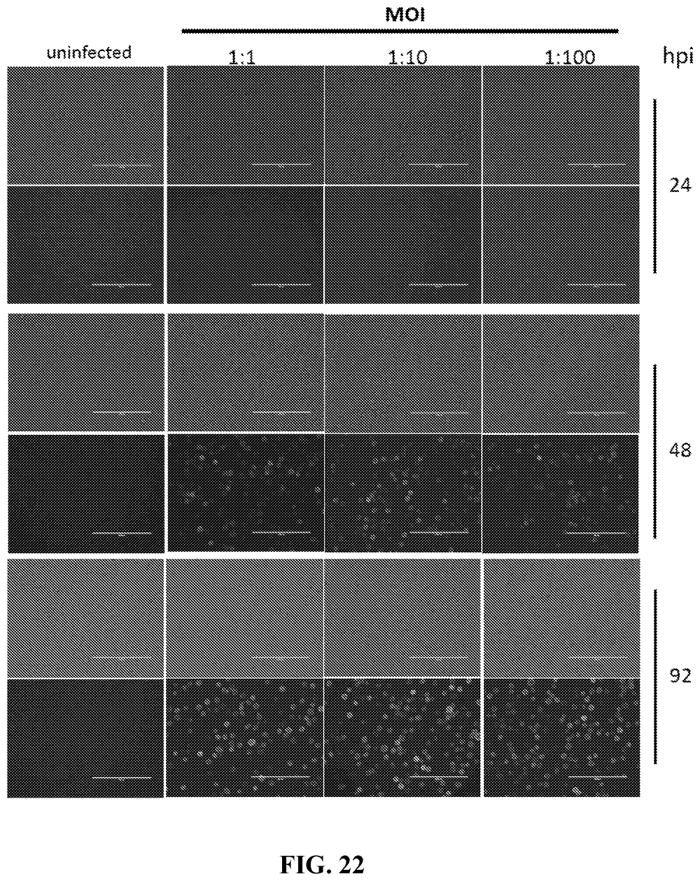

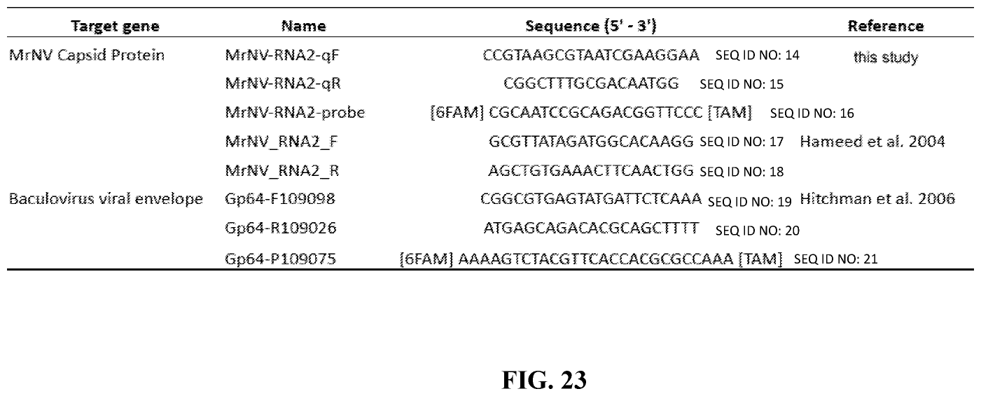

It is desirable that a baculovirus carrying a shrimp viral vector with GFP marker (rBV-MrNV-GFP) should be able to successfully deliver and express GFP gene in shrimp cells and/or able to deliver the payload (GFP in this case), preferably via the oral route in shrimp. The utility of the shrimp viral vector in delivering the GFP gene was examined first by direct inoculation of the vector by intramuscular injection, and then by oral feeding. For this, three groups comprising five individual SPF P. vannamei shrimp (wt. ˜2.5-3 g size) were generated: the first group was injected once with 50 μl of ˜1×10⁷ pfu/mL of rBV-MrNV-GFP diluted with PBS. The second group was fed with commercial shrimp feeds soaked with rBV-MrNV-GFP-infected SF9 cell suspension for 15 min prior to feeding at 5% of the biomass for 5 consecutive days (about 0.6 g feed per day that was soaked in 500 μl of SF9 cell suspension containing ≥1×10⁷ pfu/mL of rBV-MrNV-GFP). The third group were fed with the same commercial feed (without the Sf9 cell homogenate) and served as the negative/naïve control. Hemolymph were drawn from all groups after 5 days, hemocytes were separated by centrifugation and seeded in 24-well plate with L-15 media, then viewed under fluorescent microscope. Quantitative RT-PCR was also used to observe infection characteristics. Primers for the PCR-based techniques for detecting MrNV capsid and envelope proteins are shown in e.g., FIG. 23. See e.g., FIG. 19A for experimental strategy. Results show that hemocytes from groups inoculated rBV-MrNV-GFP, both by injection and oral feeding, were expressing GFP (FIGS. 10, 19B, 20A-20B, 21A-21D, and 22). These results at least demonstrate that a baculovirus based shrimp nodaviral vector can effectively deliver a foreign gene, GFP in vivo by injection and by oral route.

Construction of a Shrimp Viral Vector Containing Hairpin RNA (hRNA) Targeted Against WSSV Structural and Non-Structural Genes

A schematic representation of developing engineered shrimp delivery vectors and vaccines or other nucleic acid based therapeutics based upon such vectors is shown in e.g., FIGS. 1 and 24. To determine the feasibility of delivering hairpin RNA (hRNA) using a shrimp viral vector, six WSSV genes can be targeted. These genes are known targets for WSSV RNAi-experiment in published literatures. It is worth noting that the RNAi sequence was delivered as in vitro transcribed dsRNA via intramuscular injection to knock out the cognate genes [30, 35, 36, 38]. These gene were chosen considering their temporal nature of expression and also included WSSV structural and non-structural genes. These genes include immediate-early genes (ie1), early transcribed DNA polymerase (dnapol) and ribonuclease reductase small subunit 1 (rr1), major late structural proteins VP26 and VP28 and a non-structural protein VP9 genes. Hairpin RNA can be designed using a computer program [RNAiFold 2.0: a web server and software to design custom and Rfam-based RNA molecules] [43-44]. For each WSSV gene, three hRNA will be computationally designed (approximately 100 nucleotide in size), custom synthesized and cloned in tandem replacing the GFP gene in the rBV-MrNV-GFP construct. Recombinant baculovirus expressing MrNV containing hRNA sequence can be generated following a method that is essentially same as making infectious MrNV clone, as described previously herein. Altogether six rBV-MrNV_{hRNA (1 to 6)} will be made. In addition, a rBV-MrNV carrying scrambled hRNA can be designed computationally, custom synthesized and cloned replacing GFP in rBV-MrNV backbone that will serve as a negative control (rBV-MrNV_{Con}). All hRNA will be custom synthesized (GenScript, Piscataway, NJ), and to expedite the project, three rBV-MrNV and the control baculovirus (rBV-MrNV_{Con}) can be custom made using GenScript Baculovirus expression service. The remaining three rBV-MrNV can be cloned and expressed in Sf9 cells in-house.

Six rBV-MrNV_{hRNA (1 to 6)} and the rBV-MrNV_{Con} can be purified from Sf9 cells using a density gradient virus purification method [42], and transmission electron microscopy (TEM) can be performed in a suitable facility, e.g., The University of Arizona Core Imaging Facility.

Total DNA and total RNA can be isolated from purified virus (rMrNV_{hRNA} and the corresponding baculoviruses) and used in conventional PCR/RT-PCR to determine the size of the construct using primers flanking just outside the hRNA sequence. This can allow for determination that the hRNA sequence containing three targets for each of the six genes is present in the baculovirus as well as the in the rMrNV, and the hRNA sequence has not been excised as baculovirus replicates in Sf9 cells. Applicant will also perform real-time PCR/RT-PCR using the same total DNA and total RNA to further verify the sequence integrity in the stem loop structure of the hairpin using two to three sets of primers for each virus.

Titer can be determined using an appropriate antibody. Wild type MrNV can be used to make polyclonal antibodies using conventional antibody manufacturing and purification techniques. These antibodies can be used to determine MrNV titer in Sf9 cells.

Confirmation of Expression of hRNA Delivered Using a Shrimp Viral Vector In Vitro (Using Sf9 Cells and Shrimp Primary Hemocytes) and In Vivo (Live Shrimp).

To confirm the expression of hRNA targeting WSSV genes delivered by shrimp viral vector, SF9 cells and shrimp primary hemocyte culture can be experimentally inoculated using purified rBV viral fractions as well as the purified rBV-MrNV_{hRNA (1 to 6)} and the control virus preparation. Altogether there will be 14 viral preparations purified from seven constructs (six hRNA constructs and one control).

In vitro expression assay: SF9 cells and hemocytes drawn from SPF P. vannamei shrimp can be seeded in 24-well culture plate using ESF 921™ Insect Cell Culture Medium and following published protocols [45, 46]. The cells can be inoculated using different MOIs (1:1, 1:10, and 1:100). Infected SF9 cells and shrimp primary hemocytes can be collected at 24, 48, and 72 post-inoculation for RNA extraction and cDNA synthesis.

Relative expression of hRNA can be measured by real-time RT-PCR. The primers to measure the expression of hRNA will be same as previously described (to determine the stability of the hRNA sequence in the recombinant virus). The expression levels of hRNA can be normalized to internal control gene elongation factor-1α for both Sf9 cells and shrimp. Shrimp elongation factor-1α (EF-1α) primer sequence is same as published earlier [47], and the primers to amplify Sf9 (EF-1α) can be designed based on the NCBI entry, XM_035588389.1.

In vivo expression assay: Fourteen viral preparations will be taken to feed live SPF P. vannamei shrimp (wt. range 4-5 gm). Each animal will be fed at 5% biomass per day using feed pre-soaked in viral solution containing 1×10⁷ particles/ml purified virus for 15 min prior to feeding, as performed for GFP oral delivery (FIG. 10). There will be 10 animals/per tank for each virus containing feed. Animals fed diet soaked in rBV-MrNV containing scrambled RNA sequence will serve as a negative control.

The animals utilized for this can be SPF (specific pathogen free) P. vannamei and stocked at 10 animals per tank in a 90 L containing artificially sea water routinely used in Aquaculture Pathology Laboratory for animal husbandry. The commercial pelleted shrimp diet (Rangen, 40% protein) can be used for feeding for 5 days. All aquaria can be outfitted with a pre-conditioned biological filter and adequate aeration. On Day 07, hemolymph will be withdrawn for hRNA expression analysis, and the sacrificed animals can be stored at −80° C. Other tissues like muscle tissue, hepatopancreas etc., samples can be collected, stored, and available for analysis.

Based on the rBV-MrNV-GFP expression data as previously described, it is expected that by feeding rBV-MrNV_{hRNA (1 to 6)} for 05 days, we should be able to detect hRNA expression, if the hRNA is not degraded to the extent that primer binding site in the isolated RNA is destroyed. The real-time RT-PCR based assay to measure the expression of hRNA can be same as described in the in vitro assay section above.

Determining the Efficacy of Shrimp Viral Vector in Delivering hRNA Via Diet and Protecting Shrimp Against WSD.

hRNA candidates whose levels are successfully detected in the hemocytes upon delivering the corresponding viral construct through diet can be used in a WSD challenge to determine their efficacy. An exemplary challenge to determine efficacy of any of the vectors, treatments, and/or preventatives described and/or demonstrated elsewhere herein are now discussed.

Animals & Treatments: For experimental challenge, SPF P. vannamei can be obtained from a certified vendor in the US. The animals can be kept in a 1000L tank with 60 animals per tank (wt. range 4-5 gm). There can be two 1000L tank per treatment for a total of 08 tanks for hRNA treatments, 02 positive and 02 negative control tanks. All aquaria can be outfitted with a pre-conditioned biological filter and adequate aeration.

There can be n+1 viral treatments: n hRNA expressing candidate viruses and an additional treatment containing equal proportions of the three recombinant viruses. Without being bound by theory the rationale of combining multiple viruses is based on the fact that gene knockout efficiency is higher when multiple targets for multiple genes are combined than a single target-single gene.

An additional negative control virus treatment can be included. Animals in all treatments will be challenged with WSSV orally. The final additional treatment can include animals those will be fed the negative control virus containing diet but remain unchallenged.

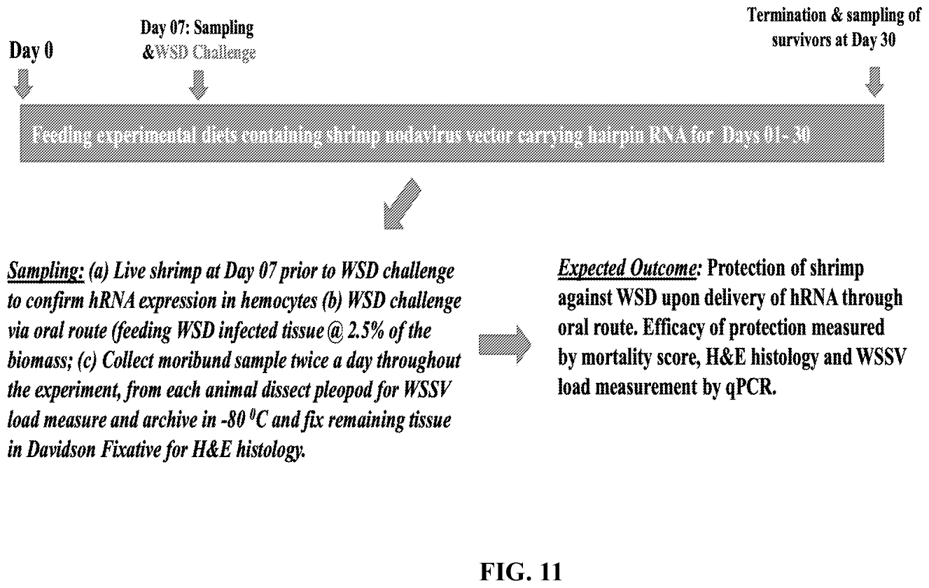

Preparation of feed and feeding regime: The method can be essentially same as previously described. Briefly, animals can be fed at 5% biomass per day with shrimp diet (Rangen, 40% protein) that can be soaked in Sf9 cell dell-derived homogenate containing rBV and rMrNV_{hRNA} throughout the duration of the test (FIG. 11). The quantity of virus to be taken in mixing with the feed can be determined essentially based on the on data obtained from determining the expression of candidate constructs. Without being bound by theory, it is likely that the Sf9 cell culture derived homogenate can contain about ≥1×10⁷ of each of rBV particles/ml as well as rBV-MrNV_{hRNA}. Control diet can be soaked in rBV-MrNV_{Con} Sf9 cell homogenate.

WSD Challenge: Animals can be fed experimental diets for 06 days. On Day 07, hemolymph can be withdrawn from 05 animals/tank, and animals can be sacrificed upon drawing hemolymph to examine the level of hRNA. Hemolymph and the animals will be stored at −80° C. for any future need, like examining the hRNA expression in other tissues like muscle tissue, hepatopancreas or examining transcriptome profile in hRNA fed vs. unfed animals etc. The remaining animals can be taken for WSSV challenge. Animals in all but the negative control can be fed WSSV positive tissue (Isolate CN 95) at a rate of 2.5% of the biomass in the tank. This protocol of WSSV challenge is routinely followed for screening genetic lines, evaluating therapeutics, and it gives ˜80% mortality. Conducting WSSV oral challenge using less than 2.5% infected tissue does not give very predictable mortality and that is why this dose of viral inoculum is used. Both the therapeutic and the WSSV inoculum can be administered following an oral route which mimics a natural route of feeding and WSSV transmission.

Data collection: Overall, three sets of data can be generated form the WSD challenge study: (1) Mortality data will be recorded throughout the WSD challenge study, (2) H&E histopathology can. be performed on the moribund and surviving animals for each treatment (e.g., N=20 samples/treatment, 10 per tank), and (3) WSSV load can be determined using an OIE-recommended qPCR protocol using the moribund and surviving animals from each treatment (e.g., N=20 animals/treatment, 10 per tank). All moribund samples and the survivors at termination will be archived after taking samples for histopathology and PCR for any future need. (4) As subset of animals (N=5) will be taken from Day 07 sampling time point will be taken for the detection of MrNV by immune histochemistry using anti MrNV polyclonal antibody and hRNA by in situ hybridization.

All statistical analyses can be conducted using JMP 14.0 (SAS Corporation). Survivorship upon WSSV challenge will be analyzed using a Kaplan-Meier with log-rank Chi-Square statistics, Cox Proportional Hazard tests, and risk ratios (p<0.05) to test for differences in survival among different treatment groups will be performed. Differences in mean viral loads among WSSV challenged treatments will be evaluated using least square regression Tukey HSD.

### REFERENCES RELATED TO EXAMPLE 1

1. Flegel, T. W. Historic emergence, impact and current status of shrimp pathogens in Asia. J. Invertebr. Pathol. 2012, 110, 166-173, doi:10.1016/j.jip.2012.03.004.
2. Lightner, D. V; Redman, R. M.; Pantoja, C. R.; Tang, K. F. J.; Noble, B. L.; Schofield, P.; Mohney, L. L.; Nunan, L. M.; Navarro, S. A. Historic emergence, impact and current status of shrimp pathogens in the Americas. J. Invertebr. Pathol. 2012, 110, 174-183, doi:10.1016/j.jip.2012.03.006.
3. Treece, G. Seafood Watch Whiteleg Shrimp; San Diego, 2014;
4. Kiyoshi, I.; Miwa, S.; Oseko, N.; Nakano, H.; Kimura, T.; Momoyama, K.; Hiraoka, M. Mass Mortalities of Cultured Kuruma Shrimp Penaeus japonicus Evidence in Japan in 1993: Electron Microscopic of the Causative Virus. Fish Pathol. 1994, 2, 149-158.
5. Flegel, T. W. Detection of major penaeid shrimp viruses in Asia, a historical perspective with emphasis on Thailand. Aquaculture 2006, 258, 1-33, doi:10.1016/j.aquaculture.2006.05.013.
6. OIE Manual of Diagnostic Tests for Aquatic Animals; Fourth Edi.; OFFICE INTERNATIONAL DES EPIZOOTIES: Paris, France, 2003; Vol. 33; ISBN 9290445637.
7. Sinchez-Paz, A. White spot syndrome virus: An overview on an emergent concern. Vet. Res. 2010, 41, doi:10.1051/vetres/2010015.
8. Shirley, M.; Extension), (LSU AgCenter Louisiana Crawfish: White Spot Virus in Ponds.
9. JOHNSON, C. (Associated P. Crawfish concerns: “White spot” virus hitting Louisiana ponds, threatening industry.
10. Baumgartner, W. A.; Hawke, J. P.; Bowles, K.; Varner, P. W.; Hasson, K. W. Primary diagnosis and surveillance of white spot syndrome virus in wild and farmed crawfish (Procambarus clarkii, P. zonangulus) in Louisiana, USA. Dis. Aquat. Organ. 2009, 85, 15-22, doi:10.3354/dao02051.
11. Food and Agriculture Organization-2019. GLOBE FISH HIGHLIGHTS—A QUARTERLY UPDATE ON WORLD SEAFOOD MARKETS Available online: http://www.fao.org/documents/card/en/c/ca5870en/
12. Hans, C. White Spot Outbreak in Shrimp in Hawaii Available online: http://www.thefishsite.com/fishnews/17505/white-spot-outbreak-in-shrimp-in-hawaii.
13. Lo, C. F. Virus Taxonomy: IXth Report of the International Committee on Taxonomy of Viruses. In Virus Taxonomy; King, A., Adams, M., Carstens, E., Lefkowitz, E. J., Eds.; Elsevier: Amsterdam, 2011; pp. 229-234.
14. Tsai, J.-M.; Wang, H.-C.; Leu, J.-H.; Wang, A. H.-J.; Zhuang, Y.; Walker, P. J.; Kou, G.-H.; Lo, C.-F. Identification of the Nucleocapsid, Tegument, and Envelope Proteins of the Shrimp White Spot Syndrome Virus Virion. J. Virol. 2006, 80, 3021-3029, doi:10.1128/jvi.80.6.3021-3029.2006.
15. Tsai, J.-M.; Wang, H.-C.; Leu, J.-H.; Hsiao, H.-H.; Wang, A. H.-J.; Kou, G.-H.; Lo, C.-F. Genomic and Proteomic Analysis of Thirty-Nine Structural Proteins of Shrimp White Spot Syndrome Virus. J Virol. 2004, 78, 11360-11370, doi:10.1128/jvi.78.20.11360-11370.2004.
16. Chang, Y.-S.; Liu, W.-J.; Chou, T.-L.; Lee, Y.-T.; Lee, T.-L.; Huang, W.-T.; Kou, G.-H.; Lo, C.-F. Characterization of White Spot Syndrome Virus Envelope Protein VP51A and Its Interaction with Viral Tegument Protein VP26. J Virol. 2008, 82, 12555-12564, doi:10.1128/jvi.01238-08.
17. Flegel, T. W. Special topic review: Major viral diseases of the black tiger prawn (Penaeus monodon) in Thailand. World J Microbiol. Biotechnol. 1997, 13, 433-442, doi:oi.org/10.1023/A:1018580301578.
18. Lo, C. F.; Ho, C. H.; Peng, S. E.; Chen, C. H.; Hsu, H. C.; Chiu, Y. L.; Chang, C. F.; Liu, K. F.; Su, M. Sen; Wang, C. H.; et al. White spot syndrome baculovirus (WSBV) detected in cultured and captured shrimp, crabs and other arthropods. Dis. Aquat. Org. 1996, 27, 215-225, doi:10.3354/dao027215.
19. Lo, C. F.; Ho, C. H.; Chen, C. H.; Liu, K. F.; Chiu, Y. L.; Yeh, P. Y.; Peng, S. E.; Hsu, H. C.; Liu, H. C.; Chang, C. F.; et al. Detection and tissue tropism of white spot syndrome baculovirus (WSBV) in captured brooders of Penaeus monodon with a special emphasis on reproductive organs. Dis. Aquat. Org. 1997, 30, 53-72, doi:10.3354/dao030053.
20. Vlak, J. M. VIIIth report of the International Committee on Taxonomy of Viruses. In VIIIth report of the International Committee on Taxonomy of Viruses; Fauquet, C., Mayo, M., Maniloff, J., Desselberger, U., Ball, L., Eds.; Elsevier: Amsterdam, 2004; pp. 187-192.
21. Van Hulten, M. C. W.; Witteveldt, J.; Snippe, M.; Vlak, J. M. White spot syndrome virus envelope protein VP28 is involved in the systemic infection of shrimp. Virology 2001, 285, 228-233, doi:10.1006/viro.2001.0928.
22. Robalino, J.; Payne, C.; Parnell, P.; Shepard, E.; Grimes, A. C.; Metz, A.; Prior, S.; Witteveldt, J.; Vlak, J. M.; Gross, P. S.; et al. Inactivation of White Spot Syndrome Virus (WSSV) by normal rabbit serum: Implications for the role of the envelope protein VP28 in WSSV infection of shrimp. Virus Res. 2006, 118, 55-61, doi:10.1016/j.virusres.2005.11.011.
23. Sritunyalucksana, K.; Wannapapho, W.; Lo, C. F.; Flegel, T. W. PmRab7 Is a VP28-Binding Protein Involved in White Spot Syndrome Virus Infection in Shrimp. J Virol. 2006, 80, 10734-10742, doi:10.1128/jvi.00349-06.
24. Siomi, H.; Siomi, M. C. On the road to reading the RNA-interference code. Nature 2009, 457, 396-404, doi:10.1038/nature07754.
25. Yao, X.; Wang, L.; Song, L.; Zhang, H.; Dong, C.; Zhang, Y.; Qiu, L.; Shi, Y.; Zhao, J.; Bi, Y. A Dicer-1 gene from white shrimp Litopenaeus vannamei: Expression pattern in the processes of immune response and larval development. Fish Shellfish Immunol. 2010, 29, 565-570, doi:10.1016/j.fsi.2010.05.016.
26. Chen, Y. H.; Jia, X. T.; Zhao, L.; Li, C. Z.; Zhang, S.; Chen, Y. G.; Weng, S. P.; He, J. G. Identification and functional characterization of Dicer2 and five single VWC domain proteins of Litopenaeus vannamei. Dev. Comp. Immunol. 2011, 35, 661-671, doi:10.1016/j.dci.2011.01.010.
27. Su, J.; Oanh, D. T. H.; Lyons, R. E.; Leeton, L.; van Hulten, M. C. W.; Tan, S. H.; Song, L.; Rajendran, K. V.; Walker, P. J. A key gene of the RNA interference pathway in the black tiger shrimp, Penaeus monodon: Identification and functional characterisation of Dicer-1. Fish Shellfish Immunol. 2008, 24, 223-233, doi:10.1016/j.fsi.2007.11.006.
28. Yang, L.; Li, X.; Jiang, S.; Qiu, L.; Zhou, F.; Liu, W.; Jiang, S. Characterization of Argonaute2 gene from black tiger shrimp (Penaeus monodon) and its responses to immune challenges. Fish Shellfish Immunol. 2014, 36, 261-269, doi:10.1016/j.fsi.2013.11.010.
29. Huang, T.; Zhang, X. Host defense against DNA virus infection in shrimp is mediated by the siRNA pathway. Eur. J Immunol. 2013, 43, 137-146, doi:10.1002/eji.201242806.
30. Westenberg, M.; Heinhuis, B.; Zuidema, D.; Vlak, J. M. siRNA injection induces sequence-independent protection in Penaeus monodon against white spot syndrome virus. Virus Res. 2005, 114, 133-139, doi:10.1016/j.virusres.2005.06.006.
31. Yodmuang, S.; Tirasophon, W.; Roshorm, Y.; Chinnirunvong, W.; Panyim, S. YHV-protease dsRNA inhibits YHV replication in Penaeus monodon and prevents mortality. Biochem. Biophys. Res. Commun. 2006, 341, 351-356, doi:10.1016/j.bbrc.2005.12.186.
32. Tirasophon, W.; Roshorm, Y.; Panyim, S. Silencing of yellow head virus replication in penaeid shrimp cells by dsRNA. Biochem. Biophys. Res. Commun. 2005, 334, 102-10⁷, doi:10.1016/j.bbrc.2005.06.063.
33. Tirasophon, W.; Yodmuang, S.; Chinnirunvong, W.; Plongthongkum, N.; Panyim, S. Therapeutic inhibition of yellow head virus multiplication in infected shrimps by YHV-protease dsRNA. Antiviral Res. 2007, 74, 150-155, doi:10.1016/j.antiviral.2006.11.002.
34. Liu, W. J.; Chang, Y. S.; Wang, C. H.; Kou, G. H.; Lo, C. F. Microarray and RT-PCR screening for white spot syndrome virus immediate-early genes in cycloheximide-treated shrimp. Virology 2005, 334, 327-341, doi:10.1016/j.virol.2005.01.047.
35. Sanjuktha, M.; Stalin Raj, V. S.; Aravindan, K.; Alavandi, S. V.; Poornima, M.; Santiago, T. C. Comparative efficacy of double-stranded RNAs targeting WSSV structural and nonstructural genes in controlling viral multiplication in Penaeus monodon. Arch. Virol. 2012, 157, 993-998, doi:10.1007/s00705-012-1258-2.
36. Alenton, R. R. R.; Kondo, H.; Hirono, I.; Maningas, M. B. B. Gene silencing of VP9 gene impairs WSSV infectivity on Macrobrachium rosenbergii. Virus Res. 2016, 214, 65-70, doi:10.1016/j.virusres.2016.01.013.
37. Itsathitphaisarn, O.; Thitamadee, S.; Weerachatyanukul, W.; Sritunyalucksana, K. Potential of RNAi applications to control viral diseases of farmed shrimp. J. Invertebr. Pathol. 2017, 147, 76-85, doi:10.1016/j.jip.2016.11.006.
38. Sarathi, M.; Simon, M. C.; Venkatesan, C.; Hameed, A. S. S. Oral administration of bacterially expressed VP28dsRNA to protect Penaeus monodon from white spot syndrome virus. Mar. Biotechnol. 2008, 10, 242-249, doi:10.1007/s10126-007-9057-6.
39. George, S. K.; Kaizer, K. N.; Betz, Y. M.; Dhar, A. K. Multiplication of Taura syndrome virus in primary hemocyte culture of shrimp (Penaeus vannamei). J Virol. Methods 2011, 172, 54-59, doi:10.1016/j.jviromet.2010.12.020.
40. Bonami, J. R.; Sri Widada, J. Viral diseases of the giant fresh water prawn Macrobrachium rosenbergii: A review. J. Invertebr. Pathol. 2011, 106, 131-142, doi:10.1016/j.jip.2010.09.007.
41. Gangnonngiw, W.; Bunnontae, M.; Phiwsaiya, K.; Senapin, S.; Dhar, A. K. In experimental challenge with infectious clones of Macrobrachium rosenbergii nodavirus (MrNV) and extra small virus (XSV), MrNV alone can cause mortality in freshwater prawn (Macrobrachium rosenbergii). Virology 2020, 540, 30-37, doi:10.1016/j.virol.2019.11.004.
42. Dhar, Arun K., 2011. Expression of positive sense single stranded RNA virus and uses thereof. US provisional patent application. Publication number: 20130195914. https://patents.justia.com/patent/20130195914
43. Moore, Chris B.; Guthrie, Elizabeth H.; Huang, Max Tze-Han; Taxman, D. J. Short hairpin RNA therapeutics: function, design, and delivery. Preface. Methods Mol. Biol. 2010, 629, 1-15, doi:10.1007/978-1-60761-657-3.
44. Garcia-Martin, J. A.; Dotu, I.; Clote, P. RNAiFold 2.0: A web server and software to design custom and Rfam-based RNA molecules. Nucleic Acids Res. 2015, 43, W513-W521, doi:10.1093/nar/gkv460.
45. George, S. K., Kaizer, K. N., Betz, Y. M., and Dhar, A. K. 2011. Multiplication of Taura syndrome virus in the primary hemocyte culture of shrimp (Penaeus vannamei). Journal of Virological Methods, 2011, 172: 54-59.
46. George, S. K., and Dhar, A. K. An improved method of cell culture system from eye stalk, hepatopancreas, muscle, ovary and hemocytes of Penaeus vannamei. In Vitro Cellular & Developmental Biology—Animal, 2010 46: 801-810.
47. Dhar, A. K., Bowers, R. A., Licon, K. S., Veaze, G., and Reads, B. Validation of reference genes for quantitative measurement of immune gene expression in shrimp. Molecular Immunology, 2009, 46: 1688-95.

### Example 2

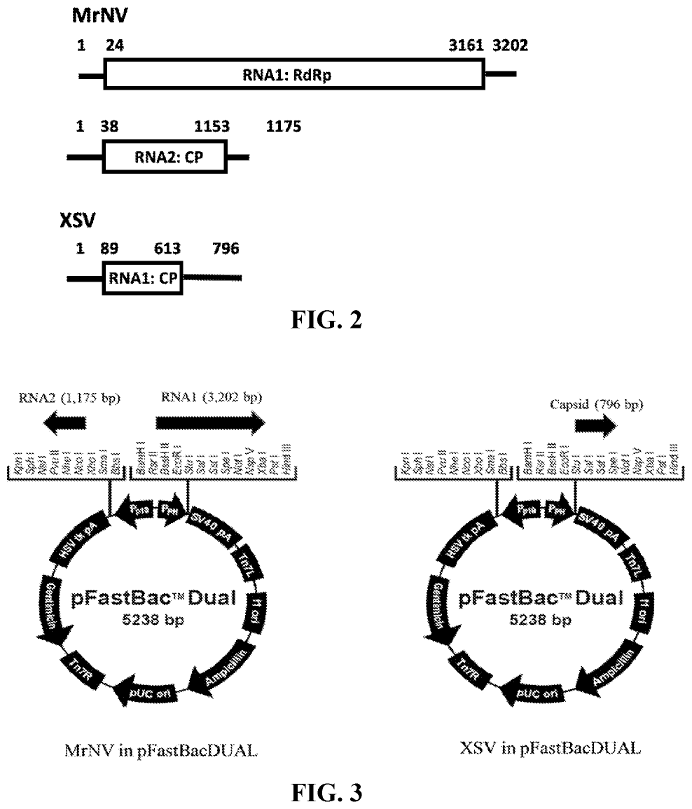

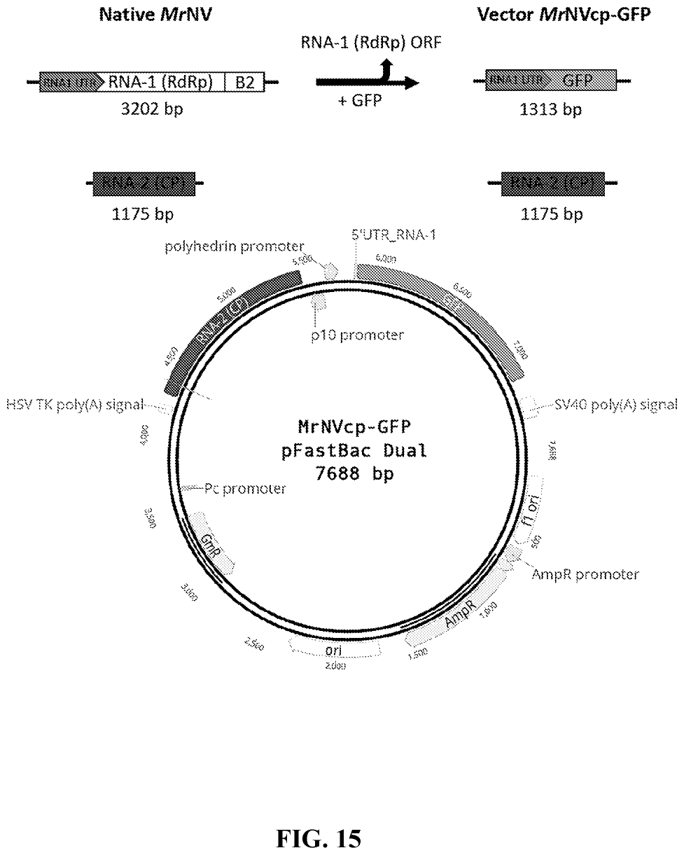

FIG. 1 shows a schematic overview of developing oral vaccines/therapeutics against diseases in aquatic animals using a Nodavirus-based engineered viral vector based system. FIG. 2 shows a schematic representation of the genome organization of Macrobrachium rosenbergii nodavirus (MrNV) and extra small virus (XSV) infecting freshwater prawn and penaeid shrimp. The numbers above the lines represent nucleotide numbers of the viral RNA. The box indicates an open reading frame encoding RNA-dependent RNA polymerase (RdRp) in RNA1 (nucleotides 24 to 3161) and capsid protein (CP) gene (nucleotides 38 to 1153) in RNA2 segment of the viral genome. The genome of XSV encodes a capsid protein only (nucleotides 89 to 613). FIG. 3 shows a genomic map of infectious cDNA clones of MrNV and XSV. The full-length RNA1 and RNA 2 of MrNV (Left Panel) and full-length genome of XSV were clones in a baculovirus pFastBacDUAL vector (Gangnonngiw et al., Virology 2020, 540, 30-37). See also FIG. 15, which shows another exemplary MrNVcp-GFP viral vector construct.

FIG. 4 shows transmission electron micrographs of negatively stained viral preparations of MrNV (left), XSV (middle) and recombinant baculovirus (right) purified from Sf9 cells (Gangnonngiw et al., 2020).

FIG. 5 shows a genomic map of infectious recombinant MrNV where the open reading frame (ORF) representing the RNA-dependent RNA polymerase (RdRp) gene in RNA1 was replaced with a marker gene, Green Fluorescent Protein (GFP) gene. In the bottom of the panel, a schematic representation of the GFP gene construct is shown. The numbers on the schematic represent nucleotide number. For example, in MrNV RNA 1, nucleotides 1 to 23 represent the 5′-end non-coding region, nucleotides 24 to 1295 represent GFP ORF and nucleotides 1295 through 1336 indicate 3′-end non-coding region of MrNV RNA1. In RNA 2, nucleotides 1 to 37 represent the 5′-end non-coding region, nucleotides 38 to 1153 represent GFP ORF and nucleotides 1154 to 1175 indicate the 3′-end non-coding region of the RNA. FIG. 6 shows GFP expression in SF9 cells 5 days post-inoculation with rBV_MrNV-GFP. Upper panel showing bright field images (BF) and on the lower, GFP-positive cells. Scale bars represent 200 μm.

FIG. 7 shows a genomic map of infectious cDNA clones of sevenband grouper nervous necrosis virus (SGNNV). The full-length RNA1 and RNA 2 of SGNNV were cloned under the control of polyhedrin and p10 promoters, respectively in a baculovirus pFastBacDUAL vector. Recombinant baculovirus carrying SGNNV genome is used to infect Sf9 cells to produce infectious cDNA clones of SGNNV.

The nucleic acid sequences below show in Table 1 various components of the engineered nodaviral vectors described and demonstrated herein.

TABLE 1

| Description | SEQ ID NO: |
|---|---|
| pFastBac Dual | SEQ ID NO: 1 |
| GFP sequence-downstream of Pph promoter | SEQ ID NO: 2 |
| Mr-RNA-2-downstream of P10 promoter | SEQ ID NO: 3 |
| Mr-RNA-1-downstream of Pph promoter | SEQ ID NO: 4 |
| Mr-RNA-2-downstream of P10 promoter | SEQ ID NO: 5 |
| MrNV RNA 1 containing GFP sequence-downstream of | SEQ ID NO: 6 |
| Pph promoter | |

>pFastBac Dual (SEQ ID NO: 1)
(SEQ ID NO: 1)
TTCTCTGTCACAGAATGAAAATTTTTCTGTCATCTCTTCGTTATTAATGTTTGTAA
TTGACTGAATATCAACGCTTATTTGCAGCCTGAATGGCGAATGGGACGCGCCCTG
TAGCGGCGCATTAAGCGCGGCGGGTGTGGTGGTTACGCGCAGCGTGACCGCTAC
ACTTGCCAGCGCCCTAGCGCCCGCTCCTTTCGCTTTCTTCCCTTCCTTTCTCGCCA
CGTTCGCCGGCTTTCCCCGTCAAGCTCTAAATCGGGGGCTCCCTTTAGGGTTCCG
ATTTAGTGCTTTACGGCACCTCGACCCCAAAAAACTTGATTAGGGTGATGGTTCA
CGTAGTGGGCCATCGCCCTGATAGACGGTTTTTCGCCCTTTGACGTTGGAGTCCA
CGTTCTTTAATAGTGGACTCTTGTTCCAAACTGGAACAACACTCAACCCTATCTC
GGTCTATTCTTTTGATTTATAAGGGATTTTGCCGATTTCGGCCTATTGGTTAAAAA
ATGAGCTGATTTAACAAAAATTTAACGCGAATTTTAACAAAATATTAACGTTTAC
AATTTCAGGTGGCACTTTTCGGGGAAATGTGCGCGGAACCCCTATTTGTTTATTTT
TCTAAATACATTCAAATATGTATCCGCTCATGAGACAATAACCCTGATAAATGCT
TCAATAATATTGAAAAAGGAAGAGTATGAGTATTCAACATTTCCGTGTCGCCCTT
ATTCCCTTTTTTGCGGCATTTTGCCTTCCTGTTTTTGCTCACCCAGAAACGCTGGT
GAAAGTAAAAGATGCTGAAGATCAGTTGGGTGCACGAGTGGGTTACATCGAACT
GGATCTCAACAGCGGTAAGATCCTTGAGAGTTTTCGCCCCGAAGAACGTTTTCCA
ATGATGAGCACTTTTAAAGTTCTGCTATGTGGCGCGGTATTATCCCGTATTGACG
CCGGGCAAGAGCAACTCGGTCGCCGCATACACTATTCTCAGAATGACTTGGTTGA
GTACTCACCAGTCACAGAAAAGCATCTTACGGATGGCATGACAGTAAGAGAATT
ATGCAGTGCTGCCATAACCATGAGTGATAACACTGCGGCCAACTTACTTCTGACA
ACGATCGGAGGACCGAAGGAGCTAACCGCTTTTTTGCACAACATGGGGGATCAT
GTAACTCGCCTTGATCGTTGGGAACCGGAGCTGAATGAAGCCATACCAAACGAC
GAGCGTGACACCACGATGCCTGTAGCAATGGCAACAACGTTGCGCAAACTATTA
ACTGGCGAACTACTTACTCTAGCTTCCCGGCAACAATTAATAGACTGGATGGAGG
CGGATAAAGTTGCAGGACCACTTCTGCGCTCGGCCCTTCCGGCTGGCTGGTTTAT
TGCTGATAAATCTGGAGCCGGTGAGCGTGGGTCTCGCGGTATCATTGCAGCACTG
GGGCCAGATGGTAAGCCCTCCCGTATCGTAGTTATCTACACGACGGGGAGTCAG
GCAACTATGGATGAACGAAATAGACAGATCGCTGAGATAGGTGCCTCACTGATT
AAGCATTGGTAACTGTCAGACCAAGTTTACTCATATATACTTTAGATTGATTTAA
AACTTCATTTTTAATTTAAAAGGATCTAGGTGAAGATCCTTTTTGATAATCTCATG
ACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAA
ACAAAAAAACCACCGCTACCAGCGGTGGTTTGTTTGCCGGATCAAGAGCTACCA
ACTCTTTTTCCGAAGGTAACTGGCTTCAGCAGAGCGCAGATACCAAATACTGTCC
TTCTAGTGTAGCCGTAGTTAGGCCACCACTTCAAGAACTCTGTAGCACCGCCTAC
ATACCTCGCTCTGCTAATCCTGTTACCAGTGGCTGCTGCCAGTGGCGATAAGTCG
TGTCTTACCGGGTTGGACTCAAGACGATAGTTACCGGATAAGGCGCAGCGGTCG
GGCTGAACGGGGGGTTCGTGCACACAGCCCAGCTTGGAGCGAACGACCTACACC
GAACTGAGATACCTACAGCGTGAGCATTGAGAAAGCGCCACGCTTCCCGAAGGG
AGAAAGGCGGACAGGTATCCGGTAAGCGGCAGGGTCGGAACAGGAGAGCGCAC
GAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTAT
GGAAAAACGCCAGCAACGCGGCCTTTTTACGGTTCCTGGCCTTTTGCTGGCCTTT
TGCTCACATGTTCTTTCCTGCGTTATCCCCTGATTCTGTGGATAACCGTATTACCG
CCTTTGAGTGAGCTGATACCGCTCGCCGCAGCCGAACGACCGAGCGCAGCGAGT
CAGTGAGCGAGGAAGCGGAAGAGCGCCTGATGCGGTATTTTCTCCTTACGCATCT
GTGCGGTATTTCACACCGCAGACCAGCCGCGTAACCTGGCAAAATCGGTTACGG
TTGAGTAATAAATGGATGCCCTGCGTAAGCGGGTGTGGGCGGACAATAAAGTCT
TAAACTGAACAAAATAGATCTAAACTATGACAATAAAGTCTTAAACTAGACAGA
ATAGTTGTAAACTGAAATCAGTCCAGTTATGCTGTGAAAAAGCATACTGGACTTT
TGTTATGGCTAAAGCAAACTCTTCATTTTCTGAAGTGCAAATTGCCCGTCGTATT
AAAGAGGGGCGTGGCCAAGGGCATGGTAAAGACTATATTCGCGGCGTTGTGACA
ATTTACCGAACAACTCCGCGGCCGGGAAGCCGATCTCGGCTTGAACGAATTGTTA
GGTGGCGGTACTTGGGTCGATATCAAAGTGCATCACTTCTTCCCGTATGCCCAAC
TTTGTATAGAGAGCCACTGCGGGATCGTCACCGTAATCTGCTTGCACGTAGATCA
CATAAGCACCAAGCGCGTTGGCCTCATGCTTGAGGAGATTGATGAGCGCGGTGG
CAATGCCCTGCCTCCGGTGCTCGCCGGAGACTGCGAGATCATAGATATAGATCTC
ACTACGCGGCTGCTCAAACCTGGGCAGAACGTAAGCCGCGAGAGCGCCAACAAC
CGCTTCTTGGTCGAAGGCAGCAAGCGCGATGAATGTCTTACTACGGAGCAAGTTC
CCGAGGTAATCGGAGTCCGGCTGATGTTGGGAGTAGGTGGCTACGTCTCCGAAC
TCACGACCGAAAAGATCAAGAGCAGCCCGCATGGATTTGACTTGGTCAGGGCCG
AGCCTACATGTGCGAATGATGCCCATACTTGAGCCACCTAACTTTGTTTTAGGGC
GACTGCCCTGCTGCGTAACATCGTTGCTGCTGCGTAACATCGTTGCTGCTCCATA
ACATCAAACATCGACCCACGGCGTAACGCGCTTGCTGCTTGGATGCCCGAGGCA
TAGACTGTACAAAAAAACAGTCATAACAAGCCATGAAAACCGCCACTGCGCCGT
TACCACCGCTGCGTTCGGTCAAGGTTCTGGACCAGTTGCGTGAGCGCATACGCTA
CTTGCATTACAGTTTACGAACCGAACAGGCTTATGTCAACTGGGTTCGTGCCTTC
ATCCGTTTCCACGGTGTGCGTCACCCGGCAACCTTGGGCAGCAGCGAAGTCGAG
GCATTTCTGTCCTGGCTGGCGAACGAGCGCAAGGTTTCGGTCTCCACGCATCGTC
AGGCATTGGCGGCCTTGCTGTTCTTCTACGGCAAGGTGCTGTGCACGGATCTGCC
CTGGCTTCAGGAGATCGGTAGACCTCGGCCGTCGCGGCGCTTGCCGGTGGTGCTG
ACCCCGGATGAAGTGGTTCGCATCCTCGGTTTTCTGGAAGGCGAGCATCGTTTGT
TCGCCCAGGACTCTAGCTATAGTTCTAGTGGTTGGCCTACGTACCCGTAGTGGCT
ATGGCAGGGCTTGCCGCCCCGACGTTGGCTGCGAGCCCTGGGCCTTCACCCGAAC
TTGGGGGTTGGGGTGGGGAAAAGGAAGAAACGCGGGCGTATTGGTCCCAATGGG
GTCTCGGTGGGGTATCGACAGAGTGCCAGCCCTGGGACCGAACCCCGCGTTTAT
GAACAAACGACCCAACACCCGTGCGTTTTATTCTGTCTTTTTATTGCCGTCATAGC
GCGGGTTCCTTCCGGTATTGTCTCCTTCCGTGTTTCAGTTAGCCTCCCCCATCTCC
CGGTACCGCATGCTATGCATCAGCTGCTAGCACCATGGCTCGAGATCCCGGGTGA
TCAAGTCTTCGTCGAGTGATTGTAAATAAAATGTAATTTACAGTATAGTATTTTA
ATTAATATACAAATGATTTGATAATAATTCTTATTTAACTATAATATATTGTGTTG
GGTTGAATTAAAGGTCCGTATACTCCGGAATATTAATAGATCATGGAGATAATTA
AAATGATAACCATCTCGCAAATAAATAAGTATTTTACTGTTTTCGTAACAGTTTT
GTAATAAAAAAACCTATAAATATTCCGGATTATTCATACCGTCCCACCATCGGGC
GCGGATCCCGGTCCGAAGCGCGCGGAATTCAAAGGCCTACGTCGACGAGCTCAC
TAGTCGCGGCCGCTTTCGAATCTAGAGCCTGCAGTCTCGACAAGCTTGTCGAGAA
GTACTAGAGGATCATAATCAGCCATACCACATTTGTAGAGGTTTTACTTGCTTTA
AAAAACCTCCCACACCTCCCCCTGAACCTGAAACATAAAATGAATGCAATTGTTG
TTGTTAACTTGTTTATTGCAGCTTATAATGGTTACAAATAAAGCAATAGCATCAC
AAATTTCACAAATAAAGCATTTTTTTCACTGCATTCTAGTTGTGGTTTGTCCAAAC
TCATCAATGTATCTTATCATGTCTGGATCTGATCACTGCTTGAGCCTAGGAGATC
CGAACCAGATAAGTGAAATCTAGTTCCAAACTATTTTGTCATTTTTAATTTTCGTA
TTAGCTTACGACGCTACACCCAGTTCCCATCTATTTTGTCACTCTTCCCTAAATAA
TCCTTAAAAACTCCATTTCCACCCCTCCCAGTTCCCAACTATTTTGTCCGCCCACA
GCGGGGCATTTTTCTTCCTGTTATGTTTTTAATCAAACATCCTGCCAACTCCATGT
GACAAACCGTCATCTTCGGCTACTTT
>GFP sequence-downstream of Pph promoter (SEQ ID NO: 2)
(SEQ ID NO: 2)
GTTAAACGTTTTGTTTTCTAGCAATGGCAAACAAGCTTTTCCTCGTCTGCGCAACT
TTCGCCCTCTGCTTCCTCCTCACCAACGCCGTGAGCAAGGGCGAGGAGCTGTTCA
CCGGGGTGGTGCCCATCCTGGTCGAGCTGGACGGCGACGTAAACGGCCACAAGT
TCAGCGTGTCCGGCGAGGGCGAGGGCGATGCCACCTACGGCAAGCTGACCCTGA
AGTTCATCTGCACCACCGGCAAGCTGCCCGTGCCCTGGCCCACCCTCGTGACCAC
CCTGACCTACGGCGTGCAGTGCTTCAGCCGCTACCCCGACCACATGAAGCAGCA
CGACTTCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGGCGAC
ACCCTGGTGAACCGCATCGAGCTGAAGGGCATCGACTTCAAGGAGGACGGCAAC
ATCCTGGGGCACAAGCTGGAGTACAACTACAACAGCCACAACGTCTATATCATG
GCCGACAAGCAGAAGAACGGCATCAAGGTGAACTTCAAGATCCGCCACAACATC
GAGGACGGCAGCGTGCAGCTCGCCGACCACTACCAGCAGAACACCCCCATCGGC
GACGGCCCCGTGCTGCTGCCCGACAACCACTACCTGAGCACCCAGTCCGCCCTGA
GCAAAGACCCCAACGAGAAGCGCGATCACATGGTCCTGCTGGAGTTCGTGACCG
CCGCCGGGATCACTCTCGGCATGGACGAGCTGTACAAGCACCACCATCACCATC
ACTCCGCGGACGAAGTTGATTCTGAGGCATTCGCCTGCAAGCCCTGCTTCAGCAC
CCACCTCTCAGATATTGAGACCAACACGACCGCCGCTGCCGGTTTCATGGTCCTT
CAGGACATCAACTGCTTCCGACCTCACGGAGTCTCAGCAGCTCAAGAGAAGATT
TCCTTCGGAAAGAGCTCCCAGTGCCGTGAAGCCGTCGGTACTCCACAGTACATCA
CCATCACCGCTAACGTTACCGACGAATCATACTTGTACAACGCTGACCTCTTGAT
GCTTTCCGCTTGCCTTTTCTACGCCTCAGAAATGAGCGAGAAGGGCTTCAAAGTC
ATCTTCGGAAACGTCTCTGGCGTTGTTTCCGCTTGCGTCAACTTCACAGATTACGT
GGCCCACGTTACCCAACACACCCAGCAGCACCATCTCGTTATTGATCACATCAGA
TTGCTCCACTTCCTTACACCAAGCAAAGACGAGCTTTGAACTCTCTCTTTCTCTCT
CTCCAAGTCTCCTTCACTCTTTCG
>Mr-RNA-2-downstream of P10 promoter (SEQ ID NO: 3)
(SEQ ID NO: 3)
AAAGGATATTCGATATTCTATCATCTTTAACATCAAGATGGCTAGAGGTAAACAA
AATTCTAATCAGATTCAAAACAATAGTAACGCAAACGGCAAGCGCCGTAAGCGT
AATCGAAGGAATCGCAATCCGCAGACGGTTCCCAACTTTAACCCCATTGTCGCAA
AGCCGACGGTTGCCCCACTTCAAACTAACATTAGAAGTGCTAGGAGTGACGTTA
ATGCCATCACTGTTTTAAATGGCAGTGATTTTCTTACAACTGTCAAAGTCCGAGG
TTCTAACAATTTAACTGATTCCAAGTCTAGAATCTTGGTTAAGCAACCAATTTCT
GCGAGTTCTTTTCTTGGTACCAGAATTTCTGGTCTATCGCAATTTTGGGAGCGTTA
TAGATGGCACAAGGCTGCAGTTAGATATGTTCCTGCAGTACCCAATACTTTAGCT
TGCCAACTTATTGGTTACATCGATACAGATCCACTAGATGACCCCAACGTTATCC
TCGATGTCGATCAGTTACTTAGGCAGGCTACGTCACAAGTGGGTGCGCGGCAGT
GGAATTTCTCTGATACAACAACTATTCCATTGATTGTCAGGCGTGATGATCAATT
GTACTATACTGGTCAAGATAAAGAGAACGTTCGTTTCTCTCAACAGGGTGTATTT
TACCTCTTGCAAGTGACTACACTACTTAATATTAGTGGTGAAGCCATTACAAATG
ATTTAATTTCAGGTTCACTATATTTAGATTGGGTCTGTGGATTTTCCATGCCACAA
ATTAATCCTACACCAGTGGAAGTTTCACAGCTAACTTACAATGCGGATACTATTG
GCAATTGGGTTCCACCAACAGAACTCAAGCAAACTTATACTCAAGATATTACTGG
TTTGAAGCCAAATTCTAAATTTATTATCGTACCCTATATGGATAGAGTAAGTTCT
GAAGTACTGCAGAAGTGCACAATCACTTGTAATGAGGTTGACGCAGTTGGTTCA
ATCTCATATTTCGATACTAGCGCTATCAAATGTGATGGGTACATATCATTTCAGG
CCAATAGCATTGGTGAAGCAACCTTCACCTTAGTGACCGATTATCAGGGTGCGGT
TGACCCTAAACCCTATCAGTATAGGATTATCAGAGCTATCGTCGGCAATAATTAG
GTTGTCATATCTAGCACATGAT
>Mr-RNA-1-downstream of Pph promoter (SEQ ID NO: 4)
(SEQ ID NO: 4)
GTTAAACGTTTTGTTTTCTAGCAATGAAGTGCAATAATCTTCTCTACGGTGTGGA
ACTCCCCATGTGCTTGCAATCGAGCATGGGCATGGTGGTTGTTACCGGACTCGCC
ATAGTGTATTTGGTAATTTACATCCTCATTTACTCATTTACACATGGACCACGACT
CAGCCGCAGATTAGCTTCCTATGTGGTAATCGGACCTTATGAAGCTATTGCTAAA
AGCCGACCCGCGATGGCCCTACAAAGGGCCATGGTTGATTTGACACGACGGGAC
GTGCGTACTGACTGGTACCCATTGAATAATTTGTTGATGAACAAACCTCACCGCT
TATCTGAAAATGGACACAAAACTTCTGGAGCGGTTCGAGATGCAGCTAGAAATT
TAATTACTAGCGCTATAACATCACTGGGTATGGATAAGTATGAAATATCACCAGG
TGGCCACACTGTCGACGAGCAATTGGCATCTCATCGACATTATGCTGTTAATGAC
TTACATCGAGCATCAGCAGATGACGCAGTTAAAGAAAACGCTGTCATTGTGGCT
ATTGATACGGACTACTATCTACGTGATCCATCAATCTACTTTTCAAATAATAATC
CATTTATTCTGCATACTTTTCAACCAATAACTGTTGCAGGTAAAGATGGAGATGT
GAGGTTCACTATTAGTGACAACCAAGTGGACTATAGGGTAGATGGTGGTGGTAG
GTGGCAACATAAAGTTTGGAATTGGTGTGATTATGGTGAATTCCTATATTTAAAG
AACATCCTAGGTATTCTTAGCATTAATTGGTGGCTAAGTTTCTTTGGTATCAGAA
AGGTTATATACCAAAAGATCCAACACGCACGTCCCTGGGTGGATTGTCCGAACC
GGGCCTTAGTTTGGGGATTACCTCAATTCACTAGGTATATGATWACTTGGTTGCC
AATTGAGATGAACGCACGTGAATTGAGTAGAGTCAACTATCAGTGTGCTAGCAG
ACCTGGCTGGAATTGTTTAGTTGACCACACTAGCACCAAACTTGTAGCCAGCATA
GGTCGAGAGGGTAATGACTGTCATGTTGAACTTGCTAAGGAGGACTTAGATGTT
GTTCTAGGCTTAAGTACCATGCAGTCAGTTACTTCAAGACTTTTACAAATGGGTT
ACAAGCAACCACAAACTTTGGCTACGGTTTGTCAGTTCTACAACAGTGCCGCTTA
TGATATACTAAGCTGTAGCATAGTTGCTAGACCTTCACAACCACCTGTCCACTGG
CCATTGGCGTCTGAAATTGATCAGCCTACAACTTCTTTTCGTAATTACTCCAACA
ATATTGTGACCTGTGGTAATCTTTGCCCCCAACTTAAACGTTGGGAGGTTTTAAG
TAATTCTTTAGAACATCGTGTTACCATGGTGGCCAATAATAAGGTTCCAACACCT
CGCATAGCCAGGTTTGCTGAAGAATACGTCCGCCTGGTAGTTCCCGAAGCGAAT
GTAGGTGTACCTTATAGTTTGGAAGATGCGCGTAAAGAACTTGATAAACCCACG
CAAGTCAATGCGGTAAACCAAATTTGGGAAACAGTCGACATGGAAGTCCGCCGA
TTAATTGAAGCATTTGTGAAGAATGAACCGACCAATAAATCTGGTCGCATAATAT
CATCCTTTGCGGACTCAAGATTCTTGTTGAAATTTTCCACATATACGCTTGCCTTT
CGAGATGAAGTATTACATGCTGAACATAATCGACATTGGTTTTGTCCTGGATTGA
CACCTAATGAGATAGCAGATAAAGTCTGCGACTATGTTCGTGGTGTTGCGACACC
TGCAGAAGGTGATTTTAGCAACTTTGACGGAAGAGTATCTGCTTGGTGTCAAGAG
AACGTGATGAATGCGGTTTACCATAGATGGTTTAACCGTAAGTTTTCCAAGGAAT
TGCAGAAGTATACATCAATGTTGGTTAGTTGCCCAGCTCGAGCTAAGCGTTTTGG
TTTCCAATATGAACCGGGAGTGGGGGTTAAGAGTGGTAGTCCAACCACCTGTGA
CCTTAATTCAGTTCTAAATAACTTCACTCAATACGCAGCAGTTAGGCTGACTAAA
CCAGACCTCTCACCACAAGAAGCCTTTGAACAAACTGGCTTAAGTTTCGGCGACG
ATTCACTATTTGACAAGCAATATCAACTCAGATGGAATTACGTCGTCGAACAACT
TGGTATGGAACTCAAGGTTGAACCCTTTGACCCCAGTAATGGTGTGACTTTTCTT
GCTCGTGTTTTTCCTGATCCTTATAGTACAAACACTAGTTTTCAGGATCCACTAAG
AACGTGGAGAAAGTTGAACATGACTTCACGCACGTGTGTACCTGTCGAGTCAGC
AGCTCTTGATCGTGTCAGTGGATATTTAGTAACTGACAAGTACTCTCCAGTAACA
AGTGAGTACTGTCACATGATTGAGAGGTGTTATATGAACACTGCCGAAAGTGTA
ACTCGAAGGAGACAACGCAAGGATTGTGACCGTGAGAAACCATACTGGTTAGTG
AGTGGTGGCGCCTGGCCCCAGAGGGAGGGGGACTATGAATTGATGATGAGGGTT
ACCGCAGCCCGTACTGGATTTGAGGAGTCAAAACTTATTAATTTGATTAGCCAGT
TCCAATCAGTTCATGATCCTTGGGACATCAAACCGCTAGACTATCAGGAACCATC
ACCTTATAAAGACACACTAGACATAGATGGCCAGCCGGTAGATGCAGTGGACGA
ACGTCAATATCAAAATGAGCGCAACACAGTCAACTTACGAGCTGGTGCAGCAAT
TTCCCAGATGCCTGTCCCAAGTGTGCCAGGCGGTGAAGACTGCAATCGACAGTCT
GCCGACATGCCAGGACCCAAAAGTAGCGAAGGATCTGAGCAGCTACAAGGCTTG
CCTGAGCAAGATGGAAGCAACCGCCTTCAATGCCACCGACAACCTACTTTCAAA
GCCAAGGGTGGTAGCAACACTAAAGGGAGAAGCCGTAAATCCCGGAACGGAGG
ACGTTCTATCAGCAGCAAAACAGCAAATCCAACAACTCACGAGGTTGGTGGAGG
CAATGGAAAGACCAGAGTTACCATTGCTAAGCGAAGCGGACCTAAGCGACCTCA
TAACGTGGTAAGCAATGCCTGGGAAACTCTGAATGACAACCGATAAACTCTCTCT
TTCTCTCTCTCCAAGTCTCCTTCACTCTTTCG

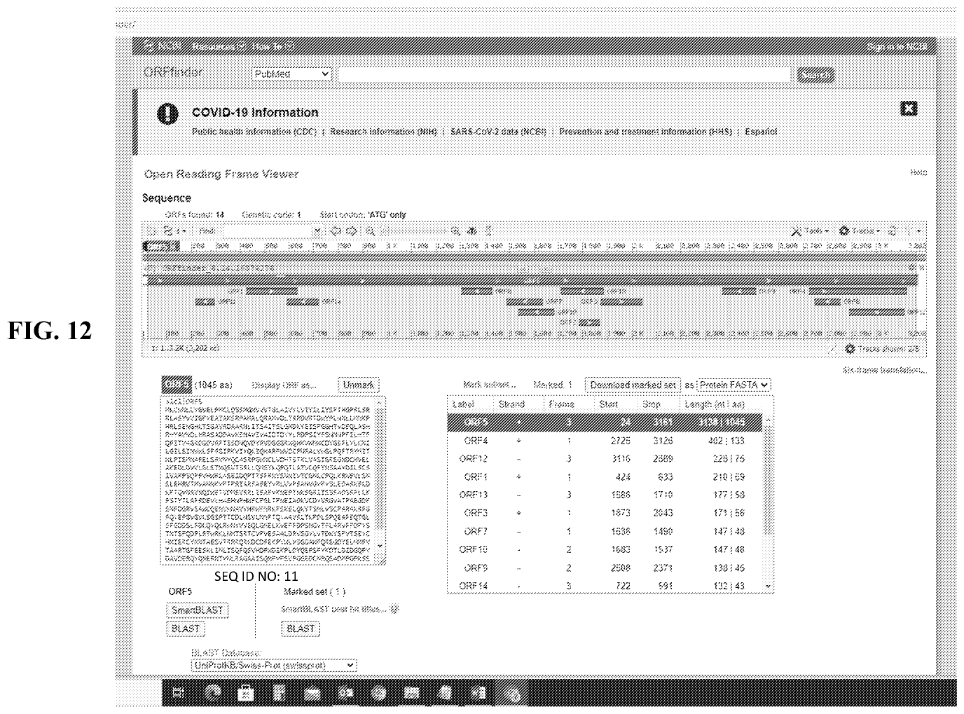

Single underline in SEQ ID NO: 4-5′ and 3′ Non-coding regions
Double underline in SEQ ID NO: 4—RdRp Open Reading Frame, corresponds to nucleotides 24 to 3161
See also FIG. 12.

>Mr-RNA-2-downstream of P10 promoter (SEQ ID NO: 5)
(SEQ ID NO: 5)
AAAGGATATTCGATATTCTATCATCTTTAACATCAAGATGGCTAGAGGTAAACAA
AATTCTAATCAGATTCAAAACAATAGTAACGCAAACGGCAAGCGCCGTAAGCGT
AATCGAAGGAATCGCAATCCGCAGACGGTTCCCAACTTTAACCCCATTGTCGCAA
AGCCGACGGTTGCCCCACTTCAAACTAACATTAGAAGTGCTAGGAGTGACGTTA
ATGCCATCACTGTTTTAAATGGCAGTGATTTTCTTACAACTGTCAAAGTCCGAGG
TTCTAACAATTTAACTGATTCCAAGTCTAGAATCTTGGTTAAGCAACCAATTTCT
GCGAGTTCTTTTCTTGGTACCAGAATTTCTGGTCTATCGCAATTTTGGGAGCGTTA
TAGATGGCACAAGGCTGCAGTTAGATATGTTCCTGCAGTACCCAATACTTTAGCT
TGCCAACTTATTGGTTACATCGATACAGATCCACTAGATGACCCCAACGTTATCC
TCGATGTCGATCAGTTACTTAGGCAGGCTACGTCACAAGTGGGTGCGCGGCAGT
GGAATTTCTCTGATACAACAACTATTCCATTGATTGTCAGGCGTGATGATCAATT
GTACTATACTGGTCAAGATAAAGAGAACGTTCGTTTCTCTCAACAGGGTGTATTT
TACCTCTTGCAAGTGACTACACTACTTAATATTAGTGGTGAAGCCATTACAAATG
ATTTAATTTCAGGTTCACTATATTTAGATTGGGTCTGTGGATTTTCCATGCCACAA
ATTAATCCTACACCAGTGGAAGTTTCACAGCTAACTTACAATGCGGATACTATTG
GCAATTGGGTTCCACCAACAGAACTCAAGCAAACTTATACTCAAGATATTACTGG
TTTGAAGCCAAATTCTAAATTTATTATCGTACCCTATATGGATAGAGTAAGTTCT
GAAGTACTGCAGAAGTGCACAATCACTTGTAATGAGGTTGACGCAGTTGGTTCA
ATCTCATATTTCGATACTAGCGCTATCAAATGTGATGGGTACATATCATTTCAGG
CCAATAGCATTGGTGAAGCAACCTTCACCTTAGTGACCGATTATCAGGGTGCGGT
TGACCCTAAACCCTATCAGTATAGGATTATCAGAGCTATCGTCGGCAATAATTAG
GTTGTCATATCTAGCACATGAT

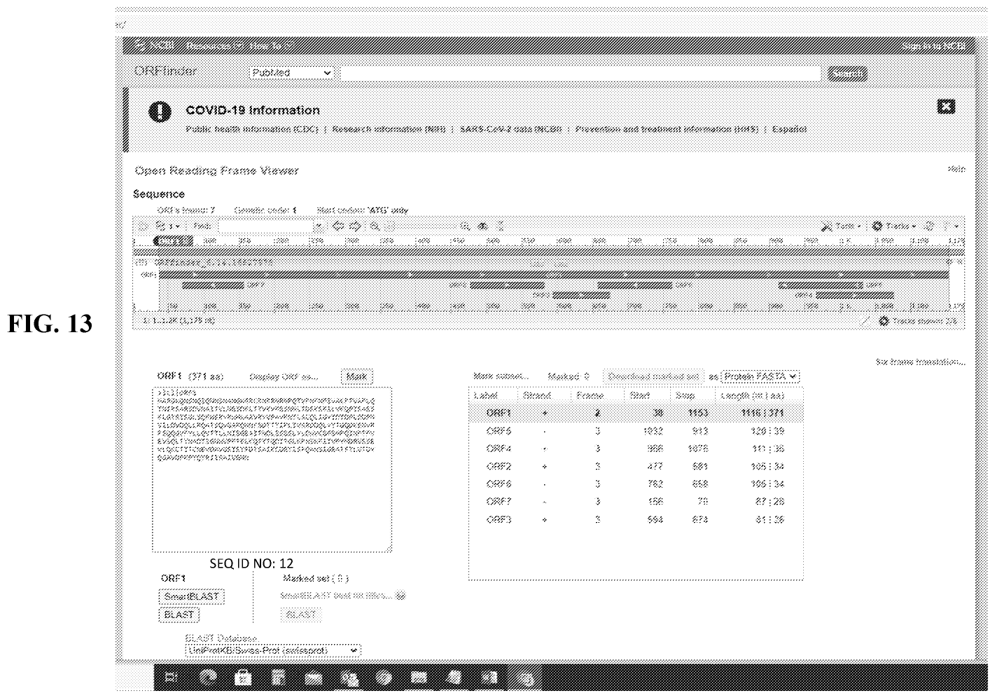

Single underline in SEQ ID NO: 5-5′ and 3′ Non-coding regions
Double underline in SEQ ID NO: 5—Capsid protein open reading from—corresponds to nucleotides 38 to 1153
See also FIG. 13.

>MrNV RNA 1 containing GFP sequence-downstream of Pph promoter (SEQ
ID NO: 6)
(SEQ ID NO: 6)
5′GTTAAACGTTTTGTTTTCTAGCAATGGCAAACAAGCTTTTCCTCGTCTGCGCAA
CTTTCGCCCTCTGCTTCCTCCTCACCAACGCCGTGAGCAAGGGCGAGGAGCTGTT
CACCGGGGTGGTGCCCATCCTGGTCGAGCTGGACGGCGACGTAAACGGCCACAA
GTTCAGCGTGTCCGGCGAGGGCGAGGGCGATGCCACCTACGGCAAGCTGACCCT
GAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAG
CACGACTTCTTCAAGTCCGCCATGCCCGAAGGCTACGTCCAGGAGCGCACCATCT
TCTTCAAGGACGACGGCAACTACAAGACCCGCGCCGAGGTGAAGTTCGAGGGCG
ACACCCTGGTGAACCGCATCGAGCTGAAGGGCATCGACTTCAAGGAGGACGGCA
ACATCCTGGGGCACAAGCTGGAGTACAACTACAACAGCCACAACGTCTATATCA
TGGCCGACAAGCAGAAGAACGGCATCAAGGTGAACTTCAAGATCCGCCACAACA
TCGAGGACGGCAGCGTGCAGCTCGCCGACCACTACCAGCAGAACACCCCCATCG
GCGACGGCCCCGTGCTGCTGCCCGACAACCACTACCTGAGCACCCAGTCCGCCCT
GAGCAAAGACCCCAACGAGAAGCGCGATCACATGGTCCTGCTGGAGTTCGTGAC
CGCCGCCGGGATCACTCTCGGCATGGACGAGCTGTACAAGCACCACCATCACCA
TCACTCCGCGGACGAAGTTGATTCTGAGGCATTCGCCTGCAAGCCCTGCTTCAGC
ACCCACCTCTCAGATATTGAGACCAACACGACCGCCGCTGCCGGTTTCATGGTCC
TTCAGGACATCAACTGCTTCCGACCTCACGGAGTCTCAGCAGCTCAAGAGAAGA
TTTCCTTCGGAAAGAGCTCCCAGTGCCGTGAAGCCGTCGGTACTCCACAGTACAT
CACCATCACCGCTAACGTTACCGACGAATCATACTTGTACAACGCTGACCTCTTG
ATGCTTTCCGCTTGCCTTTTCTACGCCTCAGAAATGAGCGAGAAGGGCTTCAAAG
TCATCTTCGGAAACGTCTCTGGCGTTGTTTCCGCTTGCGTCAACTTCACAGATTAC
GTGGCCCACGTTACCCAACACACCCAGCAGCACCATCTCGTTATTGATCACATCA
GATTGCTCCACTTCCTTACACCAAGCAAAGACGAGCTTTGAACTCTCTCTTTCTCT
CTCTCCAAGTCTCCTTCACTCTTTCG3′

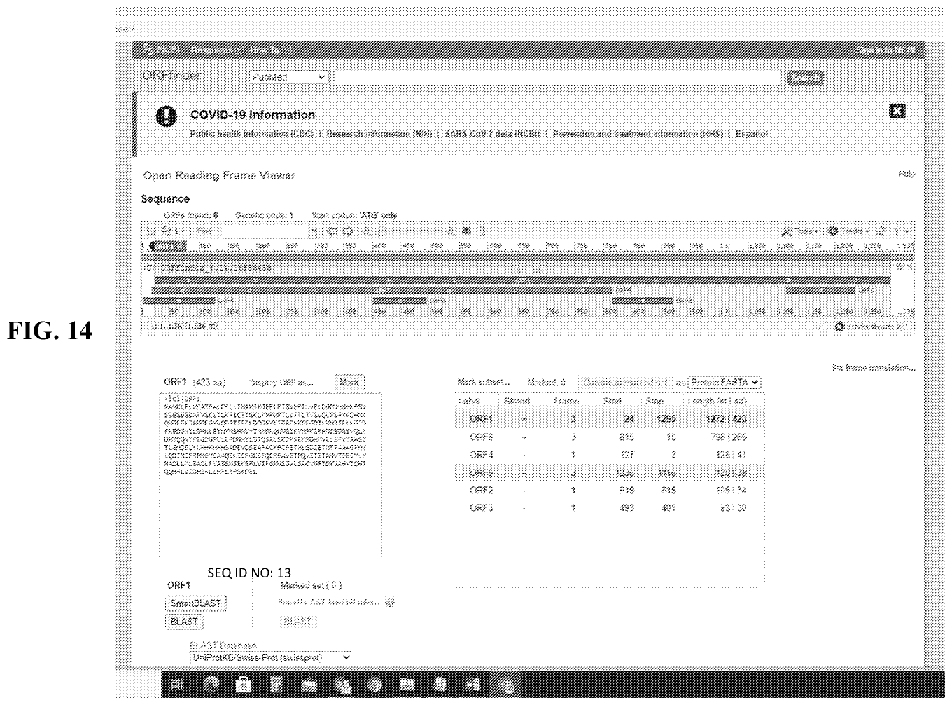

Single underline in SEQ ID NO: 6-5′ and 3′ Non-coding regions
Double underline—GFP Open reading Frame: corresponds to nucleotides 24 to 1295
See also FIG. 14.

### Example 3—lhRNA Delivery for Treatment of Pathogens

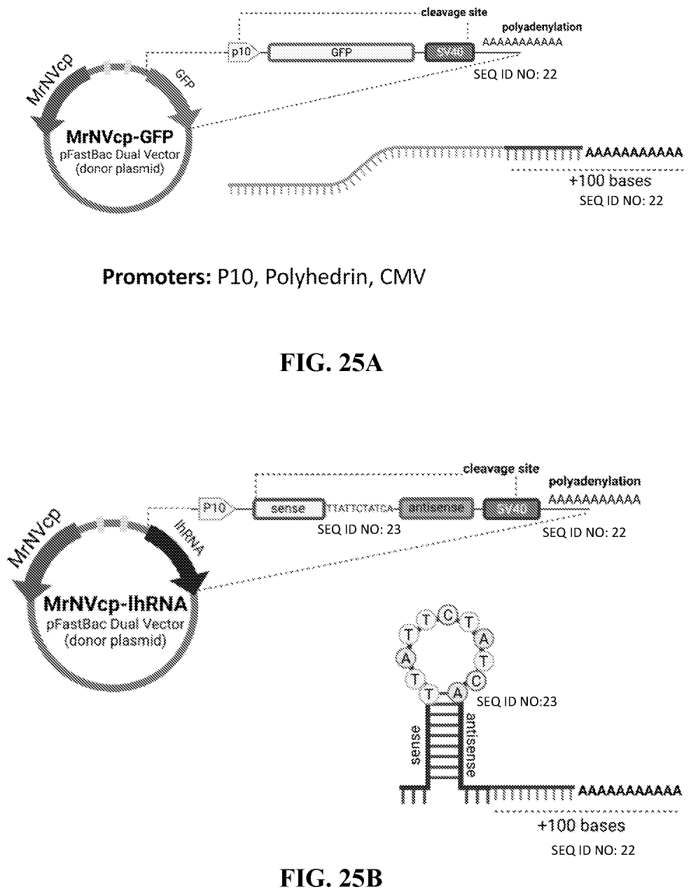

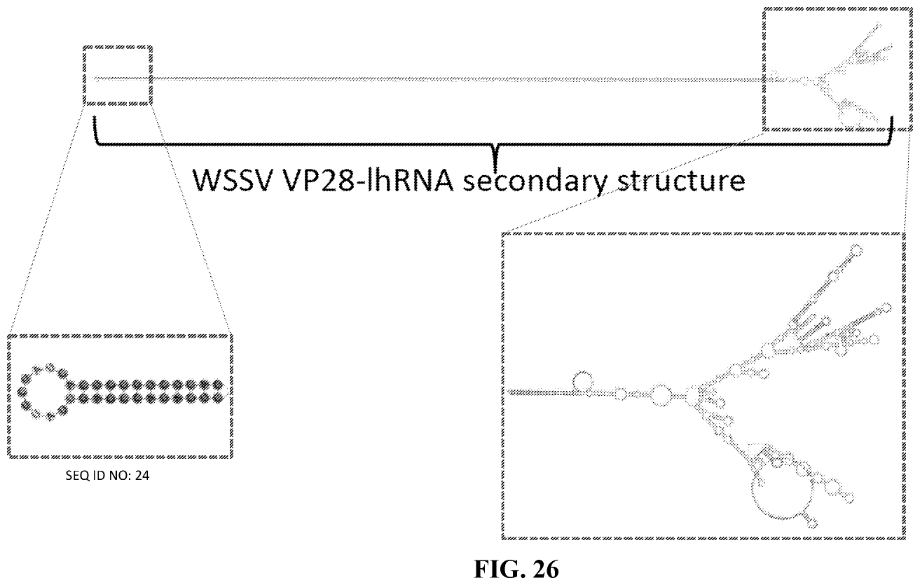

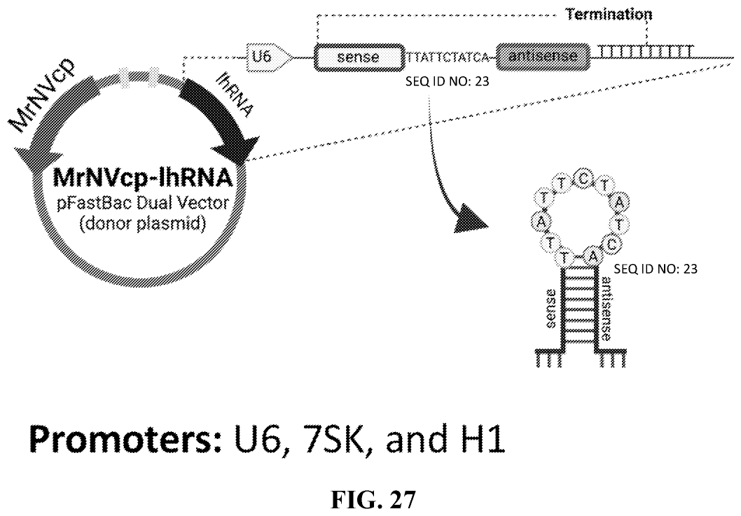

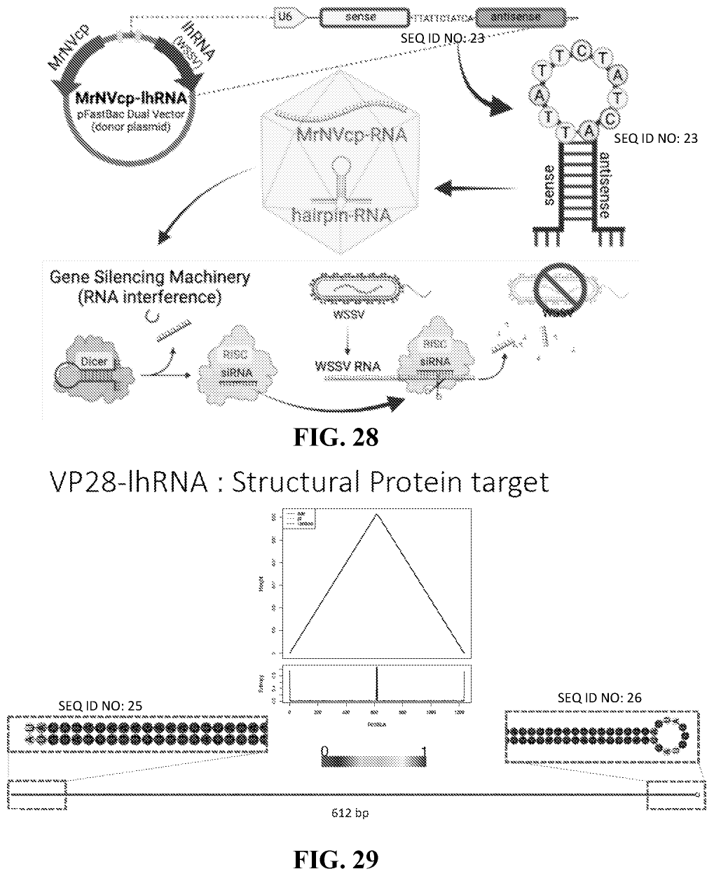

Long hairpin RNA constructs within the nodaviral vector of the present disclosure, (see also e.g., Examples 1-2) were generated. As described herein a therapeutic nucleic acid can be inserted within the nodaviral vector of the present disclosure such that its expression is driven by a Pol II or Pol III promoter. FIG. 25A-28B shows an exemplary strategy for Pol II transcription of a reporter gene (FIG. 25A) or a RNAi molecule (FIG. 25B) in in an embodiment of the nodaviral vector of the present description. Due to different transcription termination sequence recognition by Pol II and Pol III polymerases (see e.g., Gao et al., Mol. Ther. 2018. 10:36-44), it may be advantageous for some therapeutic nucleic acids to be under the control of one type of promoter as opposed to the other. For example, some therapeutic nucleic acids may develop undesirable secondary structures when their expression is driven by a Pol II promoter. For example, some hRNAs, not limited to long hairpin RNAs (lhRNA), can produce undesirable secondary structures (see e.g., FIG. 26). As such, an hRNA construct (e.g., an lhRNA construct) was generated where the lhRNA expression is driven by a Pol III promoter, such as a U6, 7SK, or H1 promoter (see e.g., FIG. 27-29).

Exemplary lhRNA targeting GFP for proof of concept (SEQ ID NO: 7) and lhRNAs targeting WSSV RNA (SEQ ID NOs: 8-10) were developed as shown below. These and others developed in accordance with the description here can be experimentally validated e.g., using an approach described in e.g., the Working Examples herein.

SEQ ID NO: 7—GFP_targeting_long_hairpin_RNA. Single underlined region is the sense sequence of the lhRNA. The hairpin loop sequence is shown in bold, underlined, and italics. The antisense sequence of the lhRNA is shown with double underlining.

(SEQ ID NO: 7)
CCTCTCAGATATTGAGACCAACACGACCGCCGCTGCCGGTTTCATGGTCCTTCAG
GACATCAACTGCTTCCGACCTCACGGAGTCTCAGCAGCTCAAGAGAAGATTTCCT
TCGGAAAGAGCTCCCAGTGCCGTGAAGCCGTCGGTACTCCACAGTACATCACCA
TCACCGCTAACGTTACCGACGAATCATACTTGTACAACGCTGACCTCTTGATGCT
TTCCGCTTGCCTTTTCTACGCCTCAGAAATGAGCGAGAAGGGCTTCAAAGTCATC
TTCGGAAACGTCTCTGGCGTTGTTTCCGCTTGCGTCAACTTCACAGATTACGTGG
CCCACGTTACCCAACACACCCAGCAGCACCATCTCGTTATTGATCACATCAGATT
GCTCCACTTCCTTACACCAAGCAAAGACGAGCTTTGAACTCTCTCTTTCTCTCTCT
CCAAGTCTCCTTCACTCTTTCGTTTTATTCTATCATTCGAAAGAGTGAAGGAGACT
TGGAGAGAGAGAAAGAGAGAGTTCAAAGCTCGTCTTTGCTTGGTGTAAGGAAGT
GGAGCAATCTGATGTGATCAATAACGAGATGGTGCTGCTGGGTGTGTTGGGTAA
CGTGGGCCACGTAATCTGTGAAGTTGACGCAAGCGGAAACAACGCCAGAGACGT
TTCCGAAGATGACTTTGAAGCCCTTCTCGCTCATTTCTGAGGCGTAGAAAAGGCA
AGCGGAAAGCATCAAGAGGTCAGCGTTGTACAAGTATGATTCGTCGGTAACGTT
AGCGGTGATGGTGATGTACTGTGGAGTACCGACGGCTTCACGGCACTGGGAGCT
CTTTCCGAAGGAAATCTTCTCTTGAGCTGCTGAGACTCCGTGAGGTCGGAAGCAG
TTGATGTCCTGAAGGACCATGAAACCGGCAGCGGCGGTCGTGTTGGTCTCAATAT
CTGAGAGG

SEQ ID NO: 8—WSSV_targeting_VP28_long_hairpin_RNA. Single underlined region is the sense sequence of the lhRNA. The hairpin loop sequence is shown in bold, underlined, and italics. The antisense sequence of the lhRNA is shown with double underlining.

(SEQ ID NO: 8)
ATGGATCTTTCTTTCACTCTTTCGGTCGTGTCGGCCATCCTCGCCATCACTGCTGT
GATTGCTGTATTTATTGTGATTTTTAGGTATCACAACACTGTGACCAAGACCATC
GAAACCCACACAGGCAATATCGAGACAAACATGGATGAAAACCTCCGCATTCCT
GTGACTGCTGAGGTTGGATCAGGCTACTTCAAGATGACTGATGTGTCCTTTGACA
GCGACACCTTGGGCAAAATCAAGATCCGCAATGGAAAGTCTGATGCACAGATGA
AGGAAGAAGATGCGGATCTTGTCATCACTCCCGTGGAGGGCCGAGCACTCGAAG
TGACTGTGGGGCAGAATCTCACCTTTGAGGGAACATTCAAGGTGTGGAACAACA
CATCAAGAAAGATCAACATCACTGGTATGCAGATGGTGCCAAAGATTAACCCAT
CAAAGGCCTTTGTCGGTAGCTCCAACACCTCCTCCTTCACCCCCGTCTCTATTGAT
GAGGATGAAGTTGGCACCTTTGTGTGTGGTACCACCTTTGGCGCACCAATTGCAG
CTACCGCCGGTGGAAATCTTTTCGACATGTACGTGCACGTCACCTACTCTGGCAC
TGAGACCGAGTTTTATTCTATCATTCTCGGTCTCAGTGCCAGAGTAGGTGACGTG
CACGTACATGTCGAAAAGATTTCCACCGGCGGTAGCTGCAATTGGTGCGCCAAA
GGTGGTACCACACACAAAGGTGCCAACTTCATCCTCATCAATAGAGACGGGGGT
GAAGGAGGAGGTGTTGGAGCTACCGACAAAGGCCTTTGATGGGTTAATCTTTGG
CACCATCTGCATACCAGTGATGTTGATCTTTCTTGATGTGTTGTTCCACACCTTGA
ATGTTCCCTCAAAGGTGAGATTCTGCCCCACAGTCACTTCGAGTGCTCGGCCCTC
CACGGGAGTGATGACAAGATCCGCATCTTCTTCCTTCATCTGTGCATCAGACTTT
CCATTGCGGATCTTGATTTTGCCCAAGGTGTCGCTGTCAAAGGACACATCAGTCA
TCTTGAAGTAGCCTGATCCAACCTCAGCAGTCACAGGAATGCGGAGGTTTTCATC
CATGTTTGTCTCGATATTGCCTGTGTGGGTTTCGATGGTCTTGGTCACAGTGTTGT
GATACCTAAAAATCACAATAAATACAGCAATCACAGCAGTGATGGCGAGGATGG
CCGACACGACCGAAAGAGTGAAAGAAAGATCCAT

SEQ ID NO: 9—WSSV_targeting_ICP11_long_hairpin_RNA. Single underlined region is the sense sequence of the lhRNA. The hairpin loop sequence is shown in bold, underlined, and italics. The antisense sequence of the lhRNA is shown with double underlining.

(SEQ ID NO: 9)
GCTGGTGGGGGATGATACTAGTAGATATGAAGAAGTGATGAAGACTTTT
GATACTGTTGAGGCAGTCAGGAAGAGTGATCTAGATGACCGTGTTTACA
TGGTGTGCCTAAAGCAGGGATCTACTTTTGTCCTCAATGGAGGCATCGA
AGAATTGCGTCTTTTGACTGGAGATTCAACGCTGGAGATTCAACCCATG
ATTGTGCCTTTTATTCTATCATTGGCACAATCATGGGTTGAATCTCCAG
CGTTGAATCTCCAGTCAAAAGACGCAATTCTTCGATGCCTCCATTGAGG
ACAAAAGTAGATCCCTGCTTTAGGCACACCATGTAAACACGGTCATCTA
GATCACTCTTCCTGACTGCCTCAACAGTATCAAAAGTCTTCATCACTTC
TTCATATCTACTAGTATCATCCCCCACCAGC

SEQ ID NO: 10—WSSV_targeting_VP19_long_hairpin_RNA. Single underlined region is the sense sequence of the lhRNA. The hairpin loop sequence is shown in bold, underlined, and italics. The antisense sequence of the lhRNA is shown with double underlining.

(SEQ ID NO: 10)
ATGGCCACCACGACTAACACTCTTCCTTTCGGCAGGACCGGAGCCCAGG
CCGCTGGCCCTTCTTACACCATGGAAGATCTTGAAGGCTCCATGTCTAT
GGCTCGCATGGGTCTCTTTTTGATCGTTGCTATCTCAATTGGTATCCTC
GTCCTGGCCGTCATGAATGTATGGATGGGACCAAAGAAGGACAGCGATT
CTGACACTGATAAGGTCACCGATGATGATGACGACACTGCCAACGATAA
CGATGATGAGGACAAATATAAGAACAGGACCAGGGATATGATGCTTCTG
GCTGGGTCCGCTCTTCTGTTCCTCGTTTCCGCCGCCACCGTTTTTATGT
CTTACCCCAAGAGGAGGCAGTTTTATTCTATCATTCTGCCTCCTCTTGG
GGTAAGACATAAAAACGGTGGCGGCGGAAACGAGGAACAGAAGAGCGGA
CCCAGCCAGAAGCATCATATCCCTGGTCCTGTTCTTATATTTGTCCTCA
TCATCGTTATCGTTGGCAGTGTCGTCATCATCATCGGTGACCTTATCAG
TGTCAGAATCGCTGTCCTTCTTTGGTCCCATCCATACATTCATGACGGC
CAGGACGAGGATACCAATTGAGATAGCAACGATCAAAAAGAGACCCATG
CGAGCCATAGACATGGAGCCTTCAAGATCTTCCATGGTGTAAGAAGGGC
CAGCGGCCTGGGCTCCGGTCCTGCCGAAAGGAAGAGTGTTAGTCGTGGT
GGCCAT

One of ordinary skill in the art will understand adjustments needed to the procedure to determine efficacy of one or more candidates in view of the description provided herein.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. An engineered cargo delivery system polynucleotide comprising:
an engineered gamma or beta nodavirus polynucleotide comprising
   a nodavirus capsid polypeptide construct comprising a gamma or beta nodavirus RNA2 encoding polynucleotide or a capsid forming domain thereof operatively coupled to one or more regulatory elements; and
   a cargo delivery construct comprising a 5′ untranslated region (UTR) of RNA1 of a native gamma or beta nodavirus, a 3′ UTR of RNA1 of a native gamma or beta nodavirus, or both; and a cargo polynucleotide, wherein the cargo polynucleotide is operatively coupled to the 5′ UTR of RNA1 of a native gamma or beta nodavirus, the 3′ UTR of RNA1 of a native gamma or beta nodavirus, or both, and wherein the cargo polynucleotide is operatively coupled to one or more regulatory elements.

2. The engineered cargo delivery system polynucleotide of aspect 1, wherein the engineered cargo delivery system polynucleotide does not contain a nodavirus RNA dependent polymerase encoding polynucleotide.

3. The engineered cargo delivery system polynucleotide of any one of aspects 1-2, wherein the engineered cargo delivery system polynucleotide does not contain any portion of the translated region of beta or gamma nodavirus RNA1.

4. The engineered cargo delivery system polynucleotide of any one of aspects 1-3, wherein the nodavirus capsid polypeptide construct comprises a 5′ untranslated region (UTR) of a native gamma or beta nodavirus RNA 2, and a 3′ UTR of a native gamma or beta nodavirus RNA 2, or both, wherein the gamma or beta nodavirus RNA2 encoding polynucleotide or a capsid forming domain thereof is operatively coupled the 5′ UTR of a native gamma or beta nodavirus RNA 2, the 3′ UTR of a native gamma or beta nodavirus RNA 2, or both.

5. The engineered cargo delivery system polynucleotide of any one of aspects 1-4, wherein the native gamma or beta nodavirus is selected from Macrobrachium rosenbergii nodavirus (MrNV), Penaeus vannamei nodavirus (PvNV), covert mortality nodavirus (CMNV), Farfantepenaeus duorarum nodavirus (FdNV), tiger puffer nervous necrosis virus (TPNNV), striped jack nervous necrosis virus (SJNNV), barfin flounder nervous necrosis virus (BFNNV), red-spotted grouper nervous necrosis virus (RGNNV), or an Egyptian Nile tilapia nervous necrosis virus (Egyptian NT NNV).

6. The engineered cargo delivery system polynucleotide of any one of aspects 1-5, wherein the native gamma nodavirus is MrNV.

7. The engineered cargo delivery system polynucleotide of any one of aspects 1-6, wherein the engineered cargo delivery system polynucleotide comprises 1-10, 1-20, 1-30, 1-40, 1-50, 1-100, 1-150, 1-200, 1-250, 1-300, 1-350, 1-400, 1-500, 1-750, 1-1000, 1-1500, 1-2000, 1-2500, 1-3000, 1-3500 or more of the translated and/or untranslated region of the native gamma or beta nodavirus RNA2.

8. The engineered cargo delivery system polynucleotide of any one of aspects 1-7, wherein the cargo polynucleotide encodes a protein, an RNA, or both.

9. The engineered cargo delivery system polynucleotide of any one of aspects 1-8, wherein the cargo encodes an RNA, wherein the RNA is not translated.

10. The engineered cargo delivery system polynucleotide of any one of aspects 1-9, wherein the cargo polynucleotide encodes an RNA, wherein the RNA is an interfering RNA, a gene silencing oligonucleotide, or a guide RNA for a CRISPR-Cas system.

11. The engineered cargo delivery system polynucleotide of any one of aspects 1-10, wherein the cargo is a hairpin RNA, optionally a long hairpin RNA.

12. The engineered cargo delivery system polynucleotide of aspect 11, wherein the one or more regulatory elements operatively coupled to the cargo polynucleotide comprises a Pol III promoter and optionally a Poll III termination sequence.

13. The engineered cargo delivery system polynucleotide of any one of aspects 1-12, wherein the cargo polynucleotide encodes an RNA, wherein the RNA is an interfering RNA, a gene silencing oligonucleotide, or a guide RNA for a CRISPR-Cas system, and wherein the gene silencing oligonucleotide, the interfering RNA, and the guide RNA are capable of specifically hybridizing with or targeting a pathogenic DNA or RNA.

14. The engineered cargo delivery polynucleotide of aspect 13, wherein the pathogenic DNA or RNA is a pathogenic DNA or RNA from a shrimp, a prawn, and/or a fish pathogen.

15. The engineered cargo delivery system polynucleotide of any one of aspects 13-14, wherein the shrimp, prawn, or fish pathogen is a virus, a bacteria, a fungus, a worm, or a protozoan.

16. The engineered cargo delivery system polynucleotide of any one of aspects 14-15, wherein the shrimp, prawn, or fishpathogen is white spot syndrome virus (WSSV), Yellow head virus (YHV), Gill associated virus (GAV), Infectious hypodermal and hematopoietic necrosis virus (IHHNV), Taura syndrome virus (TSV), Monodon baculovirus (MBV), Hepatopancreatic parvovirus (HPV), Related Australian lymphoid organ virus (LOV), Mourilyan virus (MOV), Laem Singh virus (LSNV), Baculovirus midgut gland necrosis virus (BMNV), Monodon slow growth syndrome (MSGS), Macrobrachium rosenbergii nodavirus (MrNV), Extra small virus (XSV), Spawner-isolated mortality virus (SMV), Lymphoidal parvo-like virus (LPV), Baculovirus penaei (BP), Shrimp iridovirus (SIV), Shrimp hemocyte iridescent virus (SHIV), Reo-like virus (REO) type II and IV, Lymphoid organ vacuolization virus (LOVV), Rhabdovirus of penaeid shrimp (RPS), Infectious myonecrosis virus (IMNV), a nodavirus, a bacteria of the genus Vibrio, a Rickettsiae, a fungs of the genus Langenidium, a fungus of the genus Sirolopidium, a fungus of the genus Fusarium, a protozoan of the genus Micropsoran, a protozoan of the genus Haplospora, a protozoan of the genus Gregarina, a trematode (e.g., of the family Opecoelidae, Microphalliadae, or Echinostomatidae), a cestode, a nematode, Enterocytozoon hepatopenaei (EHP), Infectious myonecrosis virus, or infection with HPR-deleted or HPRO infectious salmon anemia virus, Epizootic Haematopoietic Necrosis (EHN), Infectious Haematopoietic Necrosis (IHN), Infection with koi herpesvirus, Infection with red sea bream iridovirus, Infectious Spleen & Kidney Necrosis Virus (ISKNV), Infection with salmonid alphavirus, Oncorhynchus masou Virus (OMV), Infectious Pancreatic Necrosis (IPN), Viral Encephalopathy and Retinopathy (VER), Spring Viraemia of Carp (SVC), Channel Catfish Virus (CCV) Viral Haemorrhagic Septicaemia (VHS), Lymphocystis, Bacterial Kidney Disease (BKD), Epizootic Ulcerative Syndrome (EUS), or Nervous Necrosis Virus (NNV), Tilapia Lake Virus (TiLV), or any combination thereof.

17. The engineered cargo delivery system polynucleotide of any one of aspects 1-16, wherein the cargo delivery polynucleotide is a gene of interest or portion thereof.

18. The engineered cargo delivery polynucleotide of any one of aspects 1-17, wherein the cargo delivery polynucleotide encodes a Cas protein or portion thereof.

19. An engineered vector comprising:
an engineered cargo delivery system polynucleotide of any one of aspects 1-18.

20. The engineered vector of aspect 19, wherein the vector comprises one or more components of a baculoviral vector.

21. An engineered cargo delivery vector system comprising:
an engineered cargo delivery system polynucleotide and/or engineered vector of any one of aspects 1-20.

22. The engineered cargo delivery vector system of aspect 21, wherein the cargo delivery construct and the engineered polynucleotide nodavirus capsid polypeptide construct are on the same vector.

23. The engineered cargo delivery vector system of aspect 21, wherein the cargo delivery construct and the engineered polynucleotide nodavirus capsid polypeptide construct are on different vectors.

24. The engineered cargo delivery vector or vector system of any one of aspects 19-23, wherein the engineered cargo delivery vector or vector system is capable of producing engineered nodavirus or nodaviral like particles.

25. The engineered cargo delivery vector or vector system of aspects 19-24, wherein the engineered cargo delivery vector or vector system is capable of packaging the cargo polynucleotide into the engineered nodavirus or nodaviral like particles.

26. The engineered cargo delivery vector or vector system of aspects 19-25, wherein the engineered cargo delivery vector or vector system and/or engineered nodavirus or nodaviral like particles produced therefrom is/are replication incompetent.

27. The engineered cargo delivery vector or vector system of any one of aspects 19-26, wherein the cargo delivery construct and/or the nodavirus capsid polypeptide construct is/are included in a baculoviral vector.

28. An engineered nodavirus or nodavirus like particle produced from and/or containing an engineered cargo delivery system polynucleotide and/or engineered cargo delivery vector or vector system of any one of aspects 1-27.

29. A cell or population thereof comprising:
an engineered cargo delivery system polynucleotide, an engineered cargo delivery vector or vector system, an engineered nodavirus or nodavirus like particle or population thereof, and/or a formulation as in any one of aspects 1-27.

30. The cell or population thereof of aspect 29, wherein the cell or population thereof is a bacterial cell or population thereof or an insect cell or population thereof.

31. The cell of any one of aspects 29-30, wherein the cell or population thereof is non-pathogenic to shrimp, prawns, or other crustaceans, or fish, optionally a fin fish.

32. The cell of any one of aspects 29-31, wherein the cell or population thereof is capable of producing nodavirus or nodavirus like particles comprising the cargo polynucleotide.

33. A method of producing engineered gamma or beta nodavirus or nodavirus like particles carrying a cargo:
expressing an engineered cargo delivery system polynucleotide, an engineered cargo delivery vector or vector system, or any combination thereof as in any one of aspects 1-27 in a cell capable of expressing the engineered cargo delivery system polynucleotide, vector, vector system, or any combination thereof.

34. An engineered nodavirus or nodavirus like particle or population thereof produced by the method of aspect 33.

35. A formulation comprising:
an engineered cargo delivery system polynucleotide, an engineered cargo delivery vector or vector system, a cell or population thereof, an engineered nodavirus or virus like particle or population thereof as in any one of aspects 1-32 or 34, or any combination thereof.

36. The formulation of aspect 35, wherein the formulation is a feed formulation and is optionally adapted for shrimp, prawns, or another crustacean, or fish, optionally a fin fish

37. A method of delivering a cargo to a recipient cell or cell population, the method comprising:
delivering an engineered cargo delivery polynucleotide of any one of aspects 1-18, an engineered cargo delivery vector or vector system as in any one of aspects 19-27, a cell or population thereof as in any one of aspects 29-31, an engineered nodavirus or virus like particle of aspects 28 or 34, a formulation as in any one of aspects 35-36, or any combination thereof to a subject comprising the recipient cell or cell population and/or to the recipient cell of cell population.

38. The method of aspect 37, wherein the subject is a shrimp, prawn, other crustacean, or fish, optionally a fin fish.

39. A method of treating and/or preventing a disease in a crustacean or a fish, optionally a fin fish, comprising:
delivering to the crustacean or fish or a cell thereof an engineered cargo delivery polynucleotide of any one of aspects 1-18, an engineered cargo delivery vector or vector system as in any one of aspects 19-27, a cell or population thereof as in any one of aspects 29-31, an engineered nodavirus or virus like particle of aspects 28 or 34, a formulation as in any one of aspects 35-36, or any combination thereof.

40. The method of treating and/or preventing of aspect 39, wherein the disease is caused by a pathogen, wherein the pathogen is white spot syndrome virus (WSSV), Yellow head virus (YHV), Gill associated virus (GAV), Infectious hypodermal and hematopoietic necrosis virus (IHHNV), Taura syndrome virus (TSV), Monodon baculovirus (MBV), Hepatopancreatic parvovirus (HPV), Related Australian lymphoid organ virus (LOV), Mourilyan virus (MOV), Laem Singh virus (LSNV), Baculovirus midgut gland necrosis virus (BMNV), Monodon slow growth syndrome (MSGS), Macrobrachium rosenbergii nodavirus (MrNV), Extra small virus (XSV), Spawner-isolated mortality virus (SMV), Lymphoidal parvo-like virus (LPV), Baculovirus penaei (BP), Shrimp iridovirus (SIV), Shrimp hemocyte iridescent virus (SHIV), Reo-like virus (REO) type II and IV, Lymphoid organ vacuolization virus (LOVV), Rhabdovirus of penaeid shrimp (RPS), Infectious myonecrosis virus (IMNV), a nodavirus, a bacteria of the genus Vibrio, a Rickettsiae, a fungs of the genus Langenidium, a fungus of the genus Sirolopidium, a fungus of the genus Fusarium, a protozoan of the genus Micropsoran, a protozoan of the genus Haplospora, a protozoan of the genus Gregarina, a trematode (e.g., of the family Opecoelidae, Microphalliadae, or Echinostomatidae), a cestode (e.g., of the genus Prochristianella, Parachristianella, or Renibulbus), a nematode (e.g., Spirocamallanus pereirai, Leptolaimus sp. Ascaropsis sp, and Hysterothylacium reliquens), Enterocytozoon hepatopenaei (EHP), or Infectious myonecrosis virus.

41. A kit comprising:
an engineered cargo delivery polynucleotide of any one of aspects 1-18, an engineered cargo delivery vector or vector system as in any one of aspects 19-27, a cell or population thereof as in any one of aspects 29-31, an engineered nodavirus or virus like particle of aspects 28 or 34, a formulation as in any one of aspects 35-36, or any combination thereof.

What is claimed is:

1. An engineered cargo delivery system polynucleotide comprising:
an engineered gamma or beta nodavirus polynucleotide comprising
a nodavirus capsid polypeptide construct comprising a gamma or beta nodavirus RNA2 encoding polynucleotide or a capsid forming domain thereof operatively coupled to one or more regulatory elements; and
a cargo delivery construct comprising a 5′ untranslated region (UTR) of RNA1 of a native gamma or beta nodavirus, a 3′ UTR of RNA1 of a native gamma or beta nodavirus, or both; and a cargo polynucleotide, wherein the cargo polynucleotide is operatively coupled to the 5′ UTR of RNA1 of a native gamma or beta nodavirus, the 3′ UTR of RNA1 of a native gamma or beta nodavirus, or both, and wherein the cargo polynucleotide is operatively coupled to one or more regulatory elements.

2. The engineered cargo delivery system polynucleotide of claim 1, wherein the engineered cargo delivery system polynucleotide does not contain a nodavirus RNA dependent polymerase encoding polynucleotide.

3. The engineered cargo delivery system polynucleotide of claim 1, wherein the engineered cargo delivery system polynucleotide does not contain any portion of the translated region of beta or gamma nodavirus RNA1.

4. The engineered cargo delivery system polynucleotide of claim 1, wherein the nodavirus capsid polypeptide construct comprises a 5′ untranslated region (UTR) of a native gamma or beta nodavirus RNA 2, and a 3′ UTR of a native gamma or beta nodavirus RNA 2, or both, wherein the gamma or beta nodavirus RNA2 encoding polynucleotide or a capsid forming domain thereof is operatively coupled the 5′ UTR of a native gamma or beta nodavirus RNA 2, the 3′ UTR of a native gamma or beta nodavirus RNA 2, or both.

5. The engineered cargo delivery system polynucleotide of claim 1, wherein the native gamma or beta nodavirus is selected from Macrobrachium rosenbergii nodavirus (MrNV), Penaeus vannamei nodavirus (PvNV), covert mortality nodavirus (CMNV), Farfantepenaeus duorarum nodavirus (FdNV), tiger puffer nervous necrosis virus (TPNNV), striped jack nervous necrosis virus (SJNNV), barfin flounder nervous necrosis virus (BFNNV), red-spotted grouper nervous necrosis virus (RGNNV), or an Egyptian Nile tilapia nervous necrosis virus (Egyptian NT NNV).

6. The engineered cargo delivery system polynucleotide of claim 1, wherein the native gamma nodavirus is MrNV.

7. The engineered cargo delivery system polynucleotide of claim 1, wherein the engineered cargo delivery system polynucleotide comprises 1-10, 1-20, 1-30, 1-40, 1-50, 1-100, 1-150, 1-200, 1-250,1-300, 1-350, 1-400, 1-500, 1-750,1-1000, 1-1500, 1-2000, 1-2500, 1-3000, 1-3500 or more of the translated and/or untranslated region of the native gamma or beta nodavirus RNA2.

8. The engineered cargo delivery system polynucleotide of claim 1, wherein the cargo polynucleotide encodes a protein, an RNA, or both.

9. The engineered cargo delivery system polynucleotide of claim 1, wherein the cargo encodes an RNA, wherein the RNA is not translated.

10. The engineered cargo delivery system polynucleotide of claim 1, wherein the cargo polynucleotide encodes an RNA, wherein the RNA is an interfering RNA, a gene silencing oligonucleotide, a guide RNA for a CRISPR-Cas system or a microRNA.

11. The engineered cargo delivery system polynucleotide of claim 10, wherein the cargo is a hairpin RNA, optionally a long hairpin RNA.

12. The engineered cargo delivery system polynucleotide of claim 11, wherein the one or more regulatory elements operatively coupled to the cargo polynucleotide comprises a Pol III promoter and optionally a Poll III termination sequence.

13. The engineered cargo delivery system polynucleotide of claim 1, wherein the cargo polynucleotide encodes an RNA, wherein the RNA is an interfering RNA, a gene silencing oligonucleotide, or a guide RNA for a CRISPR-Cas system, and wherein the gene silencing oligonucleotide, the interfering RNA, and the guide RNA are capable of specifically hybridizing with or targeting a pathogenic DNA or RNA.

14. The engineered cargo delivery polynucleotide of claim 13, wherein the pathogenic DNA or RNA is a pathogenic DNA or RNA from a shrimp, a prawn, and/or a fish pathogen.

15. The engineered cargo delivery system polynucleotide of claim 14, wherein the shrimp, prawn, or fish pathogen is a virus, a bacteria, a fungus, a worm, or a protozoan.

16. The engineered cargo delivery system polynucleotide of claim 14, wherein the shrimp, prawn, or fish pathogen is white spot syndrome virus (WSSV), Yellow head virus (YHV), Gill associated virus (GAV), Infectious hypodermal and hematopoietic necrosis virus (IHHNV), Taura syndrome virus (TSV), Monodon baculovirus (MBV), Hepatopancreatic parvovirus (HPV), Related Australian lymphoid organ virus (LOV), Mourilyan virus (MOV), Laem Singh virus (LSNV), Baculovirus midgut gland necrosis virus (BMNV), Monodon slow growth syndrome (MSGS), Macrobrachium rosenbergii nodavirus (MrNV), Extra small virus (XSV), Spawner-isolated mortality virus (SMV), Lymphoidal parvo-like virus (LPV), Baculovirus penaei (BP), Shrimp iridovirus (SIV), Shrimp hemocyte iridescent virus (SHIV), Reo-like virus (REO) type II and IV, Lymphoid organ vacuolization virus (LOVV), Rhabdovirus of penaeid shrimp (RPS), Infectious myonecrosis virus (IMNV), a nodavirus, a bacteria of the genus Vibrio, a Rickettsiae, a fungs of the genus Langenidium, a fungus of the genus Sirolopidium, a fungus of the genus Fusarium, a protozoan of the genus Micropsoran, a protozoan of the genus Haplospora, a protozoan of the genus Gregarina, a trematode (e.g., of the family Opecoelidae, Microphalliadae, or Echinostomatidae), a cestode, a nematode, Enterocytozoon hepatopenaei (EHP), Infectious myonecrosis virus, or infection with HPR-deleted or HPRO infectious salmon anemia virus, Epizootic Haematopoietic Necrosis (EHN), Infectious Haematopoietic Necrosis (IHN), Infection with koi herpesvirus, Infection with red sea bream iridovirus, Infectious Spleen & Kidney Necrosis Virus (ISKNV), Infection with salmonid alphavirus, Oncorhynchus masou Virus (OMV), Infectious Pancreatic Necrosis (IPN), Viral Encephalopathy and Retinopathy (VER), Spring Viraemia of Carp (SVC), Channel Catfish Virus (CCV) Viral Haemorrhagic Septicaemia (VHS), Lymphocystis, Bacterial Kidney Disease (BKD), Epizootic Ulcerative Syndrome (EUS), or Nervous Necrosis Virus (NNV), Tilapia Lake Virus (TiLV), or any combination thereof.

17. The engineered cargo delivery system polynucleotide of claim 1, wherein the cargo delivery polynucleotide is a gene of interest or portion thereof.

18. The engineered cargo delivery polynucleotide of claim 1, wherein the cargo delivery polynucleotide encodes a Cas protein or portion thereof.

19. An engineered vector comprising:
an engineered cargo delivery system polynucleotide of claim 1.

20. The engineered vector of claim 19, wherein the vector comprises one or more components of a baculoviral vector.

21. An engineered cargo delivery vector system comprising:
an engineered cargo delivery system polynucleotide of claim 1 or an engineered vector including the engineered cargo delivery system polynucleotide.

22. The engineered cargo delivery vector system of claim 21, wherein the cargo delivery construct and the engineered polynucleotide nodavirus capsid polypeptide construct are on the same vector.

23. The engineered cargo delivery vector of claim 19 or vector system thereof, wherein the cargo delivery construct and the engineered polynucleotide nodavirus capsid polypeptide construct are on different vectors.

24. The engineered cargo delivery vector of claim 19 or vector system thereof, wherein the engineered cargo delivery vector or vector system thereof is capable of producing engineered nodavirus or nodaviral like particles.

25. The engineered cargo delivery vector of claim 19 or vector system thereof, wherein the engineered cargo delivery vector or vector system thereof is capable of packaging the cargo polynucleotide into the engineered nodavirus or nodaviral like particles.

26. The engineered cargo delivery vector of claim 19 or vector system thereof, wherein the engineered cargo delivery vector or vector system thereof and/or engineered nodavirus or nodaviral like particles produced therefrom is/are replication incompetent.

27. The engineered cargo delivery vector of claim 19 or vector system thereof, wherein the cargo delivery construct and/or the nodavirus capsid polypeptide construct is/are included in a baculoviral vector.

28. An engineered nodavirus or nodavirus like particle produced from and/or containing an engineered cargo delivery system polynucleotide and/or engineered cargo delivery vector of claim 19 or vector system thereof.

29. A cell or population thereof comprising:
an engineered cargo delivery system polynucleotide of claim 1, an engineered cargo delivery vector or vector system comprising the engineered cargo delivery system polynucleotide, an engineered nodavirus or nodavirus like particle or population thereof produced from the engineered cargo delivery system polynucleotide, and/or a formulation thereof.

30. The cell or population thereof of claim 29, wherein the cell or population thereof is a bacterial cell or population thereof or an insect cell or population thereof.

31. The cell of claim 29, wherein the cell or population thereof is non-pathogenic to shrimp, prawns, or other crustaceans, or fish, optionally a fin fish.

32. The cell of claim 29, wherein the cell or population thereof is capable of producing nodavirus or nodavirus like particles comprising the cargo polynucleotide.

33. A formulation comprising:
an engineered cargo delivery system polynucleotide of claim 1, an engineered cargo delivery vector or vector system thereof comprising the engineered cargo delivery system polynucleotide, a cell or population thereof comprising the engineered cargo delivery system polynucleotide, an engineered nodavirus or virus like particle or population produced by or comprising the engineered delivery cargo delivery system polynucleotide, or any combination thereof.

34. The formulation of claim 33, wherein the formulation is a feed formulation and is optionally adapted for shrimp, prawns, or another crustacean, or fish, optionally a fin fish.

35. A method of producing engineered gamma or beta nodavirus or nodavirus like particles carrying a cargo:
expressing an engineered cargo delivery system polynucleotide of claim 1, an engineered cargo delivery vector or vector system comprising the engineered cargo delivery system polynucleotide in a cell capable of expressing the engineered cargo delivery system polynucleotide, vector, vector system, or any combination thereof.

36. An engineered nodavirus or nodavirus like particle or population thereof produced by the method of claim 35.

37. A method of delivering a cargo to a recipient cell or cell population, the method comprising:
delivering an engineered cargo delivery system polynucleotide of claim 1 to the recipient cell or cell population, a vector or vector system comprising the engineered cargo delivery system polynucleotide, and/or an engineered nodavirus or virus like particle produced by or containing the engineered cargo delivery system polynucleotide to a subject comprising the recipient cell or cell population and/or to the recipient cell of cell population.

38. The method of claim 37, wherein the engineered cargo delivery system polynucleotide of claim 1 to the recipient cell or cell population, a vector or vector system comprising the engineered cargo delivery system polynucleotide, and/or an engineered nodavirus or virus like particle produced by or containing the engineered cargo delivery system polynucleotide is contained in a formulation.

39. The method of claim 38, wherein the formulation is a feed formulation, optionally formulated for shrimp, prawn or other crustacean or a fish, optionally a fin fish.

40. The method of claim 37, wherein the subject is a shrimp, prawn, other crustacean, or fish, optionally a fin fish.

41. A method of treating and/or preventing a disease in a crustacean or a fish, optionally a fin fish, comprising:
delivering to the crustacean or fish or a cell thereof an engineered cargo delivery system polynucleotide of claim 1 to the recipient cell or cell population, a vector or vector system comprising the engineered cargo delivery system polynucleotide, and/or an engineered nodavirus or virus like particle produced by or containing the engineered cargo delivery system polynucleotide.

42. The method of claim 41, wherein the engineered cargo delivery system polynucleotide of claim 1 to the recipient cell or cell population, a vector or vector system comprising the engineered cargo delivery system polynucleotide, and/or an engineered nodavirus or virus like particle produced by or containing the engineered cargo delivery system polynucleotide is contained in a formulation.

43. The method of claim 42, wherein the formulation is a feed formulation, optionally formulated for shrimp, prawn or other crustacean or a fish, optionally a fin fish.

44. The method of treating and/or preventing of claim 41, wherein the disease is caused by a pathogen, wherein the pathogen is white spot syndrome virus (WSSV), Yellow head virus (YHV), Gill associated virus (GAV), Infectious hypodermal and hematopoietic necrosis virus (IHHNV), Taura syndrome virus (TSV), Monodon baculovirus (MBV), Hepatopancreatic parvovirus (HPV), Related Australian lymphoid organ virus (LOV), Mourilyan virus (MOV), Laem Singh virus (LSNV), Baculovirus midgut gland necrosis virus (BMNV), Monodon slow growth syndrome (MSGS), Macrobrachium rosenbergii nodavirus (MrNV), Extra small virus (XSV), Spawner-isolated mortality virus (SMV), Lymphoidal parvo-like virus (LPV), Baculovirus penaei (BP), Shrimp iridovirus (SIV), Shrimp hemocyte iridescent virus (SHIV), Reo-like virus (REO) type II and IV, Lymphoid organ vacuolization virus (LOVV), Rhabdovirus of penaeid shrimp (RPS), Infectious myonecrosis virus (IMNV), a nodavirus, a bacteria of the genus Vibrio, a Rickettsiae, a fungs of the genus Langenidium, a fungus of the genus Sirolopidium, a fungus of the genus Fusarium, a protozoan of the genus Micropsoran, a protozoan of the genus Haplospora, a protozoan of the genus Gregarina, a trematode (e.g., of the family Opecoelidae, Microphalliadae, or Echinostomatidae), a cestode, a nematode, Enterocytozoon hepatopenaei (EHP), Infectious myonecrosis virus, or infection with HPR-deleted or HPRO infectious salmon anemia virus, Epizootic Haematopoietic Necrosis (EHN), Infectious Haematopoietic Necrosis (IHN), Infection with koi herpesvirus, Infection with red sea bream iridovirus, Infectious Spleen & Kidney Necrosis Virus (ISKNV), Infection with salmonid alphavirus, Oncorhynchus masou Virus (OMV), Infectious Pancreatic Necrosis (IPN), Viral Encephalopathy and Retinopathy (VER), Spring Viraemia of Carp (SVC), Channel Catfish Virus (CCV) Viral Haemorrhagic Septicaemia (VHS), Lymphocystis, Bacterial Kidney Disease (BKD), Epizootic Ulcerative Syndrome (EUS), or Nervous Necrosis Virus (NNV), Tilapia Lake Virus (TiLV), or any combination thereof.

45. A kit comprising:
an engineered cargo delivery polynucleotide of any one of claims 1-18, an engineered cargo delivery vector or vector system as in any one of claims 19-27, a cell or population thereof as in any one of claims 29-31, an engineered nodavirus or virus like particle of claim 28 or 34, a formulation as in any one of claims 35-36, or any combination thereof.
